SEQUENCE LISTING (171 sequences)

SEQ ID NO: 1 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 2 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 3 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 4 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 5 - DNA, 1000 bp, Arabidopsis thaliana
  misc_feature (1)..(1000): Ceres Promoter PT0613
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 6 - DNA, 351 bp, Arabidopsis thaliana
  misc_feature (1)..(351): Ceres Promoter PT0625
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 7 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 8 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 9 - DNA, 998 bp, Arabidopsis thaliana
  misc_feature (1)..(998): Ceres Promoter PT0660
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 10 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 11 - DNA, 999 bp, Arabidopsis thaliana
  misc_feature (1)..(999): Ceres Promoter PT0672
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 12 - DNA, 1000 bp, Arabidopsis thaliana
  misc_feature (1)..(1000): Ceres Promoter PT0676
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 13 - DNA, 998 bp, Arabidopsis thaliana
  misc_feature (1)..(998): Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 14 - DNA, 1000 bp, Arabidopsis thaliana
  misc_feature (1)..(1000): Ceres Promoter PT0683
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 15 - DNA, 1000 bp, Arabidopsis thaliana
  misc_feature (1)..(1000): Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 16 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 17 - DNA, 1000 bp, Arabidopsis thaliana
  misc_feature (1)..(1000): Ceres Promoter PT0708
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 18 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 19 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 20 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 21 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 22 - DNA, 1000 bp, Arabidopsis thaliana
  misc_feature (1)..(1000): Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 23 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 24 - DNA, 763 bp, Arabidopsis thaliana
  misc_feature (1)..(763): Ceres Promoter PT0837
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 25 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 26 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 27 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 28 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 29 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 30 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 31 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 32 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 33 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 34 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 35 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 36 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 37 - DNA, 1024 bp, Arabidopsis thaliana
  misc_feature (1)..(1024): Ceres Promoter YP0092
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaat

SEQ ID NO: 38 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 39 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 40 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 41 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 42 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 43 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 44 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 45 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 46 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 47 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 48 - DNA, 1000 bp, Arabidopsis thaliana
  misc_feature (1)..(1000): Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 49 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 50 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 51 - DNA, 1004 bp, Arabidopsis thaliana
  misc_feature (1)..(1004): Ceres Promoter YP0128
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 52 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 53 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 54 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 55 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 56 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 57 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 58 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 59 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 60 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 61 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 62 - DNA, 999 bp, Arabidopsis thaliana
  misc_feature (1)..(999): Ceres Promoter YP0275
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 63 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 64 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 65 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 66 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 67 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 68 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 69 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 70 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 71 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 72 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 73 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 74 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 75 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 76 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 77 - DNA, 667 bp, Arabidopsis thaliana
  misc_feature (1)..(857): Ceres Promoter PD1367
acagttttcttttctcatcttacaacaagtttccaggaggatagagacataaacgaagctcggattgtatcgttctttttagcttttattcacatccgaaagtcctgtagtttagattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttctcctctttcacttcatcacttggcttgtgaattcattaattcctgcgaa

SEQ ID NO: 78 - DNA, 1917 bp, Arabidopsis thaliana
  misc_feature (1)..(1917): Ceres CLONE ID no. 101035
acactgttactctctctctctcttcttcttcttcttcttcttctgcatctcatcgacatcatcctttcccatggctgatgcatacgagccttatcatgttcttcaacaaagccggcgagacaaacttcgtattccatctctcgattcccacttccactttcaccctcctcctcctccttcctccggcggcggaggtggcgtctttcctctcgctgattccgatttcctcgcagccggtggctttcactccaacaacaacaacaaccacatatctaaccctagctacagtaatttcatgggatttctcggtggcccttcttcttcttcatccaccgcagtcgccgtcgccggagatcattcctttaacgccggactttcttccggagacgttcttgtcttcaaacccgagcctctatctctatctttgtcctctcaccctagactcgcttacgatctagtcgttcccggtgttgttaactccggattctgtagatctgccggtgaagccaacgccgccgccgtcaccatcgcgtctagaagctctggtcctctcggacctttcacgggctacgcgtcgattcttaaaggatcaaggttcttgaaaccagcacagatgcttcttgatgagttttgtaatgtgggtcgtgggatttacaccgacaaagtcatcgacgacgatgattcttctctgctttttgatccgacggttgagaatctctgcggtgtttctgatggcggcggaggagataatggaaagaaaaaatcaaaactcatctccatgctcgacgaggtttacaagaggtataagcaatactatgagcagctacaagctgtgatgggatcattcgaatgcgttgcaggtctcgggcacgctgctccgtacgctaacttagccttgaaagcgttgtctaagcatttcaagtgtttgaagaatgctataacggaccagcttcaattcagccacaacaacaagatccaacaacaacaacaatgtggtcatccgatgaactctgagaataagactgattctttaagatttggaggaagtgatagttctagaggcttatgttctgctggtcaaagacatggatttcctgatcatcatgctcctgtttggagaccgcaccgtggcctacccgaacgtgctgttactgttctaagggcttggctcttcgatcatttcttgcatccttatccaacagatacagacaaactcatgctggctaagcagacaggtctctccagaaatcaggtatcgaattggttcataaacgcaagagttagggtttggaagccgatggtggaagagattcacatgctggagactcgacaatctcagagatcttcttcttcctcttggagagacgaacgtactaccaccaccgtcttccctgacaacaacaacaacaacccatcttcgtcctcggcacagcaaagacctaacaactcatctccgcctagacgggcacgaaacgacgacgttcatggcacaaacaacaacaacagctatgtaaacagtgggagcggcggcggtagtgcggttggtttctcgtatggaattgggtcgtcgaatgtgccggtgatgaatagcagcacaaacggaggagtgtctttgacgttagggcttcatcatcagattgggttaccggagccttttccgatgacaactgctcagaggtttgggcttgatggtggtagtggcgatggtggtggtgggtatgaagggcaaaatcgtcagtttgggagagattttattggtggtagtaatcatcagtttctacatgattttgtaggttgagattatttgtgtggaaaggaaaaaatatgtttgacgtttgggtatgtataagaagatatgggggaattgaaatgcatatgatgtgtatattagaatgtttcttcttcagaaaaaaaaaa

SEQ ID NO: 79 - protein, 575 aa, Arabidopsis thaliana
  misc_feature (1)..(575): Ceres CLONE ID no. 101035
MADAYEPYHVLQQSRRDKLRIPSLDSHFHFHPPPPPSSGGGGGVFPLADSDFLAAGGFHSNNNNNHISNPSYSNFMGFLGGPSSSSSTAVAVAGDHSFNAGLSSGDVLVFKPEPLSLSLSSHPRLAYDLVVPGVVNSGFCRSAGEANAAAVTIASRSSGPLGPFTGYASILKGSRFLKPAQMLLDEFCNVGRGIYTDKVIDDDDSSLLFDPTVENLCGVSDGGGGDNGKKKSKLISMLDEVYKRYKQYYEQLQAVMGSFECVAGLGHAAPYANLALKALSKHFKCLKNAITDQLQFSHNNKIQQQQQCGHPMNSENKTDSLRFGGSDSSRGLCSAGQRHGFPDHHAPVWRPHRGLPERAVTVLRAWLFDHFLHPYPTDTDKLMLAKQTGLSRNQVSNWFINARVRVWKPMVEEIHMLETRQSQRSSSSSWRDERTSTTVFPDNNNNNPSSSSAQQRPNNSSPPRRARNDDVHGTNNNNSYVNSGSGGGSAVGFSYGIGSSNVPVMNSSTNGGVSLTLGLHHQIGLPEPFPMTTAQRFGLDGGSGDGGGGYEGQNRQFGRDFIGGSNHQFLHDFVG

SEQ ID NO: 80 - protein, 516 aa, Medicago truncatula
  misc_feature (1)..(516): Public GI no. 92878234
MAEEGFENYYHIPQQSRREKLRYLSQNQTSFIESSPTLNPSFSPLPSLYDPSLISPLDAINSNPFLYQMNHVYNHGGSNSNNNEVMLLKSEPLSLSLSSNKNNQNQMSAANFQRYGSVVDVSRNTVPLGPFTGYASVLKGSRFLKPAQQLLDEICDVGVRAEKIIADADASLMETNHVIGGMINGVDDEDTLGGDGRKNKSRLLTVLDEVCRRYRQYYQQIHAVITSFEYVAGLGNAAPYASLAINAMSKHFRFLKNVITDQLQFIGKSNYHISNRKDESPRFHNGDGAPYSQSPGFMEHVQQPVWRPQRGLPERAVSVLRGWLFEHFLHPYPSDTDKLMLAKQTGLSRNQVSNWFINARVRLWKPMVEEIHMLESQQSPKESQRDEHSRNNLSENNIAENPSTSTDKFIDVAYKRTRNELHNMSVPNHSIASNQQVGNVGVSMMNNGATSNGVSLTLGLHQNHGIGLSEPFNMSAAQRFGLAHQPDSYAASGFQLQNRQFGRDFIGGQLLRDYVG

SEQ ID NO: 81 - protein, 611 aa, Hordeum vulgare subsp. vulgare
  misc_feature (1)..(611): Public GI no. 13752409
MSSPAGGYGGAEAHHHGHMLLHSHAHHMAAAAAASGGQLYHVPQHSRREKLRFPPDAAAEDSPPTPLAPHHQHHQAGAWPPPAFYSYASSSSSYSPHSPTVPQGQQLVLNGLTAQQVTAQQFPHIPTHNFSLSLSSASSNPATAPPTPRKQQEPGGAGPCGPFTGYASVLGRSKFLVPAQRLLEEICDVGGAAAHADRSLPDEGLLDADTMDVADDELDAAGPMYGAEQQWKKTRLISMMEEVCKRYRQYYQQVQSAIASFETVAGFSNAAPFTALALRVMAKHFKTIKEMILSQLRNTSKMPVKGSSMSKDITIFGLGGGGGAPVGGFQRGSSVNGFGQPHNIWRPQRGLPERSVTVLRAWLFEHFLHPYPTDGDKQMLAKQTGLTRNQVSNWFINARVRLWKPMVEEIHNLEMRQVHKQSPHDNGSQHGVHGHAHQPSSQQQQQQRSGKRSEPCDSHLGQCSGVTRNHHHHSNPAASSHGGGFPDDLSQMSHSMQQGQVTFAGYGALPSQSQQHQHQHQHSSMASPQHPHHQHHVGAAGAGNGGGVSLTLGLHQNNRVCFGEPLPANLAHRFGLEDVVSDPYVMGSFGGGQDRHFAKEIGGHLLHDFVG

SEQ ID NO: 82 - protein, 612 aa, Oryza sativa subsp. indica
  misc_feature (1)..(612): Public GI no. 19352105
MSSAAGGGGYGGGQGGGAEHHHHHHGHAGHLLLHHHPQHVAGAAVAAAAAAAGGQMYHVPQHSRREKLRFPPDAGDSPPPHGHGHGHAPQQQQQHGSWPPPPAFYSYASSSSSYSPHSPTLAQAQLVAHGLAPPLPQIPTQNFSLSLSSASSNPPPPQAQPRRQLGGLAQATGPFGPFTGYAAVLGRSRFLGPAEKLFEEICDVGGAASHVDRTISDEGLLDADPMDGVDHDVVDHDLGGADRAAADAGPISGAEQQWKKTKLISMMEEVCKRYRQYYQQVQAVMASFETVAGFSNAAPFAALALRAMAKHFKCLKSMILNQLRNTSNKVAVKDGLNKEIAVFGLAGGSSGGAGLQRANSASAFGQPHNIWRPQRGLPERAVSVLRAWLFEHFLHPYPTDGDKQMLAKQTGLTRNQVSNWFINARVRLWKPMVEEIHNLEMRQMHKHSVVDKGQHSVHHQAQHSSQCSGNPSVPSDSHPGQSSSITRNHNTAASQGFPDELSQMSQSIQGQVSFAYNGLTSQHNIASPHHQHQQVGGVSIGGGNGGVSLTLGLHQNNRVCIAEPLPAALPANLAHRFGLEEVSDAYVMSSFGGQDRHFGKEIGGHLLHDFVG

SEQ ID NO: 83 - protein, 612 aa, Oryza sativa subsp. japonica
  misc_feature (1)..(612): Public GI no. 34908294
MSSAAGGGGYGGGQGGGAEHHHHHHGHAGHLLLHHHPQHVAGAAVAAAAAAAGGQMYHVPQHSRREKLRFPPDAGDSPPPHGHGHGHAPQQQQQHGSWPPPPAFYSYASSSSSYSPHSPTLAQAQLVAHGLAPPLPQIPTQNFSLSLSSASSNPPPPQAQPRRQLGGLAQATGPFGPFTGYAAVLGRSRFLGPAEKLFEEICDVGGAASHVDRTISDEGLLDADPMDGVDHDVVDHDLGGADRAAADAGPISGAEQQWKKTKLISMMEEVCKRYRQYYQQVQAVMASFETVAGFSNAAPFAALALRAMAKHFKCLKSMILNQLRNTSNKVAVKDGLNKEIAVFGLAGGSSGGAGLQRANSASAFGQPHNIWRPQRGLPERAVSVLRAWLFEHFLHPYPTDGDKQMLAKQTGLTRNQVSNWFINARVRLWKPMVEEIHNLEMRQMHKHSVVDKGQHSVHHQAQHSSQCSGNPSVPSDSHPGQSSSITRNHNTAASQGFPDELSQMSQSIQGQVSFAYNGLTSQHNIASPHHQHQQVGGVGIGGGNGGVSLTLGLHQNNRVCIAEPLPAALPANLAHRFGLEEVSDAYVMSSFGGQDRHFGKEIGGHLLHDFVG

SEQ ID NO: 84 - protein, 576 aa, Zea mays subsp. mays
  misc_feature (1)..(576): Ceres CLONE ID no. 398671
MSSAAGGGYGAAGGAEHQHLLLGQASGQLYHVPQHSRREKLRFPPDPADSPPPTAWPAPPPFYSYASSSTSSYSPHSPTLAHTQLVAHALPAGAGAQIPSQNFALSLSSASSNPPPAPRRQLAAGVAAGPYGPFTGYAAVLGRSRFLGPAQKLLEEICDVGGRPPHLDRRSDDEGMLDMDAAGGVDHEMDGGDCATAEAVAVSGAEQQWRKTRLISLMDDVCRRYKQYYQQLQSVISSFETVAGLSNAAPFAFMALRTMSKHFKCLKGMVMSQLRNTSKVIANHGIIAKDDMANFALMGGGAGLLRGNSVNAFSQPHNIWRPQRGLPERAVSVLRSWLFEHFLHPYPTDSDKQMLAKQTGLTRNQVSNWFINARVRLWKPMVEEIHNLEMRQLHKTTSVDQNQLGMQQQNHHSSDGSGRPSSDPSNSQRGQSSGMTRNLSSRAPRHIQDDELSQMPHDMAGQVSFAYSGLPPAHHGXALSHHHHPQQAQAEAVGVGGVAASSGGGVSLTLGLHQNNNNNRAYIAEPLPAALPLSLPHRFGLEDVSDAYVMGPFGGQDRHFSKGIGGGHNLLHDFVG

SEQ ID NO: 85 - DNA, 1883 bp, Arabidopsis thaliana
  misc_feature (1)..(1883): Ceres LOCUS ID no. At1g06110
cttcaaagcaagtggattgatgtgatatctccactgacgtaagggatgacgcacaatcccactatccttcgcaagacccttcctctatataaggaagttcatttcatttggagagaacacgggggactctggcctgcagggccatcaccactgcagtggatgggtctagaggatgctggagatttggttctccacatcgtcttatccaaaatcggccctgaaaacaccgcgagagtggcttgtgtcagtaaacgccttaaggtctccgcctccgaggaatctctctggtctatcttctgctccaatgatcttaatatctctactcctctcgatccccatggagatcctgctccttccttcaaggtcaatttcactcttttcttcttttcacattttctagggttttgattgtttagcgccttagaatttgacctgaatttgagactaccttaaagtaaagatttttctcttaattcatcagagagcatatcaattgtggagggagtcatttagaatgtatccttggaatctggttaaaagagttagactttgttgggacaacctcaaacaatggttgaccttaaacttccctgaagcaaaggcaacactgaggaaaggtgtcacagaagatgatcttcaagaattcgagacttctctcaaagtgaaacttcctttgcccacaaggcttctctaccgtttcgttgatggtcaagagctttcttcccccaatgggcttgatggctctttggggcttataggtggctattccgcttattctcatgacgttaatgtctacttgctacctcttaaggaagtgatgagggagacaaaggaaagtttcatgcgcgacctcggtttctcgagtagattagaccttattgttatggctgcatccgtagttgccagtctgaaaatatttttattagactgcacaaccggacagctttttactgggacaagtaaccgccaattgcttccttgtgtacccgatgctttggttagatcggttcatgataccaacggcgatcagcaacaggatgccatgctgctttggttggaagaacatggccggcggttacaaaccggcactataaatgtccgtcaacagaacaatgtcaagagtatcagtttgttcccggagattcctcccttgtgttctgtctccgtaactaatggtgtgcaggtaaagcaatgtgttattcagttttattgatattcaggtttgtggattgtgggttgaaaagcttgttcatgaataatgcaggtacgtgcttcgtctgtttttatcccggaaatatcgaaccttcgggatcagccaccggcatactggtatgcatattcaatccggatgtctctcatgccagaaggatgcatcttgaatgggacacatcatagctcttgccaactgtattggagacattgggttatccgagctgataatgaagtgatagataatgttaatggagaagctgtcataggaaaggtgtagttttgcttttagttcaagataacaacaatttgtttgttgaaggaccaacagtcgttttccgttttgtgatatgaactctgttttgttatatttgtagtacccgctcttacaagccggggaggaagagtttgtgtatgagagttgttccagttttccgacaactgctggatccattgatggctctttcacctttgtacctggaaggtatatgatgatgtgaagaatcttagaccacatgtcttgtagttttgaaactaaactggaatcttgaccttatgattattttttcttagtttgagagatccaaaagggagtcaattcgaagtcaaagtcgtagagtttcctctggagttaccggactacatcttctgaccagagctctggaaaaaaaaaa

SEQ ID NO: 86 - DNA, 1311 bp, Arabidopsis thaliana
  CDS (1)..(1311)
  misc_feature (1)..(1311): Encodes the peptide sequence given in  SEQ ID NO 87
atgggtctagaggatgctggagatttggttctccacatcgtcttatccaaaatcggccctgaaaacaccgcgagagtggcttgtgtcagtaaacgccttaaggtctccgcctccgaggaatctctctggtctatcttctgctccaatgatcttaatatctctactcctctcgatccccatggagatcctgctccttccttcaagagagcatatcaattgtggagggagtcatttagaatgtatccttggaatctggttaaaagagttagactttgttgggacaacctcaaacaatggttgaccttaaacttccctgaagcaaaggcaacactgaggaaaggtgtcacagaagatgatcttcaagaattcgagacttctctcaaagtgaaacttcctttgcccacaaggcttctctaccgtttcgttgatggtcaagagctttcttcccccaatgggcttgatggctctttggggcttataggtggctattccgcttattctcatgacgttaatgtctacttgctacctcttaaggaagtgatgagggagacaaaggaaagtttcatgcgcgacctcggtttctcgagtagattagaccttattgttatggctgcatccgtagttgccagtctgaaaatatttttattagactgcacaaccggacagctttttactgggacaagtaaccgccaattgcttccttgtgtacccgatgctttggttagatcggttcatgataccaacggcgatcagcaacaggatgccatgctgctttggttggaagaacatggccggcggttacaaaccggcactataaatgtccgtcaacagaacaatgtcaagagtatcagtttgttcccggagattcctcccttgtgttctgtctccgtaactaatggtgtgcaggtacgtgcttcgtctgtttttatcccggaaatatcgaaccttcgggatcagccaccggcatactggtatgcatattcaatccggatgtctctcatgccagaaggatgcatcttgaatgggacacatcatagctcttgccaactgtattggagacattgggttatccgagctgataatgaagtgatagataatgttaatggagaagctgtcataggaaagtacccgctcttacaagccggggaggaagagtttgtgtatgagagttgttccagttttccgacaactgctggatccattgatggctctttcacctttgtacctggaagtttgagagatccaaaagggagtcaattcgaagtcaaagtcgtagagtttcctctggagttaccggactacatcttctga

SEQ ID NO: 87 - protein, 436 aa, Arabidopsis thaliana
  misc_feature (1)..(436): Ceres LOCUS ID no. At1g06110
MGLEDAGDLVLHIVLSKIGPENTARVACVSKRLKVSASEESLWSIFCSNDLNISTPLDPHGDPAPSFKRAYQLWRESFRMYPWNLVKRVRLCWDNLKQWLTLNFPEAKATLRKGVTEDDLQEFETSLKVKLPLPTRLLYRFVDGQELSSPNGLDGSLGLIGGYSAYSHDVNVYLLPLKEVMRETKESFMRDLGFSSRLDLIVMAASVVASLKIFLLDCTTGQLFTGTSNRQLLPCVPDALVRSVHDTNGDQQQDAMLLWLEEHGRRLQTGTINVRQQNNVKSISLFPEIPPLCSVSVTNGVQVRASSVFIPEISNLRDQPPAYWYAYSIRMSLMPEGCILNGTHHSSCQLYWRHWVIRADNEVIDNVNGEAVIGKYPLLQAGEEEFVYESCSSFPTTAGSIDGSFTFVPGSLRDPKGSQFEVKVVEFPLELPDYIF

SEQ ID NO: 88 - DNA, 1332 bp, Populus balsamifera subsp. trichocarpa
  misc_feature (1)..(1332): Ceres ANNOT ID no. 1772685
atggggctggaatcagtgggagatctagctctgaacataatcttaacaaaacttggtccaaaagagacagtacaagtactatgtgtcagcaaaaagttcaaggatttagcttcagaggaatctctctggtcattattttgccgtcaagatcttgatctttctgctcctcttgaccatcatggaaatcatctgccttcttttaaggcaacttataagttatggagagaagcctttcatatgtatccttggccccttgtaaagcgagttaaaagttgttgggacagactcacgagctggttgaccgcgaactttcctgaagttaaggctaccctaggaaagggtgcatcagaaggtgagattcaaaagttggaaagaattttgaaagttaagttgcctcttcccacaagacttctctaccgctttcatgatggtcaacatttctcagacaaaaatctgtcaggtggcatggctggttgtccattgggcctgataggtggctactgtttttataatcactcggttaatgtctacttattatcactacatgaggtaatctctaaaacgcaggaaatagtgcggcacctgaacttacccgatacatccgagtatattgttgtggctgcttcatcctcatacgttggaaagtttttcttcctgaactgttctgatggccaactctatgttgggacccagaattttccaacagatgcagaaatgatgccatgtgtacctcaggcattgattagtccagtccgtgatttcaacagtgaccaacaacaggatgctatgttgttatggttagaagaacatggccgtcgcttgcacaatggcatgatcaaaattctcggcaaaggaaatattaaaagcatctctcagtttccagaagaatctcctctctgttcaactgctgtaaccagtggtgtaaaggttcgtgcttctgctgtttttgtgccagaggctgctgatctggaagatatttctacaaaatacgtgttcgcttattcaatccgcatgtcccttctaccagaaggatgcatcatcaacggaatgcacttcagctcttgccaactgcacctgaggcactgggttatcagtgctaatgatactgctgtatctaatgtcaatgcagaggctgtgataggcaagttcccactcttgtttccaggcgagaaagaatttgtttatgagagttgtacacctctgccaacttctactggctctgttgaaggttctttcacatttgtccctggcagattggcagatccaaaaggaattccatttgaagttgaagtcggtcggtttccgctccaactgccagactacattttctga

SEQ ID NO: 89 - protein, 443 aa, Populus balsamifera subsp. trichocarpa
  misc_feature (1)..(443): Ceres ANNOT ID no. 1772685
MGLESVGDLALNIILTKLGPKETVQVLCVSKKFKDLASEESLWSLFCRQDLDLSAPLDHHGNHLPSFKATYKLWREAFHMYPWPLVKRVKSCWDRLTSWLTANFPEVKATLGKGASEGEIQKLERILKVKLPLPTRLLYRFHDGQHFSDKNLSGGMAGCPLGLIGGYCFYNHSVNVYLLSLHEVISKTQEIVRHLNLPDTSEYIVVAASSSYVGKFFFLNCSDGQLYVGTQNFPTDAEMMPCVPQALISPVRDFNSDQQQDAMLLWLEEHGRRLHNGMIKILGKGNIKSISQFPEESPLCSTAVTSGVKVRASAVFVPEAADLEDISTKYVFAYSIRMSLLPEGCIINGMHFSSCQLHLRHWVISANDTAVSNVNAEAVIGKFPLLFPGEKEFVYESCTPLPTSTGSVEGSFTFVPGRLADPKGIPFEVEVGRFPLQLPDYIF

SEQ ID NO: 90 - protein, 443 aa, Glycine max
  misc_feature (1)..(443): Ceres CLONE ID no. 475075
MGLESVGDLAIHVILSKLGAQDTARVACVSKRFCSSASDDTLWINHCFHELALTQPLDHLGNPLSSFKECYQAWRGAFVMYPWSLVKRVKRCWDKIKTWLTNNFPEAEATLCKGATEADIQELENVLKVKLPLPSRILYRFHNGQEIAKADPETTTYGSSLGLIGGYSFYSHLVNVYLLPIRQIILETKQTRRHLSFLRRSKYVLVAASSTYSRKLFFLNCTNGQLYVGTRDLLTEGDIIPCVPHDLINLHQELNISEQQDAMLLWLEEHGRRLEHGFIKLHDKGNGKSINLFPEEPPLCSMAVTNGVKVRASALVIPELIDLQDDLEKYLFAYSIRLSLEPQGCTINGMSFSSCQLHWRHWIIRANDIVISDVNGEAVIGQYPLLRPGAQEFVYQSRMHLPTPSGSIEGSFTFIPGRLADPKGDPFLATVARFPLQLPDYIF

SEQ ID NO: 91 - protein, 425 aa, Oryza sativa subsp. japonica
  misc_feature (1)..(425): Public GI no. 62733972
MAAPPQPQPEPEPAAGGAGLEALEGLALDTVIAKAGARQAAALACASTRLRDAAGDDALWRRFCADDLALHAPLAPDGRALPSFKDAYKVWLESFGMYPLPLVRRVKIFWSSLKSWLSENFPEAHKTLNKGVSEAQIQSAEDDLGFKLPLPTKLLYRFCNGQLPLSEHHHENMRMAHLGIIGGYVFYDHLINVHLSPLEQIVEETKEFYHDQLYVGTINLQDGEMLPCVPKSLIRPTNTDMPQDGLLLWLEEHLRRLQNGMIKIRMLKTSRYISLFPEASPSCTSAMTNGVKVRASAVFAPEHPESRRPGAKCLYAYSIRLSVPEACMLGGVYYSSCQLYSRHWIIRWRDRVVSDVNGEGVIGKYPLLTTGQEEFVYESCTPLPDSPGSVEGSFTFVPGKLSRPEGKPFEVTVAAFPLEIPEYIF

SEQ ID NO: 92 - protein, 457 aa, Oryza sativa subsp. japonica
  misc_feature (1)..(457): Public GI no. 62733973
MAAPPQPQPEPEPAAGGAGLEALEGLALDTVIAKAGARQAAALACASTRLRDAAGDDALWRRFCADDLALHAPLAPDGRALPSFKDAYKVWLESFGMYPLPLVRRVKIFWSSLKSWLSENFPEAHKTLNKGVSEAQIQSAEDDLGFKLPLPTKLLYRFCNGQLPLSEHHHENMRMAHLGIIGGYVFYDHLINVHLSPLEQIVEETKEFYREFYDQGVFNMTNLIIVATSWYRPKTFFLNCSDDQLYVGTINLQDGEMLPCVPKSLIRPTNTDMPQDGLLLWLEEHLRRLQNGMIKIRMLKTSRYISLFPEASPSCTSAMTNGVKVRASAVFAPEHPESRRPGAKCLYAYSIRLSVPEACMLGGVYYSSCQLYSRHWIIRWRDRVVSDVNGEGVIGKYPLLTTGQEEFVYESCTPLPDSPGSVEGSFTFVPGKLSRPEGKPFEVTVAAFPLEIPEYIF

SEQ ID NO: 93 - DNA, 1686 bp, Panicum virgatum
  misc_feature (1)..(1726): Ceres CLONE ID no. 1797005
actcctccgtctgtctcctccccattccgccaccgcaacaacaaacccctcccggaatccagcccagtccaccggagcgcgccgccccgccgatggcgtcaccgcctccgccggatccagcgccgcctccctcggcggggttggaaagcatggaggggctcgtcatcgacacggtcatttccaaggccggggcgcgccccgccgcggtgctcgcgtgcgccagcacccgcctccgcgccgccgtcgccgacgaatccctttggcgccgcttctgcgcagaggacctggggctcgacgcgcccgtggaccccgacggccagccgctcccgtcgttccaggttgcatataaagtgtggttggagtcttttggtatgtaccctttacctctggtaaagagagtgaaagaattctggagttcaatgaaaacatggttgtctgaaaacttccctgaggcagccaaaacattgtgtaaaggtgttactgaagctcaactaaaatcagcagaggatgaccttggtttcaagcttcctatgcccacaaagctgttgtatcgcttttgcaatgctcaactgccttttagtgaaaaccatgaagctaataaacgcatttccactcatggaataattgggggctatgcgttttatgatcattgggtaaatgtgcatttatcaccacttgagcaaatagttgaagagacaacagagttttgtcgcgagttcccggatgtcttcagtgggcgcaagctcattatagtggcgacttcttggtttcatccaaaaacatttctcctgaattgctcaaatggtgaactatatgttggcacaaacaacttaccattaggtgaaatgctgccttgtgtgcctaaagcgttgataaagccaactgataatgatcttccccaagatggattacttctatggttagaagagcatctcagacgtttacagaacggcatgatcaaaacccgtatgctgacgaagttgaggtatatcagcttatatccagaagcacctccatcatgtacttcagccgtgacaaatggtgttaaggtacgcggatctgctgtctttgtaccagaacatcctggggaccctcagcgaagttgtatgtacacttactcaattcgcctgtcagttccagaggcttgcatgctaggtggcgtgtactattcttcctgccagcttaattcacgccactggaccattcgatcaagggacagggttgtttctgatgtgaggggagaaggtgttattggacagtatcctgtgctgtcacctggtcaggatgagtttgtctacgagagctgcacaccactggccaaagggcctggagctgtggagggctcttttttgtttgtgcctggcaagttgagccggcctgaagggaagccgttcgaggtcatagtggctccgttccctctggaggtgcctgagtacatcttctaatgactgttgagagaaataatgtatctatgtggtagatggttctcccaggtacttattaacttggtggagcaaagtttctttacttgtgatgatcttgttggagtaatgtaaaatatggaaccgtgtgcactttacttgatgtagcagtcgatactaaacaaccacctcagggactgcagcctagtaatctacagatgggcctcagtctctatcctggcaacaaaaaaaaaaaaaaaaaaaa

SEQ ID NO: 94 - protein, 450 aa, Panicum virgatum
  misc_feature (1)..(450): Ceres CLONE ID no. 1797005
MASPPPPDPAPPPSAGLESMEGLVIDTVISKAGARPAAVLACASTRLRAAVADESLWRRFCAEDLGLDAPVDPDGQPLPSFQVAYKVWLESFGMYPLPLVKRVKEFWSSMKTWLSENFPEAAKTLCKGVTEAQLKSAEDDLGFKLPMPTKLLYRFCNAQLPFSENHEANKRISTHGIIGGYAFYDHWVNVHLSPLEQIVEETTEFCREFPDVFSGRKLIIVATSWFHPKTFLLNCSNGELYVGTNNLPLGEMLPCVPKALIKPTDNDLPQDGLLLWLEEHLRRLQNGMIKTRMLTKLRYISLYPEAPPSCTSAVTNGVKVRGSAVFVPEHPGDPQRSCMYTYSIRLSVPEACMLGGVYYSSCQLNSRHWTIRSRDRVVSDVRGEGVIGQYPVLSPGQDEFVYESCTPLAKGPGAVEGSFLFVPGKLSRPEGKPFEVIVAPFPLEVPEYIF

SEQ ID NO: 95 - DNA, 283 bp, Arabidopsis thaliana
  misc_feature (1)..(283): Ceres Promoter PD0901
caaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagctttttaaaatatttctccacgtgcctctttgtgtttgtctatata

SEQ ID NO: 96 - DNA, 999 bp, Arabidopsis thaliana
  misc_feature (1)..(999): Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataggcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaacttatatgagtaatagaaacgatcctaatattaggaattttagagattttctctcatctgtttcttaacttttcaatatttttattttttaaaattgtatgagtttctactaagaaactactgctggagttggtcttagcttcccaatgcttctccacctatatatatgcatatctccttcttaaaac

SEQ ID NO: 97 - DNA, 999 bp, Arabidopsis thaliana
  misc_feature (1)..(982): Ceres Promoter YP0080
aagcggcaatttagtaagaagtactcaaagtatcatttaccaaaagtatatggttttgggaagagttgttagggatgtattctttctaaacagatgatatgacgatgttcttgaaaactaatgttaaagacggaatctctggcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaattacgaaagaaaacaggaaatcaaatcaaaagataaagagaaggtaaaaaaaggcaagaagcactaatgtttaatatttatagttttctccattaaagaaaaagcgatgatgtgtgttctcatcttttgtgaaagtatatatattgcttttgcttttctcaaaagcaaaagactcatccaacaagaacaaaaaaaaaaactaaagctcaatccaaaagacgaagaatgcattggatactacaacttctttttcacttttctttcaaatttacaattatgattttcacaatacagtttattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgtttttatttttctgatgttaca

SEQ ID NO: 98 - DNA, 1015 bp, Arabidopsis thaliana
  misc_feature (1)..(1015): Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcaaaaatagataaactcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 99 - DNA, 1000 bp, Arabidopsis thaliana
  misc_feature (1)..(1000): Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 100 - DNA, 999 bp, Arabidopsis thaliana
  misc_feature (1)..(999): Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaaacttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 101 - DNA, 1000 bp, Arabidopsis thaliana
  misc_feature (1)..(1000): Ceres Promoter YP0388
agaagtattcacgcaccaaggttatatttgtagtgacatattctacaattatcacatttttctcttatgtttcgtagtcgcagatggtcaattttttctataataatttgtccttgaacacaccaaactttagaaacgatgatatataccgtattgtcacgctcacaatgaaacaaacgcgatgaatcgtcatcaccagctaaaagcctaaaacaccatcttagttttcactcagataaaaagattatttgtttccaacctttctattgaattgattagcagtgatgacgtaattagtgatagtttatagtaaaacaaatggaagtggtaataaatttacacaacaaaatatggtaagaatctataaaataagaggttaagagatctcatgttatattaaatgattgaaagaaaaacaaactattggttgatttccatatgtaatagtaagttgtgatgaaagtgatgacgtaattagttgtatttatagtaaaacaaattaaaatggtaaggtaaatttccacaacaaaacttggtaaaaatcttaaaaaaaaaaaaagaggtttagagatcgcatgcgtgtcatcaaaggttctttttcactttaggtctgagtagtgttagactttgattggtgcacgtaagtgtttcgtatcgcgatttaggagaagtacgttttacacgtggacacaatcaacggtcaagatttcgtcgtccagatagaggagcgatacgtcacgccattcaacaatctcctcttcttcattccttcattttgattttgagttttgatctgcccgttcaaaagtctcggtcatctgcccgtaaatataaagatgattatatttatttatatcttctggtgaaagaagctaatataaagcttccatggctaatcttgtttaagcttctcttcttcttctctctcctgtgtctcgttcactagttttttttcgggggagagtgatggagtgtgtttgttgaatagttttgacgatca

SEQ ID NO: 102 - DNA, 2331 bp, Arabidopsis thaliana
  misc_feature (1)..(2331): Genomic sequence for Ceres ANNOT ID 1319615
accgactatatatatatatatatgctgctcttcattaaccccaagaaagaaaaccaaagtgtgaagtccgaatctctctgattctacaattcacaaaaaccggaaaaaaaaaaagacaagtaaagaaagctttgttcagtttacttcaatggaagcaaaacccttagcatcatcatcatctgaaccaaacatgatttctccatcatcaaacattaaaccaaagtaagtttattatggtttcatctacactttttgcgacttctataaacatatacacatgcgtggttttgaaataatgggcgcttatggttctgtcggtgcttgaaacaattttgtcttctcctgactattttttttttgttaataggttatttatacgtgactcttttcttgcagattaaaagatgaagattatatggagctggtgtgtgaaaatgggcagattcttgcaaagattcgaagaccaaagaacaacggttcttttcaaaagcaacgtaggcaatctctcctggatttgtatgagaccgagtacagcgagggtttcaagaaaaacatcaagattcttggagacacacaagttgttccggtgagtcagtctaagccacaacaagataaagaaaccaatgaacaaatgaacaacaataagaagaagctaaagtcctccaaaatcgaatttgagagaaatgtttcgaaaagcaacaaatgtgttgaatcatcaacattaattgatgtttctgctaaaggtccaaagaatgttgaagttactacagctcctcctgatgagcaatctgcagctgttggtagatccacggaattgtattttgcttcttcatcgaagttttctcgaggaacttcgagagatctaagttgttgttctttaaagaggaagtatggagatattgaagaagaagaatcaacctatttaagtaatgtaagagaaattaattagctgcttgtcttagttctttcacatggtagatcattaattgtctttttcttattgcttatgcagaattcagatgatgaatcagatgatgcgaagacacaagttcatgcgagaacaagaaagccggtgactaaaagaaaacgaagcacagaagtccataagttatatgaaagagtgagtttatatgtatgaaaatttcaccttagttagtagaaaaaaaataatgtatgaaaattaatgctaattaaggatttgttaacagaaacgaagagatgaattcaacaagaaaatgcgtgctttgcaggacctactaccaaattgttacaaggtttgagattaacttaaatcacattacattgtttacttagtcttagagagttaattaattttctctttgtggagatttctgaatatctgtaattgttgatgataggatgataaggcttcattgttggatgaggctatcaaatatatgcggacccttcaacttcaagttcaggtattcaaaataatacaccaattcaataaaatttcattgagagataataaaatgattcaggtgttaattatttaaacacagacttgtgtttatatttacatataaatttgttttttaacattaaaatatactttgtaaagtgtttttgttatcaaatattattgcgtttggcttttacctttacgaatgattcttgataaagtcactagaaactagtttcataagagaaaccaaaacaaattataaacgtaattaaacactaatcaataaacaatcaaaagtttattccttatttattttactacaaatttttttacatatatataagattcttaattaacatgctctaataattatttacagatgatgagtatgggaaatggattaataagaccacctacgatgttgccaatgggtcattactctcccatgggtctaggaatgcatatgggtgcagcagcaacaccaacatcaataccgcaattcctgcctatgaatgttcaagcaaccggttttccggggatgaacaatgcaccaccacaaatgctaagctttcttaatcacccaagtggactaattccaaacactcctatcttttctccattggaaaattgctctcagccattcgtggtgccttcgtgtgtttctcagactcaggctacttcttttactcaattcccaaagtctgcgtccgcctcaaacttagaagatgcaatgcaatatagaggaagcaacggttttagttattatcgctcgccaaactaatgatttgtagaaagttgatgttttctccaactaactaactttaagcaaaaaaaaatgatcgtctactctgtgttgttagtctatgggcttttgggccttgattcttggaacgatttgaact

SEQ ID NO: 103 - DNA, 1251 bp, Arabidopsis thaliana
  misc_feature (1)..(1251): Encodes the peptide sequence given in  SEQ ID NO 104
atggaagcaaaacccttagcatcatcatcatctgaaccaaacatgatttctccatcatcaaacattaaaccaaaattaaaagatgaagattatatggagctggtgtgtgaaaatgggcagattcttgcaaagattcgaagaccaaagaacaacggttcttttcaaaagcaacgtaggcaatctctcctggatttgtatgagaccgagtacagcgagggtttcaagaaaaacatcaagattcttggagacacacaagttgttccggtgagtcagtctaagccacaacaagataaagaaaccaatgaacaaatgaacaacaataagaagaagctaaagtcctccaaaatcgaatttgagagaaatgtttcgaaaagcaacaaatgtgttgaatcatcaacattaattgatgtttctgctaaaggtccaaagaatgttgaagttactacagctcctcctgatgagcaatctgcagctgttggtagatccacggaattgtattttgcttcttcatcgaagttttctcgaggaacttcgagagatctaagttgttgttctttaaagaggaagtatggagatattgaagaagaagaatcaacctatttaagtaataattcagatgatgaatcagatgatgcgaagacacaagttcatgcgagaacaagaaagccggtgactaaaagaaaacgaagcacagaagtccataagttatatgaaagaaaacgaagagatgaattcaacaagaaaatgcgtgctttgcaggacctactaccaaattgttacaaggatgataaggcttcattgttggatgaggctatcaaatatatgcggacccttcaacttcaagttcagatgatgagtatgggaaatggattaataagaccacctacgatgttgccaatgggtcattactctcccatgggtctaggaatgcatatgggtgcagcagcaacaccaacatcaataccgcaattcctgcctatgaatgttcaagcaaccggttttccggggatgaacaatgcaccaccacaaatgctaagctttcttaatcacccaagtggactaattccaaacactcctatcttttctccattggaaaattgctctcagccattcgtggtgccttcgtgtgtttctcagactcaggctacttcttttactcaattcccaaagtctgcgtccgcctcaaacttagaagatgcaatgcaatatagaggaagcaacggttttagttattatcgctcgccaaactaa

SEQ ID NO: 104 - protein, 416 aa, Arabidopsis thaliana
  misc_feature (1)..(416): Ceres ANNOT ID 1319615
MEAKPLASSSSEPNMISPSSNIKPKLKDEDYMELVCENGQILAKIRRPKNNGSFQKQRRQSLLDLYETEYSEGFKKNIKILGDTQVVPVSQSKPQQDKETNEQMNNNKKKLKSSKIEFERNVSKSNKCVESSTLIDVSAKGPKNVEVTTAPPDEQSAAVGRSTELYFASSSKFSRGTSRDLSCCSLKRKYGDIEEEESTYLSNNSDDESDDAKTQVHARTRKPVTKRKRSTEVHKLYERKRRDEFNKKMRALQDLLPNCYKDDKASLLDEAIKYMRTLQLQVQMMSMGNGLIRPPTMLPMGHYSPMGLGMHMGAAATPTSIPQFLPMNVQATGFPGMNNAPPQMLSFLNHPSGLIPNTPIFSPLENCSQPFVVPSCVSQTQATSFTQFPKSASASNLEDAMQYRGSNGFSYYRSPN

SEQ ID NO: 105 - DNA, 1343 bp, Arabidopsis thaliana
  misc_feature (1)..(1343): Encodes the peptide sequence given in  SEQ ID NO 106
ccagtcgattggcccgatcggccatggtgacactaacaccatcttctgctagtaccccaaaaacatcttttgatttcatgaagaataacaacagtcacagcagtctctatgtttcttcttcttcttacttgagcagcaaggaagatgctcttgtcacgaccaagaagctcatggaaccaagcaaaacactaaacatgagcattaatccaaaacaagaggagtttggtgatgagaagaaaatggtgaagaaagctcctgaagatccagagattggtgtgtttggagctgagaagtacttcaatggagacatggattcagaccaaggttctagtgttctgtctctgacaaacccagaagttgagagaaccgtcgtcgactcgaagcagagcgcgaagaaatctactggtactccgagtgtccggtctgaatcaagctggaatagtcagagcgtgttgcttcagaacaaactggtgaatagctgcaacagttccttcaaggaaaagaagaacagtaatggtcagattcaaaaggtgaccaataataagaagagttttctcgcaaatttggggtgtaaatgcgcatgctctgatggggattctgtagatgtcgaggagaaaacctcggtcaagagaagcgctgatccgaatatctctgttatcacaatgagatcttctgcggatatgaacacagaactgatcaagattcagaagcaagaggagttatcacagaggaagtctcttgaagtttttggatctccagtggctattgagaagaagagtagtgttgttcagaagaaactaccattgcctccatggaaatcgagaacagaggaggacgacacaaagagtgaagggagtgattcaagctcggatcttttcgagatagagggtcttacagggaaccctaaaccttttcttacgaggcaaggaagtgatccagcttcacctacgtgttatgcgccaagtgaagtaagcgtagagtggagcatagtgacagcaagtgcagcagatttctctgttatgtcagaatgtgcaacaagtcctgtaagaagaaaccgacctactcagattcctcgaatccctattaccgctaaatcagcaccgcagagacggaaatcgagtagcagcagcggagggaatggtttcttgatgagctgcaagagtcataaatctgttatggtttctggtgatttagacagaagaagcagcatgaacaagacacaaccgagttacgttcctagattcccaatggagactactaaacctaagagtttcgaaacacgaagaaggatcagcaacagctcgatttctcacacacaatcatctcttctttatagtcagtga

SEQ ID NO: 106 - protein, 439 aa, Arabidopsis thaliana
  misc_feature (1)..(439): Ceres ME18596
MVTLTPSSASTPKTSFDFMKNNNSHSSLYVSSSSYLSSKEDALVTTKKLMEPSKTLNMSINPKQEEFGDEKKMVKKAPEDPEIGVFGAEKYFNGDMDSDQGSSVLSLTNPEVERTVVDSKQSAKKSTGTPSVRSESSWNSQSVLLQNKLVNSCNSSFKEKKNSNGQIQKVTNNKKSFLANLGCKCACSDGDSVDVEEKTSVKRSADPNISVITMRSSADMNTELIKIQKQEELSQRKSLEVFGSPVAIEKKSSVVQKKLPLPPWKSRTEEDDTKSEGSDSSSDLFEIEGLTGNPKPFLTRQGSDPASPTCYAPSEVSVEWSIVTASAADFSVMSECATSPVRRNRPTQIPRIPITAKSAPQRRKSSSSSGGNGFLMSCKSHKSVMVSGDLDRRSSMNKTQPSYVPRFPMETTKPKSFETRRRISNSSISHTQSSLLYSQ

SEQ ID NO: 107 - DNA, 2009 bp, Arabidopsis thaliana
  misc_feature (1)..(2009): Ceres ANNOT ID 508164
atggacaacaacaacaacaacaacacttttagttctctggataatgtcatgactaaccaaaatcctcttctcatggattttataccttcaagagaagattcaacttcattctcaacaatgcttccatggaataccatcagatcagatcctctacaaatgggtggctttgatattttcaattctatgctgactaacaaatacttatcatcttctccacggtctatcgatgttcaagataaccgcaatgttgagttcatggctcctcctcctcatcctcctccacttcatcctttggatcatttaagacactatgatgattcctcaaacaacatgtggggttttgaagcaaatagtgagtttcaggcattttcaggtgtagttggtccaagtgaaccaatgatgtctacattcggtgaagaagatttcccgtttctaatttcgaataaaagaaacaatgagctttcattgagtcttgcatcagatgtttctgatgaatgctcggagataagtctttgtgcagctacaagattagcctcagagcaagcttcttgcagcagcaaagacatttctaataacgttgttactcaaggtttctctcaacttatatttggctcaaaataccttcactctgttcaagaaatactatctcatttcgccgcatactcgctcgattattcatctcgaggaaccgagtcaggagctgctagttcagcctttacttcacgttttgagaatataactgagtttcttgatggtgattctaataactcggaggcgggtttcggatctacatttcaaaggagagcattagaagcaaagaaaacccatctcttggatcttcttcaaatggtatgtaatatattcattcactttttttgcataagttaaaaaaatggtttgatatatatgatgaagttttatgagttgatatttctcttcaggtggatgatcgatatagtcattgcgtagatgagattcatacggttatatcagcgttccatgctgcaaccgagttagatccacagttacacacccggtttgccctccaaaccgtttccttcttatacaagaacctgagagagagaatctgcaagaagataatctctatgggatctgtattggagagaggcaaagacaagactcaagaaacctctatgttccaccagcattgccttcttcagcagctgaaacgaaagaaccatcagatttggagacctcaacgaggtttgcctgagaaatctgtttcggttctacggaattggatgttccaaaacttccttcacccgtaacaatccttactcttcttaactatctactttatgatgatgacattgttaactcgattttataagcaaagttatgttttatttaatgcagttacccgaaagattcggagaaacatcttctagctatacgaagtggcttgacaagaagtcaggtaatgtctcatgtttgttttcttatgcaatgactcattgataacaagctttgttttcttatgcaatgactcattgataagatctttgttttcttatgcaatgacttattgataacatctttgctcacaatgatagcaaatatggtattcaaatgagtaatatatatcttgacatactagtcatgtctgaaaaaccggtattggttattttaagatgagattagggctaggaaaaaatcaagcttttctcttgaactacaaatgagactagattatgtattcttgaattcacaataatgttactcaagtcctaggattgtagtcgcacatatgttttgttactgaagtaagttaaagcgggcaaatgatatagagtgtgtctctttgttgtgtgtgattaaaggtatcaaactggtttataaatgcgcgggttaggctatggaagccgatgatagaagagatgtatgcggaaatgaacaagaggaagctcaataacagtcacattcaacccaacggaccaactcttcgaatgccaaaatctgttatgatgagccaagcaatgcataaataa

SEQ ID NO: 108 - DNA, 1422 bp, Arabidopsis thaliana
  misc_feature (1)..(1422): Ceres ANNOT ID 508164
atggacaacaacaacaacaacaacacttttagttctctggataatgtcatgactaaccaaaatcctcttctcatggattttataccttcaagagaagattcaacttcattctcaacaatgcttccatggaataccatcagatcagatcctctacaaatgggtggctttgatattttcaattctatgctgactaacaaatacttatcatcttctccacggtctatcgatgttcaagataaccgcaatgttgagttcatggctcctcctcctcatcctcctccacttcatcctttggatcatttaagacactatgatgattcctcaaacaacatgtggggttttgaagcaaatagtgagtttcaggcattttcaggtgtagttggtccaagtgaaccaatgatgtctacattcggtgaagaagatttcccgtttctaatttcgaataaaagaaacaatgagctttcattgagtcttgcatcagatgtttctgatgaatgctcggagataagtctttgtgcagctacaagattagcctcagagcaagcttcttgcagcagcaaagacatttctaataacgttgttactcaaggtttctctcaacttatatttggctcaaaataccttcactctgttcaagaaatactatctcatttcgccgcatactcgctcgattattcatctcgaggaaccgagtcaggagctgctagttcagcctttacttcacgttttgagaatataactgagtttcttgatggtgattctaataactcggaggcgggtttcggatctacatttcaaaggagagcattagaagcaaagaaaacccatctcttggatcttcttcaaatggtggatgatcgatatagtcattgcgtagatgagattcatacggttatatcagcgttccatgctgcaaccgagttagatccacagttacacacccggtttgccctccaaaccgtttccttcttatacaagaacctgagagagagaatctgcaagaagataatctctatgggatctgtattggagagaggcaaagacaagactcaagaaacctctatgttccaccagcattgccttcttcagcagctgaaacgaaagaaccatcagatttggagacctcaacgaggtttgcctgagaaatctgtttcggttctacggaattggatgttccaaaacttccttcacccttacccgaaagattcggagaaacatcttctagctatacgaagtggcttgacaagaagtcaggtatcaaactggtttataaatgcgcgggttaggctatggaagccgatgatagaagagatgtatgcggaaatgaacaagaggaagctcaataacagtcacattcaacccaacggaccaactcttcgaatgccaaaatctgttatgatgagccaagcaatgcataaataa

SEQ ID NO: 109 - protein, 473 aa, Arabidopsis thaliana
  misc_feature (1)..(473): Ceres ANNOT ID 508164
MDNNNNNNTFSSLDNVMTNQNPLLMDFIPSREDSTSFSTMLPWNTIRSDPLQMGGFDIFNSMLTNKYLSSSPRSIDVQDNRNVEFMAPPPHPPPLHPLDHLRHYDDSSNNMWGFEANSEFQAFSGVVGPSEPMMSTFGEEDFPFLISNKRNNELSLSLASDVSDECSEISLCAATRLASEQASCSSKDISNNVVTQGFSQLIFGSKYLHSVQEILSHFAAYSLDYSSRGTESGAASSAFTSRFENITEFLDGDSNNSEAGFGSTFQRRALEAKKTHLLDLLQMVDDRYSHCVDEIHTVISAFHAATELDPQLHTRFALQTVSFLYKNLRERICKKIISMGSVLERGKDKTQETSMFHQHCLLQQLKRKNHQIWRPQRGLPEKSVSVLRNWMFQNFLHPYPKDSEKHLLAIRSGLTRSQVSNWFINARVRLWKPMIEEMYAEMNKRKLNNSHIQPNGPTLRMPKSVMMSQAMHK

SEQ ID NO: 110 - DNA, 1728 bp, Populus balsamifera subsp. trichocarpa
  misc_feature (1)..(1728): Ceres ANNOT ID no.1477240
atggcaggaaggaattctgttaccatggactgttcatcacaaagaacccccaactcacttgtccagttggattcatttaaccttaaccaccataatcaaacattggctggatttaccatgcttccaacattgcaaggagaacctataagtgatctccatgcaaacattcactcagcaaatcgatcttcatttatgaactcagatgcattagttgcatctcttggaaggaatgttgtgggagatactttacctggctgttcacgttctgctggtaaccctccatttgaagagcattttgggagtggaatccctaattatgcacttgctactcttgtggctacaagaagtggtcttcaagaaactctgaacaacttggcaatttcaggaccatcaagctaccctttggaagagtcaaggtcatttgtttcaaatgattgcaccaatgctttgaattcatcatttgcgtcatctttgaattatggatgtggtgaagtatttggtagtacgaatggtaaggaggattttgacaggtttcctgctcccatagagctcagtggacgaacacctttaagagcagggtttcaaccacattcgtccgttggaaacctgcaaccaaatggctggataacatcaaatggtgtgaatgtgagtgcagatgaatgttttgcatctggtaaacttgctaatgagctctctttaagccttgccacatctcagccttctgtcatggatagcagaagtatccctgatcaaagctcggagatcgctcttaatcatgtagcacgccacttctcgaaagaaacaaggttaggctctgaacaaacttcttgtagcagtaaggagctttctttgagttgcagttcttacaaaactggtcaatcctcacaagtattattaggatccagatatcttcatgtaatacaggaaatacttgctcaaattgcaagctattcactggagaatctagaccaggggttcaagactggagcaagtacactattctcttcaagttacgcaatggagggagggatgccgctgatgggctttgataaatctcctgatgggagtgatagacttgatattcaaatggatccagcactgcagaaacgggcactggaagcaaagagaacacaattgctaactctactgcaagtggttgatgaacgatatagccaatgcttggatgaaatacacacagttatttcagcattccatgctgcaactgagttggacccacagattcacacacgtttttctcttcaaacaatatctttcttgtacaaacgccttagggagcggatcagcaaccaaatcctagcaatgggagctcatttggatagtggagacaccatagagacagaaggatcttttggaacttcatacttacaaaagcaatggactctccagcagctgaagaaaaatgaccatcaactatggagaccccagagaggcttaccagaaagatctgtctcagttctgcgtgcatggatgtttcagaactttcttcacccgtaccctaaagatgcagagaagcatttgcttgcggcaaaaagcggactaacaagaagccaggtatcaaattggtttataaatgctcgtgttcgtctatggaaaccaatgatagaggagatgtatgccgagatgaacagaagaaaggctcaccaaaacgaagagggaaccaacagcaatcacagaattagcatatcagcaatccaagatttaatgtga

SEQ ID NO: 111 - protein, 536 aa, Populus balsamifera subsp. trichocarpa
  misc_feature (1)..(536): Ceres ANNOT ID no. 1477240
MLPTLQGEPISDLHANIHSANRSSFMNSDALVASLGRNVVGDTLPGCSRSAGNPPFEEHFGSGIPNYALATLVATRSGLQETLNNLAISGPSSYPLEESRSFVSNDCTNALNSSFASSLNYGCGEVFGSTNGKEDFDRFPAPIELSGRTPLRAGFQPHSSVGNLQPNGWITSNGVNVSADECFASGKLANELSLSLATSQPSVMDSRSIPDQSSEIALNHVARHFSKETRLGSEQTSCSSKELSLSCSSYKTGQSSQVLLGSRYLHVIQEILAQIASYSLENLDQGFKTGASTLFSSSYAMEGGMPLMGFDKSPDGSDRLDIQMDPALQKRALEAKRTQLLTLLQVVDERYSQCLDEIHTVISAFHAATELDPQIHTRFSLQTISFLYKRLRERISNQILAMGAHLDSGDTIETEGSFGTSYLQKQWTLQQLKKNDHQLWRPQRGLPERSVSVLRAWMFQNFLHPYPKDAEKHLLAAKSGLTRSQVSNWFINARVRLWKPMIEEMYAEMNRRKAHQNEEGTNSNHRISISAIQDLM

SEQ ID NO: 112 - DNA, 702 bp, Panicum virgatum
  misc_feature (1)..(702): Ceres CLONE ID no.1811587
atgtttctgatgttctgtgtctttgtgagtgagagagagccccaggccagataaatctctggacccagcagtgtcaagatcacggagagagaggggcctgctcctggaggctttgatgctggatgcttgtgtgctgaaatcgcatatattttccgggatttcctgacctgcgactacaatggcgagtaatccgtcgacgttcgcgccaatcggggtggatgccatgactggcggctacttcatggcgggcggtggaggcggcggcatgatgagcgccgatgcgccgcacttccaccccagcgtgcttcttgagcacggcgggtttggcttcggcttcggcgacgcggcggtcggcgccgccgcggcgccgtcggacctgggcgtgaactacgcggcaaacaacgccatgctggcctccttcgccagccacctcttcgctaccgctgccgcgccgcccctggatgatcacttcggcgggaggacgccgccggagatggacgaggggtatggagctggcagcgatggaagctccgcggcgagcctgcagtgccccggccactcgggcgccatggcggtttggtcgtcgtcatcgtcgtccaagaagccggccggcacttggatcaccgccggcgggtccagagccgtctcggtccacgagccgtactacctcgccggagtccccgacgtcgccggtttcactac

SEQ ID NO: 113 - protein, 525 aa, Panicum virgatum
  misc_feature (1)..(525): Ceres CLONE ID no. 1811587
MASNPSTFAPIGVDAMTGGYFMAGGGGGGMMSADAPHFHPSVLLEHGGFGFGFGDAAVGAAAAPSDLGVNYAANNAMLASFASHLFATAAAPPLDDHFGGRTPPEMDEGYGAGSDGSSAASLQCPGHSGAMAVWSSSSSSKKPAGTWITAGGSRAVSVHEPYYLAGVPDVAGFHYPLIAAAAANAPASSELSLTLCSKSFPDSALNGAEQCSSGASRSALTELPQARPRPAHFSVVVARSRYAAVAQAVLNDVAGHLLDGVADVAADSCSGGARPSSGSVGARAPTVVSSNRLLASSQDGGEAQRVRSHLLKMLHLMDEKYNQCLNEIQSTTAKFNALMQPGAAGVVSSGSIRAAFAHRAVSAVYRGLRQRIAGEIIAAASRAAGCWGESSSSVTAAGDAERSWESAFIRKHWAAQQLRRGEQQCWRPQRGLPEKSVAVLKAWMFENFLHPYPKDHKKDVLASRSGLTRNQVSNWFINARVRLWKPMIEEMYEDQKKSSGGQGAAMEPHTSKRRIREVEEGQGTP

SEQ ID NO: 114 - protein, 535 aa, Zea mays
  misc_feature (1)..(535): Ceres CLONE ID no. 1580361
MASDPSTFSPIGVDAMGGGYFMAGSSSYGGGGIMSAEVPHFHHPGVLLDQGGFGFGGLGNAAVVGGAATAADLGAHYAANNIVLASFASQLLANAPAPPRDDRAGGRTPPDEMDEELYGVAGCDSRVAASLRCPSQSGAMAVWSSPSSSKKPYGIWTSAGGPAHEPYHLAAAGLSDAGGLRYPLAACSGGNASAAAASELSLTLCSNSIASSDSALNATEQCSSGASRSALTELPRARSRMALHFAAVVARSRYAAVVQDLLNDVVGHMLDGVADVTDDSCSGIGSVGAPSAVSSNRFMASTEDAGARWGQAQRVRSNLLKTLQLMDEKYNQCLDEIQSTTARFNTLMHSAPGGGGICAPFAHRAVSAMYRGLRRRLAGEIMAAASRASCWGESSSSVTVAAGGDVERSWESAFIQKHWSAQQLRRTEQQCWRPQRGLPEKSVAVLKAWMFENFLHPYPKDHEKDVLAARSGLTRNQVSNWFINARVRLWKPMIEEMYQDLKRSSGAGGGGHGPAMEPQQHLSKRRICELEDGGQ

SEQ ID NO: 115 - DNA, 721 bp, Gossypium hirsutum
  misc_feature (1)..(721): Ceres CLONE ID no.1943506
catgttcttcttaagccccacaagcttaccccaagcaccaccgcctaaccatcatcagcgtttccttggtctccctctcccaacaccatccaccgccatcaaaccgctcaattccgacgatccccaccgtccatccccccttcaaggtgtagtccccggtttccactctaacttatggggttcaagtgttgatcatcagaattccccaggaagtagccatccgcaggttgtgtcggcagtagctacaggagataactctggtgggtcccatgatgttgcgtcgcagtcggggtttcaacggccggtggtgtccccaaggcaaggtttgtccctaagtctatcatcccaacaagtgggttataggtcaagtaataatgttgaaacagacatacaaggacaacctcaagttcctactatgtcactgggtgaagatgtgaggatatcagggaactcgccgtcgtcggtttccgttgtttcgaatgggatttctggggctcagagtgtggttttggggtcaaagtacttgagagctgcacaagagcttcttgatgaagtcgttaatgtggggaaagggataaagacggacgtgtctgaggggaccaaggaagagaagataaaggtgaacaaagaatcggtggctggagagggttcaagcgctggtgaaaatggggctaaacgtggagctgagctcaccacagctnaaagacaggagctgcagatg

SEQ ID NO: 116 - protein, 314 aa, Gossypium hirsutum
  misc_feature (1)..(314): Ceres CLONE ID no. 1943506
MSLGEDVRISGNSPSSVSVVSNGISGAQSVVLGSKYLRAAQELLDEVVNVGKGIKTDVSEGTKEEKIKVNKESVAGEGSSAGENGAKRGAELTTAQRQELQMKKAKLVSMLDEVEQRYRQYHHQMHIVVSSFEQVAGLGAAKSYTALALKTISKQFRCLKDAISGQMKATSKSLGEEDCLGAKVEGSRLRYVDHQLRQQRTLQQLGMIQHNAWRPQRGLPERAVSVLRAWLFEHFLHPYPKDSDKHMLAKQTGLTRGQVSNWFINARVRLWKPMVEEMYLEEVKEQERNGSEVKANKSVQKESESSSSAHQENV

SEQ ID NO: 117 - protein, 476 aa, Solanum demissum
  misc_feature: Public GI ID no.48057594
MAMVKLEATKSTSTNLLDPSFSSYLINGTEEAIVFNLESSRDLSKKVDDGEIDIFSAEKYFNEGVDEVNNVTQNKLKIHDDQPVAVADIVSLQQKIRPLTPSIHSESSWNSRSALLQKVSRNHHYHQQHHQPPWPTKTNNKSYGKKFLARFGCNCYCKDKNSVEIDDQLGEKSFNRVKYSKSKQNIIKTRSSESSTIGANHQDLHFKKIDELGVLGLKPDERFAVPVFDPKGGIQMKKESEEEESRKSLEVFGFPITEKERSKMSLEKNIGMLTWDAIVPKAEEIDIINIGASSNGTYEEDYAESDASSDLFEIESFPNNNTANPSLVRQGSDSMSCYAPSEVSIDWSVFTASAADFSIMSDIEEVKIPSIRTTSNSNRSVSQNGRDKAKRRSGILLGCNSHKAVGVVGDAYKVSEKSSIEMHQRNFKTYEPIMPMTRFHAESKVNRFDGGNRKHEFTTRSFATTYTRRPADFLYI

SEQ ID NO: 118 - DNA, 3000 bp, Arabidopsis thaliana
  misc_feature (1)..(3000): Ceres Promoter PR0924
atctataacgagttaacatgttgccagtttgaatcaagaagcttggatgatgaatgaatggatcggtttgtggtacaattcttaaaattgtagtagaggagacagagaaaaaacatgataagactttggtatttacaacttgacggagacaagacagtaagccaaatctgtcacaaaaacactcaaactcttttctcagtgttttgagtttaaagagagacttattcacttcccctttcgtaacacttatttgtctcccaaccaaacagtttctgtcctttcccttgtcctcccacgtgcatctttatatctcatgacttttcgtttctagatcttgaataatgtcttagtggattaggtttgttgtcggtaaattaggtgaccgtttttttcttatatttggaagatcgcgggatgaagcagatactgagtttcagggcatacacacctaatttgaaaatcattgttagtccaatttcactttaatcttgtttacaaaaaaattgatctgaaaatgttgatgggataagtaaaaatgtaagttttgctagtagtcatgatataataatagcaaaaccagatcaattttgagcaaaaggaagaaacaaaaaacagatcgatcccacgagcaagactaagtgtaaagtggttcccacaagagccatatggatatggtccttcaacttttaaagcccattacttcagtggtcgacccgacattacgccacgagtagtcacgcacgcacgactccgttcacgtgacattcacgttgatatttccccctctactctcttctgcttggttgatctaaaaaacatgaagagaccaacctaatttcatattaatatatgatatagacttcatactcaacagtcactttcgtaatccaaatccatatcttacgaaattagttcttaataaaggttgtggattaagttataatattgtgttaagagttaagacacagcatataaccttgtaccaacagtgctttattcttaaatggaaacaaaacatatgtcaatgtcaagcatacagctaaaatatcattatctaatattaagagtaaaacaagataattaaaaattgaaacaacaccatatttttatagctttacttatcgtatttttctagtcttcatggtaattgtgttgctttattttgtttataaatgaatttggttcgaccagatagtctaatatcagtttttaaacactggttttaataaaatcatatgtcggcaattcaacctgttacgttgtatgattgtatcctagtcaaataggggaggaggtactagtcgtttcaattagtttacgtaatcaatccaaagaaactataagctataaagatcctcaatttgttggttacaataaaaacaacagttgtcaaaatttatgtttataaaaagtaataactatgttccttcccatatagagcaaagtacctcaggataggcaaaccgtacttaatagcccttattcataatttgatccaactcttccccacaaaattgcaactgatgaagtcaatacttgtatagtgagtcaagctataaatgtctagtgatagttttgtctcttaaaaggttaacaaaagttatgacaagctgaaaaatcagagtttgctaggagtattacttacagttatcagtttaagtatcacatttatagtattgtatacaatgattcttaaattccaccttttccgtgcgaaaccaaattttctattggaaacatagaatgtaaacaaaaatatgggacgttgtccgttccaacattaaccaaacttgtctattactaatattcgtgttggtttgatgttggatgtctaaattcgttgaatcatgtgtctcttgacgaaatatgcatcttcttatttcttagtatagatgcactttatcattcttttagtacatgcttaatttttttttttaaaatatgttgattgtcatattgccaaaagtatgaattaaagacgcacatctaacacaagttagcagccgtaaatccttccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 119 - DNA, 2216 bp, Gossypium hirsutum
  misc_feature: Ceres CLONE ID no.1924114
gtgagcttagcggagaaaagagtcagaaaaatctgcaacacagtttagtttcaattattacagcaaaaaccagtcatcttcccattctccacccctattatcactaccaccacacttctctcatagtcattcgccataaaagaaacagtaacaaagattttgtttttttgagtttgagtgtttgttttagtttgaaagtgagtgacagacattattagattattagaatatgtttatatatatatatatatagtttctgtaataaataaataaaaaaaaagattcgtcaaagtgaaaaacgtgcatcgataccccaaaaatctcagttccttgaacgtatcatactctgctaacttgcgccacttctctctcttttctctgccattttaatattttcccagtttcagaagattacaatcatttcatcgtttaaattaatcaaataaagctttttcactttcactgttatttagtttttagcttatagagaagttgtttgttggttttttttttcttttcttttgcttctttttttagggctatggcggagggttttgagccctaccatgtcccacaacaaagcagaagagataagctaagaatcatgggtcaaaatgaaccaacaacgggtgttcctctttcgggttgttcgggtttactccctttttatgacccttctttcctttcttccgatttgctaacttgcgccgctgccgccgccggaagccatgaatatcaccaccctcctccgtcgggtacaaaagatggcgtgaacttcacaggctttgttggtggggttttcaactcttctccttctttggatcacttgaaccctagctccattcatgatgtggacaacaacaacaacaacaaccagtttctttataccccacaaaacctgtcttatgataataataacggtggtggtgggggtggtgaagtggtggtttataagcctgaacctttatctctttcattatcttctcattatacccaccaaaactctagtatctatactgatatggttccagccatttttagtggtgctaatggttcaacatcgaactcagtcccactcggtcctttcactggctatgcttccattttgaaaggttcaaggtttttaaggcctgcacaacagttattagaagagctttgtgatgttggtaagggaatttacactgaaaaagcatctctcatggagcttcctccattgcaaaatccccacactaaccctcttgacggcggagatagcagcggaagcggcggcggaggtgatggtcaaaggaaaaaatcaacactaatttcaattctcgacgaggtttacaagaggtacaagcaatactatcagcagatgcaatccgttgtcgcttcgtttgaatgtgtcgccggactagggaatgcagctccatttgcaaacttggctatgaaagctatgtctaaacatttcaggtacttgaagaacgcaatcaccgaacagcttcagtttactaataaagctcatgctcagataagccccgggaaaaacgaaggtccgaggttcggaaatggtgatggaagcttttataaccgagctgttcaaaactccgggttccttcaaaaccaaccagtttggcgtcctcaacgaggccttcccgaacgtgcagtgaccgtacttagagcatggctatttgaacactttctacacccttatccgaccgacacggacaagctaatgttggcgaaacaaaccggtctttcacgtaaccaggtctcgaattggtttatcaatgcaagagttcggctttggaaaccaatggtggaagaaatacacatgctcgaaacacggcaaaaggacgagagaaatgccaacaagtcaggcgatgaaaacccatcgacatcagcccaaagggttgaagacaagacgccatcaaagcgtactagaaatgaacttcccaatgtacctgtgggaaatgaacagccaaacatgtcaacctcctacaacagcttttctacccacccgcatagcagcagtgtttccttaacactcggtcttcatcagaacaacagtatcggattatcggagtcctttcccataaacgcggctcaatgtttcggtcttggcatcgagggaaacagtgagggatacgttattggtggtcggcatttcggaagggatgttgtcggagggcagcttttgcatgattttgtgggttgaaaaaaaaaatgttacaagc

SEQ ID NO: 120 - protein, 551 aa, Gossypium hirsutum
  misc_feature: Ceres CLONE ID no.1924114
MAEGFEPYHVPQQSRRDKLRIMGQNEPTTGVPLSGCSGLLPFYDPSFLSSDLLTCAAAAAGSHEYHHPPPSGTKDGVNFTGFVGGVFNSSPSLDHLNPSSIHDVDNNNNNNQFLYTPQNLSYDNNNGGGGGGEVVVYKPEPLSLSLSSHYTHQNSSIYTDMVPAIFSGANGSTSNSVPLGPFTGYASILKGSRFLRPAQQLLEELCDVGKGIYTEKASLMELPPLQNPHTNPLDGGDSSGSGGGGDGQRKKSTLISILDEVYKRYKQYYQQMQSVVASFECVAGLGNAAPFANLAMKAMSKHFRYLKNAITEQLQFTNKAHAQISPGKNEGPRFGNGDGSFYNRAVQNSGFLQNQPVWRPQRGLPERAVTVLRAWLFEHFLHPYPTDTDKLMLAKQTGLSRNQVSNWFINARVRLWKPMVEEIHMLETRQKDERNANKSGDENPSTSAQRVEDKTPSKRTRNELPNVPVGNEQPNMSTSYNSFSTHPHSSSVSLTLGLHQNNSIGLSESFPINAAQCFGLGIEGNSEGYVIGGRHFGRDVVGGQLLHDFVG

SEQ ID NO: 121 - protein, 575 aa, Arabidopsis thaliana
  misc_feature: Public GI ID no.15241667
MADAYEPYHVLQQSRRDKLRIPSLDSHFHFHPPPPPSSGGGGGVFPLADSDFLAAGGFHSNNNNNHISNPSYSNFMGFLGGPSSSSSTAVAVAGDHSFNAGLSSGDVLVFKPEPLSLSLSSHPRLAYDLVVPGVVNSGFCRSAGEANAAAVTIASRSSGPLGPFTGYASILKGSRFLKPAQMLLDEFCNVGRGIYTDKVIDDDDSSLLFDPTVENLCGVSDGGGGDNGKKKSKLISMLDEVYKRYKQYYEQLQAVMGSFECVAGLGHAAPYANLALKALSKHFKCLKNAITDQLQFSHNNKIQQQQQCGHPMNSENKTDSLRFGGSDSSRGLCSAGQRHGFPDHHAPVWRPHRGLPERAVTVLRAWLFDHFLHPYPTDTDKLMLAKQTGLSRNQVSNWFINARVRVWKPMVEEIHMLETRQSQRSSSSSWRDERTSTTVFPDNSNNNPSSSSAQQRPNNSSPPRRARNDDVHGTNNNNSYVNSGSGGGSAVGFSYGIGSSNVPVMNSSTNGGVSLTLGLHHQIGLPEPFPMTTAQRFGLDGGSGDGGGGYEGQNRQFGRDFIGGSNHQFLHDFVG

SEQ ID NO: 122 - protein, 575 aa, Arabidopsis thaliana
  misc_feature: Public GI ID no.23397273
MADAYEPYHVLQQGRRDKLRIPSLDSHFHFHPPPPPSSGGGGGVFPLADSDFLAAGGFHSNNNNNHISNPSYSNFMGFLGGPSSSSSTAVAVAGDHSFNAGLSSGDVLVFKPEPLSLSLSSHPRLAYDLVVPGVVNSGFCRSAGEANAAAVTIASRSSGPLGPFTGYASILKGSRFLKPAQMLLDEFCNVGRGIYTDKVIDDDDSSLLFDPTVENLCGVSDGGGGDNGKKKSKLISMLDEVYKRYKQYYEQLQAVMGSFECVAGLGHAAPYANLALKALSKHFKCLKNAITDQLQFSHNNKIQQQQQCGHPMNSENKTDSLRFGGSDSSRGLCSAGQRHGFPDHHAPVWRPHRGLPERAVTVLRAWLFDHFLHPYPTDTDKLMLAKQTGLSRNQVSNWFINARVRVWKPMVEEIHMLETRQSQRSSSSSWRDERTSTTVFPDNSNNNPSSSSAQQRPNNSSPPRRARNDDVHGTNNNNSYVNSGSGGGSAVGFSYGIGSSNVPVMNSSTNGGVSLTLGLHHQIGLPEPFPMTTAQRFGLDGGSGDGGGGYEGQNRQFGRDFIGGSNHQFLHDFVG

SEQ ID NO: 123 - DNA, 1713 bp, Sorghum bicolor
  misc_feature: Ceres ANNOT ID no.6039739
atgtcttcggcggcgggcggtggtggatacggggccggcggcggcgccgagcaccagcacctgttgctggggcaggcggcggggcagctctaccacgtgccgcagcacagccgccgcgagaagctgcggttcccgcccgacccggccgactcgcccccgcccactgcctggccggcgcccccacccttctactcctacgcgtcctcgtcgacgtcgtcctactcgccgcatagcccaacgccgctggcgcacgcgcagctcgtggcgcacgcgctacctgcaggcgccggggcccagatcccgagccagaacttcgcgctctcgctctcctcggcgtcctcgaaccctccgcccgcgccgcggaggcagctcgccgcgggggtagccacggggccgtacggcccgttcacgggctacgcggccgtcctcggccggtccaggttcctcggccccgcgcagaagctgctcgaggagatctgcgacgtgggcggacggcccccgcacctggaccggcgctccgacgagggcatgctcgacatggacgccatggacgtggttggggacgtggaccatgacatggacggcggtgaccgcgctaccgctgaggcggtcgctgtctccggcgccgagcagcagtggaggaagaccaggctcatctccctcatggaggacgtttgcaggcgatacaagcagtactaccagcagctgcaatctgtgatctcctcgtttgagactgttgcggggctgagcaacgccgccccctttgcttccatggctcttcggacaatgtcgaagcatttcaagtgtctcaaggaaatgataatgagccaattaaggaacacaagcaaggttgtcgccaatgatggtattggcaaagacgatatggctaactttgcgcttatgggtggtggagcaggcctcctgaggggaaacaatgtgaacgcattcggccagcctcacaacatttggaggcctcaaagagggcttcctgagcgtgcagtgtctgtgcttcgctcgtggctgttcgagcacttcttacatccgtatccaactgatagtgacaagcagatgctggctaaacagacaggcttaacaaggaaccaggtgtcaaattggtttatcaatgcaagggtgcggctctggaagcccatggtggaagaaatccacaacctggagatgcggcaggtgcagaagaacacatcggtggacaagaatcaacttggcatgcagcagattcagcactcgacggacagcagtgggaagctttctgacccttcaaactctcagcgtgggcaaagcagcggcatgacaagaaacctcagctctccggcatcacgccatatccaagacgagctctcccagatgccccacgacatgccaggccaggtgagctttgcttacaatgggctccccacacaccacggtcttgcgctgtcacacccccagcaagctgaagcagtcagcgccggcattggccttggcggcgtggctgccggcggtggcggcgtttccctcacactcggccttcaccagaacaacaggacttacattgccgagccacttccggccgcgctcccgctcaacctcccgcaccgttttgggctggaggacgtgagcgacgcctacgtgatggggtcattcggcggcggccaggaccgtcatttcaccaagggaataggtggccaccatttgctccatgactttgttggttga

SEQ ID NO: 124 - protein, 570 aa, Sorghum bicolor
  misc_feature: Ceres ANNOT ID no.6039739
MSSAAGGGGYGAGGGAEHQHLLLGQAAGQLYHVPQHSRREKLRFPPDPADSPPPTAWPAPPPFYSYASSSTSSYSPHSPTPLAHAQLVAHALPAGAGAQIPSQNFALSLSSASSNPPPAPRRQLAAGVATGPYGPFTGYAAVLGRSRFLGPAQKLLEEICDVGGRPPHLDRRSDEGMLDMDAMDVVGDVDHDMDGGDRATAEAVAVSGAEQQWRKTRLISLMEDVCRRYKQYYQQLQSVISSFETVAGLSNAAPFASMALRTMSKHFKCLKEMIMSQLRNTSKVVANDGIGKDDMANFALMGGGAGLLRGNNVNAFGQPHNIWRPQRGLPERAVSVLRSWLFEHFLHPYPTDSDKQMLAKQTGLTRNQVSNWFINARVRLWKPMVEEIHNLEMRQVQKNTSVDKNQLGMQQIQHSTDSSGKLSDPSNSQRGQSSGMTRNLSSPASRHIQDELSQMPHDMPGQVSFAYNGLPTHHGLALSHPQQAEAVSAGIGLGGVAAGGGGVSLTLGLHQNNRTYIAEPLPAALPLNLPHRFGLEDVSDAYVMGSFGGGQDRHFTKGIGGHHLLHDFVG

SEQ ID NO: 125 - DNA, 1965 bp, Zea mays subsp. mays
  misc_feature: Ceres CLONE ID no.398671
atgtcctcggcggcgggcggtggatacggggctgcgggcggcgccgagcaccagcacctgttgctggggcaggcctcggggcagctctaccacgtgccgcagcacagccgccgcgagaagctgcggttcccgcccgacccggccgactcgcccccgcccactgcctggccggcgcctccacccttctactcctacgcgtcctcgtcgacgtcgtcctactcgccgcatagcccaacgctggcgcacacgcagctagtggcgcacgcgctacctgccggcgccggggcccaaatcccgagccagaactttgcgctctcgctctcttcggcgtcctcgaaccctccgcccgcgccgcggaggcagctcgccgcgggggtagccgctgggccgtacggcccgttcacgggctacgcggccgtcctcgggcggtccaggttcctgggccccgcgcagaagctactcgaggagatctgcgacgtgggcggacggcccccgcacctggaccggcgctccgacgacgagggtatgctcgacatggacgcggcggggggcgtggaccacgagatggacggcggtgactgcgctaccgctgaggcggtcgccgtctcgggcgccgagcagcagtggaggaagacaaggctcatctccctcatggatgacgtctgcaggcgatacaagcagtactaccagcagctgcaatccgtgatctcctcgttcgagactgttgcagggctgagcaacgcggcccccttcgctttcatggctcttcggacgatgtcgaagcacttcaagtgtctgaaggggatggtgatgagccagctacggaacacgagcaaggtcatcgccaaccatgggattattgccaaggacgatatggcgaacttcgcgctcatgggtggtggagcaggcctcctgaggggaaacagcgtgaacgcgttcagccagcctcacaacatttggaggcctcaaagagggcttcctgagcgtgcagtgtctgttcttcgctcatggctgtttgagcacttcttgcatccgtatccaactgatagtgacaagcagatgctggcgaaacagacagggttaacaaggaaccaggtgtccaattggttcatcaacgcaagggtgcgtctctggaagccgatggtggaagagatccacaacctggagatgcggcagctgcacaagaccacgtcggtggaccagaatcaactcggcatgcagcagcagaaccaccactcgtcggacggcagcgggaggccttcctccgacccttcgaactcccagcggggccaaagcagcggcatgacaagaaacctcagctctcgggcaccacgccacatccaggacgacgagctctcccagatgccccacgacatggcaggacaggtgagctttgcgtacagtgggctcccccccgcgcaccacggtctcgcgctgtcacaccaccaccacccccagcaagcgcaagctgaggcggttggtgttggcggcgtggccgccagcagcggcggcggcgtttccctcacgcttggccttcaccagaacaacaacaacaacagggcctatatcgccgagcccctcccggccgcgctcccgctcagcctcccgcaccggttcgggctggaggacgtgagcgacgcctacgtgatggggccgttcggcggccaggaccgccatttcagcaaggggatcggtggtgggcacaacttgctccatgactttgtcggttgatcaaattcgtccctggaagtgtttgtcggcatgtattgtattgttaggggtagtagggcagtggaagagagacgaccgtttggtccatctgcatccaagaacgctgtcgatatttgccaactcgaggtctatagagtggttgacgacataccgttgaagatgtgttccagttgtatgttgtagagttgaacgaaccctatctgttctaatcccagctggtgtggcgttaacacg

SEQ ID NO: 126 - protein, 642 aa, Vitis vinifera
  misc_feature: Public GI ID no.147770644
MADGFEPYHVPQQSRRDKLRVVAQNHSGCVEAAXNLHGCAGLLPLYDPSLLPSDLLTCASASAHEFQHHSHPLSGSAEACKANPGCVVKEEGVNLMGYVGGIMNASSSSSTSHHPYLDPQSSLPINPSSIQDMNHNPFFYAPQNLRDFDQSFNGGEMVVFKPEPLSLTHHESNTTGQGLSLSLSSHHTHQNNLPLELNLQRYGSAIFSDKVTGGYMVPGIVGGSGSTSNDVSRSSVPLGPFTGYASILKGSRFLKPAQQLLEEFCDVGCGLYAERVSADSSMMDPPMESLSGTGIVDDPLSCGDGGEHRRKKSRLISMLDEVYRRYKHYYQQMQAVVASFESVAGLGNAAPYADLALKAMSKHFRCLKNAITDQLQFTNKAHGQISHGKDESPRFGNTDRGLYGQRPMHSSGFLEHQPVWRPQRGLPERAVTVLRAWLFEHFLHPYPTDTDKLMLAKQTGLSRNQVSNWFINARVRLWKPMVEEIHTLETRQAQKSSQREERSADRQSDHLPSANSLVFENPSTSAQRVQDAPSKRTRNELSEVHVGSEEPMNLSYNNLSAHPHVGVGVSTAGGSSNVSLTLGLHQNNGIGLSESFPINAAQRFGLGLDANSEGYVIGGFEAQNRHFGRDVIGGQLLHDFVG

SEQ ID NO: 127 - protein, 612 aa, Oryza sativa subsp. indica
  misc_feature: Public GI ID no.125528380
MSSAAGGGGYGGGQGGGAEHHHHHHGHAGHLLLHHHPQHVAGAAVAAAAAAAGGQMYHVPQHSRREKLRFPPDAGDSPPPHGHGHGHAPQQQQQHGSWPPPPAFYSYASSSSSYSPHSPTLAQAQLVAHGLAPPLPQIPTQNFSLSLSSASSNPPPPQAQPRRQLGGLAQATGPFGPFTGYAAVLGRSRFLGPAEKLFEEICDVGGAASHVDRTISDEGLLDADPMDGVDHDVVDHDLGGADRAAADAGPISGAEQQWKKTKLISMMEEVCKRYRQYYQQVQAVMASFETVAGFSNAAPFAALALRAMAKHFKCLKSMILNQLRNTSNKVAVKDGLNKEIAVFGLAGGSSGGAGLQRANSASAFGQPHNIWRPQRGLPERAVSVLRAWLFEHFLHPYPTDGDKQMLAKQTGLTRNQVSNWFINARVRLWKPMVEEIHNLEMRQMHKHSVVDKGQHSVHHQAQHSSQCSGNPSVPSDSHPGQSSSITRNHNTAASQGFPDELSQMSQSIQGQVSFAYNGLTSQHNIASPHHQHQQVGGVGIGGSNGGVSLTLGLHQNNRVCIAEPLPAALPANLAHRFGLEEVSDAYVMSSFGGQDRHFGKEIGGHLLHDFVG

SEQ ID NO: 128 - protein, 600 aa, Oryza sativa subsp. indica
  misc_feature: Public GI ID no.125552568
MSSAAGGGGGYGGGGGEHQHQQQHHHLLLGQAAGQLYHVPQHSRREKLRFPPDHPAESPPPPPPGSWPLPPAFYSYASSSSSYSPHSPTLAHSQLVAHGMPPGAATSGGAQIPSQNFALSLSSASSNPPPTPRRQFGGGGGGAAGPYGPFTGYAAVLGRSRFLGPAQKLLEEICDVGGRPAQLDRGSDEGLLDVDAMEAAGSVDHEMDGSDRAVADAVTVSGAEQQWRKTRLISLMEDFKALLSSLLKLAGGDPQFIYNQKVCKRYRQYYQQLQAVVSSFETVAGLSNAAPFASMALRTMSKHFKYLKGIILNQLRNTGKGATKDGLGKEDTANFGLMGGGAGLLRGNNVNSFSQPHNIWRPQRGLPERAVSVLRAWLFEHFLHPYPTDSDKQMLAKQTGLTRNQVSNWFINARVRLWKPMVEEIHNLEMRQLKNPSLDKNQLSMQHTQHSSDSSGKPCDPSNSLQGQSSSMTRNHSISASRHIEDGLSQMPHDISGQVSFAYNGLAAHHSIAMAHHHQPDLIGTGGAANAGGVSLTLGLHQNNNRAYIAEPLPAALPLNLAHRFGLEDVSDAYVMSSFGGQDRHFTKEIGGHLLHDFVG

SEQ ID NO: 129 - protein, 580 aa, Oryza sativa subsp. japonica
  misc_feature: Public GI ID no.115464243
MSSAAGGGGGYGGGGGEHQHQQQQHHLLLGQAAGQLYHVPQHSRREKLRFPPDHPAESPPPPPPGSWPLPPAFYSYASSSSSYSPHSPTLAHAQLVAHGMPPGAATSGGAQIPSQNFALSLSSASSNPPPTPRRQFGGGGGGGGAAGPYGPFTGYAAVLGRSRFLGPAQKLLEEICDVGGRPAQLDRGSDEGLLDVDAMDAAGSVDHEMDGSDRAVADAVTVSGAEQQWRKTRLISLMEDVCKRYRQYYQQLQAVVSSFETVAGLSNAAPFASMALRTMSKHFKYLKGIILNQLRNTGKGATKDGLGKEDTTNFGLMGGGAGLLRGNNVNSFSQPHNIWRPQRGLPERAVSVLRAWLFEHFLHPYPTDSDKQMLAKQTGLTRNQVSNWFINARVRLWKPMVEEIHNLEMRQLQKNPSLDKNQLSMQHTQHSSDSSGKPCDPSNSLQGQSSSMTRNHSISASRHIEDGLSQMPHDISGQVSFAYNGLAAHHSIAMAHHHQPDLIGTGGAANAGGVSLTLGLHQNNNRAYIAEPLPAALPLNLAHRFGLEDVSDAYVMSSFGGQDRHFTKEIGGHLLHDFVG

SEQ ID NO: 130 - protein, 603 aa, Oryza sativa subsp. japonica
  misc_feature: Public GI ID no.125594476
MSSAAGGGGGYGGGGGEHQHQQQQHHLLLGQAAGQLYHVPQHSRREKLRFPPDHPAESRLPPPPGSWPLPPAFYSYASSSSSYSPHSPTLAHAQLVAHGMAPGAATSGGAQIPSQNFALSLSSASSNPPPTPRRQFGGGGGGGGAAGPYGPFTGYAAVLGRSRFLGPAQKLLEEICDVGGRPAQLDRGSDEGLLDVDAMDAAGSVDHEMDGSDRAVADAVTVSGAEQQWRKTRLISLMEDFKALLSSLLKLAGGDPQFIYNQKVCKRYRQYYQQLQAVVSSFETVAGLSNAAPFASMALRTMSKHFKYLKGIILNQLRNTGKGATKDGLGKEDTTNFGLMGGGAGLLRGNNVNSFSQPHNIWRPQRGLPERAVSVLRAWLFEHFLHPYPTDSDKQMLAKQTGLTRNQVSNWFINARVRLWKPMVEEIHNLEMRQLQKNPSLDKNQLSMQHTQHSSDSSGKPCDPSNSLQGQSSSMTRNHSISASRHIEDGLSQMPHDISGQVSFAYNGLAAHHSIAMAHHHQPDLIGTGGAANAGGVSLTLGLHQNNNRAYIAEPLPAALPLNLAHRFGLEDVSDAYVMSSFGGQDRHFTKEIGGHLLHDFVG

SEQ ID NO: 131 - DNA, 1726 bp, Glycine max
  misc_feature: Ceres CLONE ID no.475075
agtgacagagtgattggtgtgagtgcggcaattactggaacagcggttgagtgtgcgatcgagaaagcagagaaatggggttggaatcggtgggagatttagcgattcacgtgattctcagtaagttaggagcccaagacactgccagagtcgcgtgtgtgagcaaaaggttctgttcttccgcttccgacgacactctctggatcaatcactgcttccacgaactcgctctgacacaacccctcgatcatctcggaaaccctctctcttccttcaaggaatgctatcaagcatggcgaggagcttttgttatgtatccttggtctcttgttaagcgtgtaaaaaggtgctgggataaaataaaaacctggttgaccaataattttcctgaagctgaggccactctttgtaaaggtgctactgaagctgacattcaggagttggagaatgtattaaaggtgaaattgccacttccttcaaggatcctttatcgttttcacaatgggcaagaaattgcaaaagcagatccagaaactactacatatggcagttctttgggtctaattggtggctactccttctatagtcatttggtgaatgtttatctattacctatacgtcagataatcctagaaacaaagcaaactaggcgtcacttgagctttttaagaagatcaaagtatgttctcgtggctgcttcatccacatacagtagaaagttatttttcctcaactgtaccaatggtcaactatatgttgggaccagggatctacttactgaaggagacataatcccttgtgtacctcatgatctgattaatttacatcaggaattgaatatttcagagcaacaagatgccatgctactgtggttagaagaacatggtcgccgtttagaacatggctttatcaaacttcatgacaaaggaaatggcaaaagcattaatcttttcccagaagaaccccctctttgttcaatggctgttactaatggtgtgaaggttcgtgcttctgcattggttatccctgagttgattgatcttcaagatgaccttgaaaagtacttatttgcttattcaatccgcttgtcccttgaacctcaagggtgcactattaatggaatgtccttcagctcctgccagctccattggaggcactggatcatccgagctaatgatattgttatatctgatgtcaatggagaagctgttataggacagtatccacttttgcgtccaggtgctcaagaatttgtctatcagagtcgcatgcatctaccaacaccatcaggttccattgaaggttcttttacatttatacccggcagattggcagacccaaaaggagatccttttctagctacagtggctcgtttcccgctccagcttccagactatattttctgattttgatgctagatgggattgaagcatctctaatggcaaatgcagctctcagtatattgtttgggtgcattatgtcattttcgtgagagttgctaggtcgttgagttttttaagttggacctgcacaatttccctatctattatatatcaatatctatctatctgtatctagtgtggtgtagtaataattcgattacttgcgttcttgacttgtgtattaacttcctatccttcattcattgtacaaatctatatgcatgcagcaattcaatgatacatatttagtattgtcgtctataacatgggaacgtgtttgtgtt

SEQ ID NO: 132 - DNA, 1398 bp, Populus balsamifera subsp. trichocarpa
  misc_feature: Ceres ANNOT ID no.1455953
atggggctggaatcagtgggagatctagctctgaacataatcttaacaaaacttggtccaaaagagacagtacaagtactatgtgtcagcaaaaagttcaaggatttagcttcagaggaatctctctggtcattattttgccgtcaagatcttgatctttctgctcctcttgaccatcatggaaatcatctgccttcttttaaggcaacttataagttatggagagaagcctttcatatgtatccttggccccttgtaaagcgagttaaaagttgttgggacagactcacgagctggttgaccgcgaactttcctgaagttaaggctaccctaggaaagggtgcatcagaaggtgagattcaaaagttggaaagaattttgaaagttaagttgcctcttcccacaagacttctctaccgctttcatgatggtcaacatttctcagacaaaaatctgtcaggtggcatggctggttgtccattgggcctgataggtggctactgtttttataatcactcggttaatgtctacttattatcactacatgaggtaatctctaaaacgcaggaaatagtgcggcacctgaacttacccgatacatccgagtatattgttgtggctgcttcatcctcatacgttggaaagtttttcttcctgaactgttctgatggccaactctatgttgggacccagaattttccaacagatgcagaaatgatgccatgtgtacctcaggcattgattagtccagtccgtgatttcaacagtgaccaacaacaggatgctatgttgttatggttagaagaacatggccgtcgcttgcacaatggcatgatcaaaattctcggcaaaggaaatattaaaagcatctctcagtttccagaagaatctcctctctgttcaactgctgtaaccagtggtgtaaaggttcgtgcttctgctgtttttgtgccagaggctgctgatctggaagatatttctacaaaatacgtgttcgcttattcaatccgcatgtcccttctaccagaaggatgcatcatcaacggaatgcacttcagctcttgccaactgcacctgaggcactgggttatcagtgctaatgatactgctgtatctaatgtcaatgcagaggctgtgataggcaagggtcctcctgtgtggccttctagatgcaacaattgggagcttctaaaagtgcctactggtgacaaattcccactcttgtttccaggcgagaaagaatttgtttatgagagttgtacacctctgccaacttctactggctctgttgaaggttctttcacatttgtccctggcagattggcagatccaaaaggaattccatttgaagttgaagtcggtcggtttccgctccaactgccagactacattttctga

SEQ ID NO: 133 - protein, 465 aa, Populus balsamifera subsp. trichocarpa
  misc_feature: Ceres ANNOT ID no.1455953
MGLESVGDLALNIILTKLGPKETVQVLCVSKKFKDLASEESLWSLFCRQDLDLSAPLDHHGNHLPSFKATYKLWREAFHMYPWPLVKRVKSCWDRLTSWLTANFPEVKATLGKGASEGEIQKLERILKVKLPLPTRLLYRFHDGQHFSDKNLSGGMAGCPLGLIGGYCFYNHSVNVYLLSLHEVISKTQEIVRHLNLPDTSEYIVVAASSSYVGKFFFLNCSDGQLYVGTQNFPTDAEMMPCVPQALISPVRDFNSDQQQDAMLLWLEEHGRRLHNGMIKILGKGNIKSISQFPEESPLCSTAVTSGVKVRASAVFVPEAADLEDISTKYVFAYSIRMSLLPEGCIINGMHFSSCQLHLRHWVISANDTAVSNVNAEAVIGKGPPVWPSRCNNWELLKVPTGDKFPLLFPGEKEFVYESCTPLPTSTGSVEGSFTFVPGRLADPKGIPFEVEVGRFPLQLPDYIF

SEQ ID NO: 134 - DNA, 1428 bp, Populus balsamifera subsp. trichocarpa
  misc_feature: Ceres ANNOT ID no.1541547
atggcgtccagctttactttcatcgagttgaaagctgaaacttttcaacatgtgtgcatattttcaaagttctattgggcaacttataagttatggagagaagctttttgcatgtatccatggtcccttgtaaagcgagttaaaagttgttgggacagactcaagagctggttgaccacaaactttcctgaagttaaagctactcttggaaggggtgcatcagaaggtgagattcaagagcttgaaagaattttgaaagttaagctgcctcttcccacgaggcttctctaccgctttcatgatggtcaaaacttaacaggtgaaaatttgaacactgatgcggctgcctgtctattgggcctgataggtggctactgtttttatgatcacttggttaatgtctacttattacccctacatgaggtgatcttggaaacacgggagatagtacgccacctggacttacccaatggatcacagtttattgttgtggcagcctcatcctcaaacattggaaagtttttcttcctgaactgttctgatggccaactttatgttgggactcagaatcttctgacaattggagaaatgatcccatgtgtacctcagacattaattagtccagtccatgatttcaacatagaccaacaacaggatgctatgttgttatggctagaagaacatggccatcgcttgcacaatggcatgatcaaacttcgggatgaaggaaatatcaaaagtatctctctttttccagaagaatctcctctctgctcaactgctgtaaccaatggtgtaaaggtccgcgcatctgctatttttgtgccagaggctgttgatctctctagaaaatacttgtttgcttattcaatccgcatgtcccttccaccagaaggatgcatcatcaatggaatgcgcttcagctcttgccaactgcatttgaggcactgggttatcagcgccgatgatactgtcgcatctaatgtcaatgcagaggctgtgataggcaagttcccactcttgcttccaggtgagaaagaatttgtttatgagagttgcacacctctgcgatctcctactggctctgttgaaggttctttcacatttgtccctggcaggaagaaaaagctagctccatgtgagcaactgtggcgcaataacaagaatactatagcaatcactgcaaaaataatcttaacaaaaagactggtaatcactgcaaaaataatcgtactcactccggctcgtacaatgatgcctgttgcctcatcggaaggatctctgacagtaaacccggttgccaaggtacgtgagaagttgccgggtgttctagcactacatgccttaatgataggtttatatctaagagatcctttcgacgaggaagcaacaggcaaagagcatcactatgggggtcacattgattga

SEQ ID NO: 135 - protein, 475 aa, Populus balsamifera subsp. trichocarpa
  misc_feature: Ceres ANNOT ID no.1541547
MASSFTFIELKAETFQHVCIFSKFYWATYKLWREAFCMYPWSLVKRVKSCWDRLKSWLTTNFPEVKATLGRGASEGEIQELERILKVKLPLPTRLLYRFHDGQNLTGENLNTDAAACLLGLIGGYCFYDHLVNVYLLPLHEVILETREIVRHLDLPNGSQFIVVAASSSNIGKFFFLNCSDGQLYVGTQNLLTIGEMIPCVPQTLISPVHDFNIDQQQDAMLLWLEEHGHRLHNGMIKLRDEGNIKSISLFPEESPLCSTAVTNGVKVRASAIFVPEAVDLSRKYLFAYSIRMSLPPEGCIINGMRFSSCQLHLRHWVISADDTVASNVNAEAVIGKFPLLLPGEKEFVYESCTPLRSPTGSVEGSFTFVPGRKKKLAPCEQLWRNNKNTIAITAKIILTKRLVITAKIIVLTPARTMMPVASSEGSLTVNPVAKVREKLPGVLALHALMIGLYLRDPFDEEATGKEHHYGGHID

SEQ ID NO: 136 - DNA, 1623 bp, Populus balsamifera subsp. trichocarpa
  misc_feature: Ceres ANNOT ID no.1488131
atggcgtccagctttactttcatcgagttgaaagctgaaacttttcaacatgtgtgcatattttcaaagttctattgggcaacttataagttatggagagaagctttttgcatgtatccatggtcccttgtaaagcgagttaaaagttgttgggacagactcaagagctggttgaccacaaactttcctgaagttaaagctactcttggaaggggtgcatcagaaggtgagattcaagagcttgaaagaattttgaaagttaagctgcctcttcccacgaggcttctctaccgctttcatgatggtcaaaacttaacaggtgaaaatttgaacactgatgcggctgcctgtctattgggcctgataggtggctactgtttttatgatcacttggttaatgtctacttattacccctacatgaggtgatcttggaaacacgggagatagtacgccacctggacttacccaatggatcacagtttattgttgtggcagcctcatcctcaaacattggaaagtttttcttcctgaactgttctgatggccaactttatgttgggactcagaatcttctgacaattggagaaatgatcccatgtgtacctcagacattaattagtccagtccatgatttcaacatagaccaacaacaggatgctatgttgttatggctagaagaacatggccatcgcttgcacaatggcatgatcaaacttcgggatgaaggaaatatcaaaagtatctctctttttccagaagaatctcctctctgctcaactgctgtaaccaatggtgtaaaggtccgcgcatctgctatttttgtgccagaggctgttgatctctctagaaaatacttgtttgcttattcaatccgcatgtcccttccaccagaaggatgcatcatcaatggaatgcgcttcagctcttgccaactgcatttgaggcactgggttatcagcgccgatgatactgtcgcatctaatgtcaatgcagaggctgtgataggcaagttcccactcttgcttccaggtgagaaagaatttgtttatgagagttgcacacctctgcgatctcctactggctctgttgaaggttctttcacatttgtccctggcaggaagaaaaagctagctccatgtgagcaactgtggcgcaataacaagaatactatagcaatcactgcaaaaataatcttaacaaaaagactggtaatcactgcaaaaataatcgtactcactccggctcgtacaatgatgcctgttgcctcatcggaaggatctctgacagtaaacccggttgccaaggtacgtgagaagttgccgggtgttctagcactacatgccttaatgataggtttatatctaagagatcctttcgacgaggaagcaacaggcaaagagcatcactatgggggtcacattgagaccccccttggtagctcgggccttctgttgcttctgtctgccaatggagttgacttcttcttccttgcgggttccaccatggtagctcgggccttctgtttcttctatcagaagacagggagacagtccttggttttgaagtgttgggtggtcatggtaccagtaggatctcttaacatgaacacaatgagatggtga

SEQ ID NO: 137 - protein, 540 aa, Populus balsamifera subsp. trichocarpa
  misc_feature: Ceres ANNOT ID no.1488131
MASSFTFIELKAETFQHVCIFSKFYWATYKLWREAFCMYPWSLVKRVKSCWDRLKSWLTTNFPEVKATLGRGASEGEIQELERILKVKLPLPTRLLYRFHDGQNLTGENLNTDAAACLLGLIGGYCFYDHLVNVYLLPLHEVILETREIVRHLDLPNGSQFIVVAASSSNIGKFFFLNCSDGQLYVGTQNLLTIGEMIPCVPQTLISPVHDFNIDQQQDAMLLWLEEHGHRLHNGMIKLRDEGNIKSISLFPEESPLCSTAVTNGVKVRASAIFVPEAVDLSRKYLFAYSIRMSLPPEGCIINGMRFSSCQLHLRHWVISADDTVASNVNAEAVIGKFPLLLPGEKEFVYESCTPLRSPTGSVEGSFTFVPGRKKKLAPCEQLWRNNKNTIAITAKIILTKRLVITAKIIVLTPARTMMPVASSEGSLTVNPVAKVREKLPGVLALHALMIGLYLRDPFDEEATGKEHHYGGHIETPLGSSGLLLLLSANGVDFFFLAGSTMVARAFCFFYQKTGRQSLVLKCWVVMVPVGSLNMNTMRW

SEQ ID NO: 138 - DNA, 1350 bp, Sorghum bicolor
  misc_feature: Ceres ANNOT ID no.6098347
atggcgtccccgccgccgccagaggccgcaccggctgaggcggggctcgagagtatggagggcctcgtcctggacacggtcatctcaagggccggcgcgcgccccgcagcggcgctcgcctgcgccagcacgcgcctccgcaccgccgtcgccgacgactccctctggcgcagattctgcgctgaggacctggggctcgacgcgcccgtcgatccggagggtcagcctctcccctcgttccaggttgcatataaggtttggttggagtcttttggcatgtaccctctacctatggtaaagagagtgaaacaattttggacttcaatgaaaacttggttgtctgaaaactttcccgaggcatacaaaacgttgtgcaaaggtgtttctgaagctcaattaaagtcggcagaggatgatcttggtttcaagctccctatgcccacaaagctgttgtatcgcttttgcaatgctcaactgccttttagtgaagaccatgatgcaaacaaaagcatttccactcatggattgattgggggctatgcattttatgatcattgggtgaatgtgcatttgtcacctcttgagcaaatagttgaagagacaaaggacttctatcgggagttccctgatgtctttaatgggcgcaacttcattgtagtggcgacttcatggtttcgtccgaaaacgtttcttctagattgctcaaatggtgaactttatgttggtacatacaacttacctataggaggaatgcttccctgtgtccctaaagcattgataaagccagcagagaatgatctagcccaagatggactacttctgtggttagaagagcatcttaggcgtttacagaatggcatgatcaagacccgtatgctgatgacatcgagatatatcagcttatatccagaagcacctccatcatgcagttcagctgttacaaatggcattaaggtacgctcgtctgctgtctttgtgccagaacatcctggggggcctggggaaaaatttatgttcacttactcgattcgcatgtcagttccagaggcatgcatgctaggtggtgtgtactattcttgctgccagctttcctcccgccactggaccatcagatcgtgtgacagggttgtttctgatgtgagcggaggaggtgttattggacagtatcctgtactgttacctggtgaggatgagtttgtctacgagagttgcacgccactgcccaaagtacctggatctgtggagggttctttctcgtttgtgcctggcaagttgatccgacctgaagggaagcctttcgaagtcatggtggcgccattccctcttgaggtgccagaatacatcttctga

SEQ ID NO: 139 - protein, 449 aa, Sorghum bicolor
  misc_feature: Ceres ANNOT ID no.6098347
MASPPPPEAAPAEAGLESMEGLVLDTVISRAGARPAAALACASTRLRTAVADDSLWRRFCAEDLGLDAPVDPEGQPLPSFQVAYKVWLESFGMYPLPMVKRVKQFWTSMKTWLSENFPEAYKTLCKGVSEAQLKSAEDDLGFKLPMPTKLLYRFCNAQLPFSEDHDANKSISTHGLIGGYAFYDHWVNVHLSPLEQIVEETKDFYREFPDVFNGRNFIVVATSWFRPKTFLLDCSNGELYVGTYNLPIGGMLPCVPKALIKPAENDLAQDGLLLWLEEHLRRLQNGMIKTRMLMTSRYISLYPEAPPSCSSAVTNGIKVRSSAVFVPEHPGGPGEKFMFTYSIRMSVPEACMLGGVYYSCCQLSSRHWTIRSCDRVVSDVSGGGVIGQYPVLLPGEDEFVYESCTPLPKVPGSVEGSFSFVPGKLIRPEGKPFEVMVAPFPLEVPEYIF

SEQ ID NO: 140 - protein, 515 aa, Oryza sativa subsp. indica
  misc_feature: Public GI ID no.125534006
MAAPPQPQPEPEPAAGGAGLEALEGLALDTVIAKAGARQAAALACASTRLRDAAGDDALWRRFCADDLALHAPLAPDGRALPSFKNSIFEQCDGISLCEPGCGDSSPFHHASLDTDVAWLLLPPFWPPESTVIRQIGIEWEDKDAYKVWLESFGMYPLPLVRRVKIFWSSLKSWLSENFPEAHKTLNKGVSEAQIQSAEDDLGFKLPLPTKLLYRFCNGQLPLSEHHHENMRMAHLGIIGGYVFYDHLINVHLSPLEQIVEETKEFYREFYDQGVFNMTNLIIVATSWYRPKTFFLNCSDDQLYVGTINLQDGEMLPCVPKSLIRPTNTDMPQDGLLLWLEEHLRRLQNGMIKIRMLKTSRYISLFPEASPSCTSAMTNGVKVRASAVFAPEHPESRRPGAKCLYAYSIRLSVPEACMLGGVYYSSCQLYSRHWIIRWRDRVVSDVNGEGVIGKYPLLTTGQEEFVYESCTPLPDSPGSVEGSFTFVPGKLSRPEGKPFEVTVAPFPLEIPEYIF

SEQ ID NO: 141 - protein, 442 aa, Oryza sativa subsp. indica
  misc_feature: Public GI ID no.125534002
MASPAKAQRRPEGASVLETLPALPLAIIIAKAGPRCAAALACASSTLRAAASGEALWRHFCSDDFALDAPLSPGDLPLPSFKDAYKAWFQSFGMYPLPLVKRVKIFWSSFRAWLCEYFPEGLRTLGEGVSEAEIAVAECNLGLVLPMPTKLLYRFCNGQLHIGRGEEVSYGVMGGYDYVHQRYTVRLLPLAHHAVQKNSNYIVVATSCFGEKIFLLDCASGRLYVGTKYWNEEREIMACVPKATIRLAVDDDHGMPQDGFLLWLEEHLSRLQDGLIKVQSCKFPMLARHISLYPVQLPYCSSASMHGIKVRASAVFAPENSAFADYRCRYSYYFSIRLSLPEAFVVDGKWYSSFQLQSCHYTIQIGDEVLPYTCNYGGHGKCPLLRCGEELFVYGCSISAALEPGSVMGNLTLVPWRCGQPRGSPFIADIAPFPLHPPDYIF

SEQ ID NO: 142 - protein, 495 aa, Oryza sativa subsp. japonica
  misc_feature: Public GI ID no.115485029
MGVDSHPNNINDGRLLLSPPPLLPILISDTAAAISPISMAAPPQPQPEPEPAAGGAGLEALEGLALDTVIAKAGARQAAALACASTRLRDAAGDDALWRRFCADDLALHAPLAPDGRALPSFKDAYKVWLESFGMYPLPLVRRVKIFWSSLKSWLSENFPEAHKTLNKGVSEAQIQSAEDDLGFKLPLPTKLLYRFCNGQLPLSEHHHENMRMAHLGIIGGYVFYDHLINVHLSPLEQIVEETKEFYREFYDQGVFNMTNLIIVATSWYRPKTFFLNCSDDQLYVGTINLQDGEMLPCVPKSLIRPTNTDMPQDGLLLWLEEHLRRLQNGMIKIRMLKTSRYISLFPEASPSCTSAMTNGVKVRASAVFAPEHPESRRPGAKCLYAYSIRLSVPEACMLGGVYYSSCQLYSRHWIIRWRDRVVSDVNGEGVIGKYPLLTTGQEEFVYESCTPLPDSPGSVEGSFTFVPGKLSRPEGKPFEVTVAAFPLEIPEYIF

SEQ ID NO: 143 - protein, 417 aa, Oryza sativa subsp. japonica
  misc_feature: Public GI ID no.125576804
MSGCGDSSPFHHASLDTDVAWLLLPPFWPPESTVIRQIGIEWEDKDAYKVWLESFGMYPLPLVRRVKIFWSSLKSWLSENFPEAHKTLNKGVSEAQIQSAEDDLGFKLPLPTKLLYRFCNGQLPLSEHHHENMRMAHLGIIGGYVFYDHLINVHLSPLEQIVEETKEFYREFYDQGVFNMTNLIIVATSWYRPKTFFLNCSDDQLYVGTINLQDGEMLPCVPKSLIRPTNTDMPQDGLLLWLEEHLRRLQNGMIKIRMLKTSRYISLFPEASPSCTSAMTNGVKVRASAVFAPEHPESRRPGAKCLYAYSIRLSVPEACMLGGVYYSSCQLYSRHWIIRWRDRVVSDVNGEGVIGKYPLLTTGQEEFVYESCTPLPDSPGSVEGSFTFVPGKLSRPEGKPFEVTVAAFPLEIPEYIF

SEQ ID NO: 144 - protein, 421 aa, Oryza sativa subsp. japonica
  misc_feature: Public GI ID no.108864217
MAAPPQPQPEPEPAAGGAGLEALEGLALDTVIAKAGARQAAALACASTRLRDAAGDDALWRRFCADDLALHAPLAPDGRALPSFKDAYKVWLESFGMYPLPLVRRVKIFWSSLKSWLSENFPEAHKTLNKGVSEAQIQSAEDDLGFKLPLPTKLLYRFCNGQLPLSEHHHENMRMAHLGIIGGYVFYDHLINVHLSPLEQIVEETKEFYREFYDQGVFNMTNLIIVATSWYRPKTFFLNCSDDQLYVGTINLQDGEMLPCVPKSLIRPTNTDMPQDGLLLWLEEHLRRLQNGMIKIRMLKTSRYISLFPEASPSCTSAMTNGVKVRASAVFAPEHPESRRPGAKCLYAYSIRLSVPEACMLGGVYYSSCQLYSRHWIIRWRDRVVSDVNGEGVIGKVCGKQEEHSINYVFLHAHIHFKRKV

SEQ ID NO: 145 - protein, 442 aa, Oryza sativa subsp. japonica
  misc_feature: Public GI ID no.115485023
MASPAKAQRRPEGASVLETLPALPLAIIIAKAGPRCAAALACASSTLRAAASGEALWRHFCSDDFALDAPLAPGDLPLPSFKDAYKAWFQSFGMYPLPLVKRVKIFWSSFRAWLCEYFPEGLRTLGEGVSEAEIAVAECNLGLVLPMPTKLLYRFCNGQLHIGRGEEVSYGVMGGYDYVHQRYTVRLLPLAHHAVQKNSNYIVVATSCFGEKIFLLDCASGRLYVGTKYWNEEREIMACVPKATIRLAVDDDHGMPQDGFLLWLEEHLSRLQDGLIKVQSCKFPMLARHISLYPVQLPYCSSASMHGIKVRASAVFAPENSAFADYRCRYSYYFSIRLSLPEAFVVDGKWYSSFQLQSCHYTIQIGDEVLPYTCNYGGHGKCPLLRCGEELFVYGCSISAALEPGSVMGNLTLVPWRCGQPRGSPFIADIAPFPLHPPDYIF

SEQ ID NO: 146 - protein, 417 aa, Oryza sativa subsp. japonica
  misc_feature: Public GI ID no.108864214
MASPAKAQRRPEGASVLETLPALPLAIIIAKAGPRCAAALACASSTLRAAASGEALWRHFCSDDFALDAPLAPGDLPLPSFKDAYKAWFQSFGMYPLPLVKRVKIFWSSFRAWLCEYFPEGLRTLGEGVSEAEIAVAECNLGLVLPMPTKLLYRFCNGQLHIGRGEEVSYGVMGGYDYVHQRYTVRLLPLAHHAVQKNSNYIVVATSCFGEKIFLLDCASGRLYVGTKYWNEEREIMACVPKATIRLAVDDDHGMPQDGFLLWLEEHLSRLQDGLIKVQSCKFPMLARHISLYPVQLPYCSSASMHGIKVRASAVFAPENSAFADYRCRYSYYFSIRLSLPEAFVVDGKWYSSFQLQSCHYTIQIGDEVLPYTCNYGGHGKCPLLRCGEELFVYGCSISAALEPGSVMGNLTLVPWR

SEQ ID NO: 147 - protein, 471 aa, Solanum demissum
  misc_feature: Public GI ID no.47824984
MAMVKLEATKSTSSNLLDPSFSSYMINGTEETIVLNLESSRDLSKKVDDGEIDIFSAEKYFNEGVDEENNVTQNKHKIHDDQPVADIVSLQQKIRPLTPSIHSESSWNSRSALLQKVSINHQYQHQHHQLPRPTKTNNKSYGKKFLARFGCNCYCKDKNSVEIDDQLREKSFKSKSKQNTIKTSTIGANHQNLHFKKIDELGVLGLKSDERFAVPVFDPKVGNPNPGVKIQLHKEEEEESRKSLEVFGFPITEKERSKMSLEKNIGMLTWDAIVPKAEEIDIINIGASSNGTYEEDYAESDASSDLFEIESFPSNNTANPSLVRQGSDSMSCYAPSEVSIDWSVVTASAADFSIMSDIEEVKIPSIRTTSNSNRSVSQNGRDKAKRRSGILLGCNSHKAVGVVGDAYKVSEKSSIEMHQRNFKTYEPIIPMTRFHAESRVNRFDGGNRKHEFTTRSFATTYSGRPADFLYI

SEQ ID NO: 148 - DNA, 1905 bp, Zea mays
  misc_feature: Ceres CLONE ID no.1580361
ccatggggatctttttagttacgcggctcgtcttctcgccttcttttttctctagtgctactgccactactgatccgacaggaaaaccgcagagaacaacgtgttcagagttctaactcgtgctgcttgctgtctgtaggcgtgctgaaatcactacaaactttacaagattcccctacaatggcgagcgatccgtcgacgttctcgccaatcggggtggacgccatgggcggcggctacttcatggccggcagcagcagctacggaggcggcggcattatgagcgccgaggtgccgcacttccaccaccccggcgtgcttcttgaccagggcggcttcgggttcggcggcctcggcaacgcggcggtcgtcggcggcgccgcgacggcggcggatcttggcgcgcactacgcggcgaacaacatcgtgctggcctccttcgccagccagctcctcgctaacgcccccgcgccgccgcgggatgatcgtgccggcgggaggacgccgccggacgagatggacgaagagctgtacggcgtcgccggctgcgacagccgcgtcgcggcgagcttgcggtgccccagccaatccggggccatggcggtctggtcgtcgccatcgtcgtccaagaagccgtacggcatctggaccagcgccggcggaccagctcatgaaccataccacctcgccgcggccgggctctccgacgcgggaggcctccgctacccgctcgccgcctgcagtggcggcaacgcgtccgcggcggcggcgagcgagctgtcgctgactctgtgctccaactccatcgcttcctcggacagcgcgctgaacgcgacggagcagtgctcctcgggagcgagccggtccgccctgaccgagctgccgcgggcgcggtcccggatggcgctgcacttcgccgcggtggtggcgcgctcgcggtacgcggccgtggtccaggatttgctcaacgacgtcgtcggccacatgctggacggcgtcgccgacgtgaccgacgactcgtgcagcggcatcggctcggttggcgcgccgtccgcggtgagctccaacaggttcatggcctcgaccgaagacgccggcgcgcgctggggccaggcccagagggtccggagcaacctcctcaagacgcttcagctgatggacgagaagtacaaccagtgcttggacgagatccagagcacgacggccaggttcaacaccctgatgcactcggccccgggcggcggcggcatctgcgcgccgttcgcgcaccgcgccgtgtcggcgatgtaccgcgggctgaggcggcggctcgccggcgagatcatggctgcggcgagcagggccagctgctggggcgagtcgtcgtcgtcggtgacggtggccgcgggcggcgacgtggagcggagctgggagtcggccttcatacagaagcactggtcggcgcagcagctgcggcgcaccgagcagcagtgctggcggccccagcgcggcctgccggagaagtccgtcgccgtgctcaaggcgtggatgttcgagaacttcctgcacccgtatcccaaggaccatgagaaggacgtgctggcggcaaggagcggcctcaccaggaaccaggtgtcgaattggttcataaacgcgcgggttcgtctatggaagccgatgatcgaggagatgtaccaggacctcaagaggagctcgggtgcaggaggaggagggcacggcccggcgatggagccacagcagcacttgagcaagcgtcgaatttgcgagctggaagatggtggccagtgaaccatccagctccatagctgcctgccgtggatttaccattggcccttgtagtttccctgtgctatgtttatgtacatatcattgccaacgccagcatttaacatctgcgcatctttt

SEQ ID NO: 149 - protein, 469 aa, Arabidopsis thaliana
  misc_feature: Public GI ID no.15215913
MDNNNNNNTFSSLDNVMTNQNPLLMDFIPSREDSTMLPWNTIRSDPLQMGGFDIFNSMLTNKYLSSSPRSIDVQDNRNVEFMAPPPHPPPLHPLDHLRHYDDSSNNMWGFEANSEFQAFSGVVGPSEPMMSTFGEEDFPFLISNKRNNELSLSLASDVSDECSEISLCAATRLASEQASCSSKDISNNVVTQGFSQLIFGSKYLHSVQEILSHFAAYSLDYSSRGTESGAASSAFTSRFENITEFLDGDSNNSEAGFGSTFQRRALEAKKTHLLDLLQMVDDRYSHCVDEIHTVISAFHAATELDPQLHTRFALQTVSFLYKNLRERICKKIISMGSVLERGKDKTQETSMFHQHCLLQQLKRKNHQIWRPQRGLPEKSVSVLRNWMFQNFLHPYPKDSEKHLLAIRSGLTRSQVSNWFINARVRLWKPMIEEMYAEMNKRKLNNSHIQPNGPTLRMPKSVMMSQAMHK

SEQ ID NO: 150 - DNA, 1957 bp, Triticum aestivum
  misc_feature: Ceres CLONE ID no.845859
ctttaaaaattagtagtgtgcaagatttggcctttggcgactacggagctaggcctcaagatgtctagcaatccatcctatcagcagcagctggggctggatgccatgaccacttgtttcttcggcggcagcggcatgattggttcttccgaagcgccattcttctacccaggcatgccgcacgatgcgggcttcggctccggcggcgcggaggtggccgcgcatttcatggccagcagcgccgtgatggtcacttcgccggcgaaccagctcgtgtggtccgccgctccttcgcgggacagccaccaagcgagcatgtccacggacgagatgaacgacgacgcctacgccgtcgccggcgagagctgcagcaccgtgcactccatgctcccttccgcctccggctcggcggacttcttccaatacgggccggcggaggtcaccatcgcccagccgtcaaagatggccaagctcattacgggagagccgcactgcggctggccctacgacggaccgagcgctgcctcgacccaccagccgtattacctgacggcgttctccggcggcggcgaccttccggacgcggtcgccggcgcggcgagcggcctgtcgctcaggctaggtgcccagtcttcctcggtcaccatggcgagcatgccggagcagtcctcggaggtgagctgctccggcctgacccatgtgaacagcgaaggctttggctaccagcagcctcaggccgtcagggctcacgccggcgccggccagttccatctgcctccttacggcgaggtcggtgccggcgactacgagctgcggcacgtgtacccacagatgtactcgagggctccgcacttctcgcaggtgctgccgcggtcgggatacgcgcacattgcccaggagctgctgaatgggttcgccggctgcctgctgaaggacgtggccgagatgactgacgattcagtcagcgacattggcagcgaggcgagcctcctgctctcctcgagctgctcggcgaggacgccgtcgtcggtgagctccaaccagctgatgctgccctccgacgagcactcagccgacggcgggaggtggatggaggctcagagggtgaggaacgatctcctgaaactgctgcagctgatggatcaaaggtgcaaccggtgcttcgacgacatccagacgacggcgtccaagttcagcagcatggtggcgcatccgggcggcggaggcggcgccatcgcgccgccgccgttcgcgcagcgcgcgctctccgcggtgtaccggaggctgaggaagcggatcacgggcctgatcgtggcggtggcgcagaggtccggcgggcacggggagccgtcgtcgctggcggacaaggagcggagctgggagtcgtccttcatccagaagcactgggcgctgcagcagctccggcgcggcgaccagcagtcgtggcggccccagcgcggcctgccggagaagtccgtcgccgtgctcaaagcctggatgttcgagaacttcctccgcccgtacccgaaagaccacgagaaggacatgctggcggcgaggagcggactgagcaggagccaggtctccaactggttcatcaacgcgcgcgtccggctgtggaagccgatgatcgaggagatgtacgaggagctcaagaggagctcggggcgcggcgacgccgagcatcagagcagcaaagacgtcgtcggctaggaaggggatggccgaggttggaactgcacggagccctagccgttgtgctttcttcagagcttccgtgcaagcattgctggccggccactgatgggtcctgctgtttctctgcccattgctcatcatctcatccctatctatgtagtaacctgtagttctcttgcgtgtacatatggctgccacccggtgcctgcgagttgcgattaaacgaaaaccactgtcgcatgatcgttgattgctcatg

SEQ ID NO: 151 - protein, 550 aa, Triticum aestivum
  misc_feature: Ceres CLONE ID no.845859
MSSNPSYQQQLGLDAMTTCFFGGSGMIGSSEAPFFYPGMPHDAGFGSGGAEVAAHFMASSAVMVTSPANQLVWSAAPSRDSHQASMSTDEMNDDAYAVAGESCSTVHSMLPSASGSADFFQYGPAEVTIAQPSKMAKLITGEPHCGWPYDGPSAASTHQPYYLTAFSGGGDLPDAVAGAASGLSLRLGAQSSSVTMASMPEQSSEVSCSGLTHVNSEGFGYQQPQAVRAHAGAGQFHLPPYGEVGAGDYELRHVYPQMYSRAPHFSQVLPRSGYAHIAQELLNGFAGCLLKDVAEMTDDSVSDIGSEASLLLSSSCSARTPSSVSSNQLMLPSDEHSADGGRWMEAQRVRNDLLKLLQLMDQRCNRCFDDIQTTASKFSSMVAHPGGGGGAIAPPPFAQRALSAVYRRLRKRITGLIVAVAQRSGGHGEPSSLADKERSWESSFIQKHWALQQLRRGDQQSWRPQRGLPEKSVAVLKAWMFENFLRPYPKDHEKDMLAARSGLSRSQVSNWFINARVRLWKPMIEEMYEELKRSSGRGDAEHQSSKDVVG

SEQ ID NO: 152 - DNA, 1929 bp, Zea mays
  misc_feature: Ceres CLONE ID no.354689
aacacaggcaagccattcgttctccctgctgccgttttattaggaaactctcccctttccgttttagcttagcgtccagctgcaaacttctcccccagcttctcttgcatctctctttgtatttcactgtgctggcctgctttgttccttcgttctggttactattggttgctgtttccaagtgacgtaccagagtggtggttggcatttcagtatgatttgataagcatctgggactgggagtcgacgccacgatgcgcagctgcttcgccagcggcgacggcagtgttatcagcagcgccgacgcgcccttcttccacgtcccggagtacacactgcacggcggcggcggctttgggtttggcgaacccgcagcggacgtcgccgcctcgagcttcttggcggatggttcggtgctgctcgccggccagctgctccgtgccaccgcactgcagagcgtgtcgccggaggagaggcacggtgcctactacggcgtcacgggcgggtcgtcgtcgtacggcccgtcgccctgggatgtcaccgttgctcacgcgcccagcaggatgaccaagcagcctgtcgctggagagccggaaggcggctggatccacgagtcttcgtactactgcccggcgacgtggttctctggcgacggcttccgggacccgttcgccggggcggcggcgagcgagttgtcgctcaggctgcgcgctgggtcctccccgacggcaggtgcggcgagcgtgagcctgccggatcagtcctcggaggtgagctgctccggcttgacgcattggagtagcggcggcggccccggcatgttccagcttccttgcggcggcgccggacaggtggcggtggcggcacgtccgggccccatgcacttctcgcaggtcctgtcgcggtggtccgggtacgcggacgtcacgcagcaggtgttggacgagttcgtcatccgcctgctgcaagacgtggccgggttcgccggcggcggtgaggcgagctgcccgctgccatcgtcaagttattgctccaagacgacgtcatccaacccgaacccgtcggtgtttgttagctcggaagagcaccacaatgagctcaagaacgacctccagaaactgctacagataatggaccaacggtgcaagcaatgcctggacgagatccagaatgcggcgtcgaagtacggcagccttgtgcgcccgggcggcggcggcggcgccctctcgacgctgtttgcgcagcgcgcggtgtcagcgacgcaccggaggctcagggcgcggatcaccggcgagatcgcggcggccacgcggggaggtaatcagccgtcgtcgtcgtcgtcgctgtcgctggccgacagggagcgcagctgggagtcggccttcatccagaagcactgggcgctgcggcagctcaggcgcggcgaccagcaatcgtggcggccgcagcgcggcctgccggagaagtccgtggccgtgctcaaagcctggatgttcgagaacttcctccgcccgtacccgagagacaaggagaaggagatgttagcagccaggagcggactgagcaggagccaggtctccaactggttcatcaacgcccgcgttcggctgtggaagccgatgatagaggagatgtacgaggatctcaagaaggcctccgcaggaataatggaggcctgaacaattaagtcagcagcagggatgcaggtatataaagataagatggctgtttccagatacaagcatcgctgcatgccaccctgcgatctacccatatatatttgtaatctccctggcgtgtttatatagaactgccggagtctgaatcccgtgccggtagtgaagccggatcatcgccagttcatttaatttggcgcttgcatgctgttggtcgagtggaactaagatttcttgttatttatttaacctg

SEQ ID NO: 153 - protein, 474 aa, Zea mays
  misc_feature: Ceres CLONE ID no.354689
MRSCFASGDGSVISSADAPFFHVPEYTLHGGGGFGFGEPAADVAASSFLADGSVLLAGQLLRATALQSVSPEERHGAYYGVTGGSSSYGPSPWDVTVAHAPSRMTKQPVAGEPEGGWIHESSYYCPATWFSGDGFRDPFAGAAASELSLRLRAGSSPTAGAASVSLPDQSSEVSCSGLTHWSSGGGPGMFQLPCGGAGQVAVAARPGPMHFSQVLSRWSGYADVTQQVLDEFVIRLLQDVAGFAGGGEASCPLPSSSYCSKTTSSNPNPSVFVSSEEHHNELKNDLQKLLQIMDQRCKQCLDEIQNAASKYGSLVRPGGGGGALSTLFAQRAVSATHRRLRARITGEIAAATRGGNQPSSSSSLSLADRERSWESAFIQKHWALRQLRRGDQQSWRPQRGLPEKSVAVLKAWMFENFLRPYPRDKEKEMLAARSGLSRSQVSNWFINARVRLWKPMIEEMYEDLKKASAGIMEA

SEQ ID NO: 154 - protein, 539 aa, Oryza sativa subsp. japonica
  misc_feature: Public GI ID no.115445133
MSGNPSFSQLGAVDAAMNGGYFMAASGNGADVPLFHPAMAPPHDHGGSFGYGDAAAAAMDVGAHFAAANNLVLASLATQLFGAAPAAAAHGHGDYLGATTPPEEEMGGGYDVAVGDSSGGAVSLACLGHGQPGDMAAGWCSTSARKPSCNWSSSNAGVHGGSYYLAGVPEAAGFVSAAAAASELSLSLCSKSSSDSMLNAGGDQCSSAASRSGLTQMSRVVVVEPEPPLVPYYPAANFAVVVARSRYAAVAQQVLNDAVGCVLGGVADAAADSASGVDSGSSRPSSCSVAGGAPSSAVSSNNQLIASSGEHTHGGGDASAQRLRSELLTMLQLMDQKYNQCLDEIQSTTARFNTLTHATARAAGMSSSSICAPFAHRAVSAMYHGLRRRIAGEIMSAAAAAGRPCRGGESSSAVTGGERERSWESAFIQKHWAVQQLRRGEQQCWRPQRGLPEKSVAVLKAWMFENFLRPYPKDSEKEMLAARSGLSRNQVSNWFINARVRLWKPMIEEMCEELKRSSGGGAGNQALAMEHMNSQDVVS

SEQ ID NO: 155 - DNA, 1899 bp, Glycine max
  misc_feature: Ceres CLONE ID no.1472219
agttgagtgttcgagagaagcgtctcagagactgtgaaaattcatcaaaatcctctcttacagtgcgtgccacgattcggtccccatcttctctctatctccgtcttgtgcgtcgattttgtctccttcttctcttctactcttcttctcctctattctctcgtcgaggatctcgttttctctcttcgccgccgacttcgattttccagtacccctcctctgttttctcaggatcaagtctctgtgaaagaatctgtctgtaagggaatcaccatgcctctgtttgagcttttcaggctcgccaacgctaatgttgaatatgctcaagacgagaacccttctccacctgtggatgaagttgttgagctggtttgggaaaacggtcagatttcaactcaaagccagtcgagcagaccaagaaacattccaccaccacagtctattcatcaagcaagagctagagagctcggaaatggccccaaggccacgatggtggacgagatccaaatgtcagtgccgtcactaatgaccggtttgagtcaagacgatgacttggttccgtggctgaatcctcatcagtcccttgatggatattgctctgatctcttgcgtgatgcttctcctgttaccgtcaacgagcaagagaccgatgctttcccgagaagaaacaatgggaatgaatcagctcccgctgcttcttcatcacagttcaacggttttgattcacattctctgtatggaaccggtagagctggagatcctgttagccaaccagccaaaccagatcgattcagtcagagactggaaccgttagtaactagtaacaagactggtttgctcaacttttcgcatttcttacgcacggcagctttggctaagactaataataaccctcctggctctaaagagaagagtcctcaaagcccgccgaatgtgtttcagaccagagttcttggagctaaagacaacgagcctaaggataaccaaaaggcttgcttagtttcagaagactcaaacagaaaagagcaagagagtgaaaaggccgttgtatgttcttctgttggctctggtaatagtctcgatggcccatcggaaagtcctttaaaaagaaagcatttggatgttcaagacattgactgtcatagtgaagatgttgagggagaatcaggagatggaagaaaagaagcagctccatctcgaacaagcattggttcaaagagaagccgctcggctgaagtacataatctgtctgaaaggagacggcgtgataggattaacgagaagatgcgtgctcttcaagaactcataccaaactgcaacaaggtggacaaagcttcgatgttagatgaagcaatcgagtatctaaagtcacttcaacttcaagtacagttcatgtcaatggcgtctggttacttcatgccaccggttatgttcccaccgggtatggggcatcattaccaagcagcagcaatggcgatgggcatgggaatgccttatgcaatgggcttgcctgatatgagccgtggaggtccctctgttaacaacagaccacagttccaggttcccgggatgcaacagccagtggccgtggcgcttccacgcgtctctgctggtgggttctatacgggttctgagatgaataagagtgatgatggttcggctagagatctatcaggtactaaagatcaaacgacgacaaaggataacaatagcttgaggccaattaagagaaaacagacgtcttctgatcagttttgtggatcgtcgtgacataaacacaaacttaaggactgatgcattaattattatcaatagcattcaaacaaatcaattctaaatcccacaaatacataaaataaatttgtgttcattacatgttaaaaaagttttttggtgctg

SEQ ID NO: 156 - protein, 498 aa, Glycine max
  misc_feature: Ceres CLONE ID no.1472219
MPLFELFRLANANVEYAQDENPSPPVDEVVELVWENGQISTQSQSSRPRNIPPPQSIHQARARELGNGPKATMVDEIQMSVPSLMTGLSQDDDLVPWLNPHQSLDGYCSDLLRDASPVTVNEQETDAFPRRNNGNESAPAASSSQFNGFDSHSLYGTGRAGDPVSQPAKPDRFSQRLEPLVTSNKTGLLNFSHFLRTAALAKTNNNPPGSKEKSPQSPPNVFQTRVLGAKDNEPKDNQKACLVSEDSNRKEQESEKAVVCSSVGSGNSLDGPSESPLKRKHLDVQDIDCHSEDVEGESGDGRKEAAPSRTSIGSKRSRSAEVHNLSERRRRDRINEKMRALQELIPNCNKVDKASMLDEAIEYLKSLQLQVQFMSMASGYFMPPVMFPPGMGHHYQAAAMAMGMGMPYAMGLPDMSRGGPSVNNRPQFQVPGMQQPVAVALPRVSAGGFYTGSEMNKSDDGSARDLSGTKDQTTTKDNNSLRPIKRKQTSSDQFCGSS

SEQ ID NO: 157 - DNA, 1720 bp, Glycine max
  misc_feature: Ceres CLONE ID no.524419
attctctttttatattcaacaagcaagtttgtctttttttcttctttttttcttctcttctttatggtttttagttactaaaagtgtgtggttttattatgttccagttatggcaaaggttcccttgagctggtatgggaaaatggtcaacttcacgtgcagcaaggaggttcatcatcaacatcaagtgtcgtcacacgaatgactccctcgtgcgaaggttccttcaatgcaaaaagtgcaaggttaagctcactatactccctcatggacttcccagttcaaagggactctgccttggacaattctcaaccaaattctcatcaaagcaatgaccaaaattctggtaagtttgcaaaggctgctaattctagcagcaaaggtcttgatcgttccttagtgataaactcacctaaaggatcaccgggaagacaaaagaaccctttaaattctgatacatcaaacatggttccaagaagtgaggaaacaacacctcctgatgagcaatctgaagctgttggtcatgatagtattcatggatctcgtggtcaatattttaaccaaacctcaagctcagcaagacatagagcaaagggaaaagctcatgatactaaacaaaaatactgtgatgaaggcttgttggaatcttcttccctatgctccattggagcctcaaataacagaaacgtgtgtagcaggacacatgatgatatcgatgactcaacgtatttaagcaataatgatgaagaaccagaagatgtagtgaaggaaaagcctgcttgggaaggcaccggtgtcaagagaagtaggaatgcagaagttcataatttatgtgaaaggaagcgaagagataagatcaacaagaggatgcgtatattgaaggagctcataccaaattgcaataagacggacaaagcttcaatgcttgatgatgccatcgaatatcttaagaccctaaagcttcagttacagatgatgtcaatgggtgctggattttgtatgccgttcatgatgctacctaatgctgctcatcatatgatgaacacaccccatttacatcagttaatgggacttggtatggggttcaggccaggcacagccatgccttgtagcctccctcagttccctattacacctttgcatggtatcactgacaacagagttcacatgtttggtttccctaaccaagttccacccatgccaatttctcatgcacctttcattccaatgcttggaaatccttttacacaacccacccctcttgcaactagcacaaacatcaacctggcggaaaaccctgcttcttcccagttaactactctaatggcctcgtcccccaagaacttgtttattagtggacaagctgaatatgcaacaaagcaataagcaccgagtcaggtttcccttccccactagccatcaatttgtatcccaaaaagagaagaaataaaatttagcatgtctaaggcaattgctagaaaagtcgatttaaagattgtgggatctgataataaatattcagcaactagacactgacacttcatggttggatgctttactgcttaatttgaaacactgatgaaaatggtcattggattcattggaagcatagaagtatggttcttagttatcatccagtaccattctgtatgaattattcaaattaaggtgtcttattaggcgatgaaggcttctaataacaacacaacatttagaagtc

SEQ ID NO: 158 - protein, 394 aa, Glycine max
  misc_feature: Ceres CLONE ID no.524419
MTPSCEGSFNAKSARLSSLYSLMDFPVQRDSALDNSQPNSHQSNDQNSGKFAKAANSSSKGLDRSLVINSPKGSPGRQKNPLNSDTSNMVPRSEETTPPDEQSEAVGHDSIHGSRGQYFNQTSSSARHRAKGKAHDTKQKYCDEGLLESSSLCSIGASNNRNVCSRTHDDIDDSTYLSNNDEEPEDVVKEKPAWEGTGVKRSRNAEVHNLCERKRRDKINKRMRILKELIPNCNKTDKASMLDDAIEYLKTLKLQLQMMSMGAGFCMPFMMLPNAAHHMMNTPHLHQLMGLGMGFRPGTAMPCSLPQFPITPLHGITDNRVHMFGFPNQVPPMPISHAPFIPMLGNPFTQPTPLATSTNINLAENPASSQLTTLMASSPKNLFISGQAEYATKQ

SEQ ID NO: 159 - DNA, 1890 bp, Triticum aestivum
  misc_feature: Ceres CLONE ID no.752318
agtttttcttgacgcacggccgccgtgctcttccttttcttcttggttcttgattcgccggccattgctagcttagcttagtctggtctctggtgtgcgcgcgggcgcgggtactgacgatgatgtgtgcgtgatgctgctgcgcaggagtgagccgggcaacgagctggaggagctgctctgggataacggaccggcgctccggcgggcggcatcgcccttccctcccttctcctgcagcgccgccggcactatgaaggcgcaggagctcgccaagcaccccgcgtcggcgacggcgatggcgcagggacacgacgacgacgccgtgccgtggttgcaacattaccccatcatcggtgtggacgacgacggcagcggcggcgacacggccccgctgccccaggactacttctccaccttgtttcccggtttctccgagctccccgctgcggtggcgagcagagatgccggagcgccatcgacgtctcacgcagcggtcgtcgtgccgccgctgccggagccacctgcgccccccaagcagcagcagcaggcgcctcgatccaccggcgagggcgtcatgaacttctccttcttctcgaggcccctgcagcgctcgctgccgcaggccagcacgagaacgtctgccgcggggagcaagtccgtcgagtcgacgctgctgcagaccaacaggctaagaagcaccccgctgttctcggagcagaggacggcgtggctgcagccgcccaaggagttgcccgccgcggtatccgcatcccgtcctccggcaccgccggtgcctgtccggcatgctgcagaaccggctgcggcgttaccccagagactgcaaccggaagcgagggctgcggcgttaccaccgagattgcaaccggaagtgagggctgcggcgttaccccagagattacaatcggaggcgagggcttccgagatgccaccgccgcccgcaacagcgacgacctcgtctgtctgctccgacaacggtgagcggagccagctgaagaggagcagccaccagactctggagtggtccgtcagtcaggacgacgaggatctcgacgacgaggccggcgggctgagaaggtcggcggcgaggagcaccaagcgcggccgcaccgcggaagtgcacaacatgtcggagaggaggagaagggatcgaatcaacgagaagatgcgcgccctgcaagaactcatccccaactgcaacaagattgacaaggcgtcgatgctggaggaggccatcgagtacctcaagaccctgcagctccaggtccagatgatgtcgacgatggggacggccgggctgtgcatgccgccgatgatggcaatgcagcacatgcagatgccgcccatggcgcacttccaccaccaccacctcggcgccatggggttcggcatgggccccttcgacccgcgcctcgtcgccgccgccggcgccgcgcagttcccttacccgatgatgcccggcgcgcccattttcggcggccacgcgatgcctcccccgccaccgttccaccaggcggcggtggcgggcgctcacatggctcccggccccgtcaccaacgatgccgccaccgcagcgcaggccgagcacgagcagacaccgggcgaccatccgcaggtcccacgaacaatgtaaactagctccaacagcggcggaggtcgacctggctctgtagcaggctaggggcgcacgcagcaaggctcggtaactccgagcagggctgcggcaaaacgtagctgtaaaatctttgtgaaactgtaaaatggttcactgctccggcatcttctccgtgtcagaggataggccgggtacagtgtctgctggagaggatcaatggatcaggatcgcttctgcttcccgctgcagtacac

SEQ ID NO: 160 - protein, 506 aa, Triticum aestivum
  misc_feature: Ceres CLONE ID no.752318
MLLRRSEPGNELEELLWDNGPALRRAASPFPPFSCSAAGTMKAQELAKHPASATAMAQGHDDDAVPWLQHYPIIGVDDDGSGGDTAPLPQDYFSTLFPGFSELPAAVASRDAGAPSTSHAAVVVPPLPEPPAPPKQQQQAPRSTGEGVMNFSFFSRPLQRSLPQASTRTSAAGSKSVESTLLQTNRLRSTPLFSEQRTAWLQPPKELPAAVSASRPPAPPVPVRHAAEPAAALPQRLQPEARAAALPPRLQPEVRAAALPQRLQSEARASEMPPPPATATTSSVCSDNGERSQLKRSSHQTLEWSVSQDDEDLDDEAGGLRRSAARSTKRGRTAEVHNMSERRRRDRINEKMRALQELIPNCNKIDKASMLEEAIEYLKTLQLQVQMMSTMGTAGLCMPPMMAMQHMQMPPMAHFHHHHLGAMGFGMGPFDPRLVAAAGAAQFPYPMMPGAPIFGGHAMPPPPPFHQAAVAGAHMAPGPVTNDAATAAQAEHEQTPGDHPQVPRTM

SEQ ID NO: 161 - DNA, 1967 bp, Zea mays
  misc_feature: Ceres CLONE ID no.1569257
agtctacctctctctctctctcatcacagcacagctccagctctaggctgtagagggtggaagaagcgaggtcgagtcacagaggcctgcagcaaccgtctgcgctgagcatcgaggccacgctctgcagtctgctgtctgctctccctcgataagccgggttccaagctagttctgcagaggaacgaacgaggacgctagtgtgagctctgagctctgactgaacacgaagtgattggcggcttctcatcgatcgatggttgtgttgtgctggccatctgagcgatgaaccagttcgtccctgattggagcaacatgggagacacctccaggccgctcggcgaagaagacgacctcatcgagctgctctggtgcaacggccatgtcgtcatgcagagccagagccaccggaaggtgccgccgaggccggagaaggcggcggcagtggcggctccaccagcccccgcgtccgtgccgcaggaagacgagggcggcctgtggttccccttcgcgctggccgactcgctgaacaaggacatcttctcggagttcttctgcgaggcaccgacgccggcaccggcggcggcggatgcggcgccggcggcctctggtggtggaaccggcaccgaagccggcggcaagtcgtgcggcggggacgtcccagtcccagcagaggacgacaggcgcggcggcggcggggcgtgcgcggtgtcggcgggggacccgtgcgacctgatgccgcctcccaagtcgacgcccgcgtcctgctccaggcagcagacgacgatgagcctggccaacggcggcgacaatgccggaggggacctgccgggcctcgtccgtgcgggggcggaggccggcgcgtcgtcgatgctgagcgcgatcgggtccagcatctgcgggagcaaccaggtcctggtgcagcgcgcggcgtgcgcgccggggcgcgcgtccgcgtccggctccggaaccgcccgcggcgacgggtccgggtccgccgcgctgccgtcggcggtgggcagcgcgaacgcgaacgccgtgggcggcggcaggggccacgaggcgtcctcgtcggggcggtccaactactgctgcttcggcgccgccaccaccacaaccacaaccaccaccacggagcccgcgagcaccagcaaccggagcagcaagcgcaagcggctcgacaccgaggactcggagagtcccagcgaggacgcggagtcggggtccgccgcgatgttggcgcgcaagccgccgcagaagatgacgacggcgcggaggagccgcgccgccgaagtgcacaacctgtcggagcggaggagacgagacaggataaacgagaagatgagagccctgcaagagctcatacctcactgcaacaagacggacaaggcgtcaatgcttgacgaggcgatcgagtacctcaagtcgctgcagctgcaagtgcagatgatgtggatgggcagcgccggcatcgcggcgccgccggcggtgatgttccccggcgtacaccagtacctgcctcggatgggcgtcgggatgggcgcggcagcggcggcggcgctgccgtccatgccgcggctgccgttcatggccccgcaaccggtggtgcccagcgcgccggtgagcgtgggcccggtgccggcctaccggggccacatgcccgcggtgggcatcacggagccgtacgggcactacatcggcgtcaaccacctgcagccggcgccgccgccaccgcaggtccagggcgtgagctactacccgccgccgctgggggcgacggcgaaggccgtgcagcaggctgcagagcttcaccacgtgccggggcccggcggcagcatcatgcccgccggcgccgcacccggagtgctgctcccggagagcgcgcaaggcagagggccagggtgactaacgaccaaaaaggacgtatcacatggctgggccgatgtttaatctgacaagtatggaag

SEQ ID NO: 162 - protein, 539 aa, Zea mays
  misc_feature: Ceres CLONE ID no.1569257
MNQFVPDWSNMGDTSRPLGEEDDLIELLWCNGHVVMQSQSHRKVPPRPEKAAAVAAPPAPASVPQEDEGGLWFPFALADSLNKDIFSEFFCEAPTPAPAAADAAPAASGGGTGTEAGGKSCGGDVPVPAEDDRRGGGGACAVSAGDPCDLMPPPKSTPASCSRQQTTMSLANGGDNAGGDLPGLVRAGAEAGASSMLSAIGSSICGSNQVLVQRAACAPGRASASGSGTARGDGSGSAALPSAVGSANANAVGGGRGHEASSSGRSNYCCFGAATTTTTTTTTEPASTSNRSSKRKRLDTEDSESPSEDAESGSAAMLARKPPQKMTTARRSRAAEVHNLSERRRRDRINEKMRALQELIPHCNKTDKASMLDEAIEYLKSLQLQVQMMWMGSAGIAAPPAVMFPGVHQYLPRMGVGMGAAAAAALPSMPRLPFMAPQPVVPSAPVSVGPVPAYRGHMPAVGITEPYGHYIGVNHLQPAPPPPQVQGVSYYPPPLGATAKAVQQAAELHHVPGPGGSIMPAGAAPGVLLPESAQGRGPG

SEQ ID NO: 163 - DNA, 1861 bp, Panicum virgatum
  misc_feature: Ceres CLONE ID no.1991243
attccttctccgactccgccgcgcgccgcgcggagctcctccttgccacagctcgagctgagctccagctctagagggatatactatacagagcggaaaggcagagtgagaggctgcaagcctgctaggtgtggttgtgctctgctctgctctgctccaccagtcaagcgggatccgaagctcaggagacgaacacggaagctacttgctagtgtgcgctctgattgcgcagaaagtgattggtcggtcatcgatgtttgtggcgatctgagcaatgaaccagttcgtgcctgattggaacagcatgggagacacctccaggccgctcggcgacgacgatgaccttgtcgagctgctctggtgcaacggccatgtcgtcatgcagagccagaacaaccggaagctaccgccgaggcccgagaagccggcgggggcagcagctccagcccccgcgtcggcggcgcaagacgacgaggccggcctctggttcccgttcgcgctggccgactcgctcgacaaggacatcttctcggagttcttctgtgaagcaccagcggcggcggctccggtgcccgcaaccccaggcgtcgaggacggcaagcctgcctgcagggacgtcccgatggaggacgacagcaggcgcggcggggcgtgcgcggtgtccgaggccccgagcgacctgatgccgccgcccaagtcggcgcccgtgtcctggtccaggcagcagacgatgagcctggcgacggacggcggtgacaacgccggcgacctctcggacctcgtccgggcgggcagcgcggggaaggcgtcggcggaggccggcgcgtcgtcgatgctgagcacgatcgggtccagcatctgcgggagcaaccaggagctggtgcagcgcgcggtgagcgcggcgggccgcgtcgcttgcggcggcggctgctccggcagcgcgctgccgtctgcgatgggaagcgcgaacgcgaacgccagaggcaggggcaacgaggccacgtcggggcggtccaactactgcttcggcaccgccgccaccaccaccaccaccgagccgacgagcaccagcaaccggagcagcaagcggaagcggctcgacaccgaggactccgagagccccagcgaggacgcggagtcggagtccgccgcgatgttggcgcgcaagccgccgcagaagctgacgatggcgcggaggagccgcgccgccgaagtgcacaacctctccgagaggaggaggcgggacaggatcaacgagaagatgagagccctgcaagagctgatacctcactgcaacaagaccgacaaggcgtcgatgctggacgaggccatcgagtacctcaagtcgctgcagctgcagctgcagatgatgtggatgggcagcggcatcgcggcgccgccggtgatgttccccggcgtgcaccagtacctgccgcggatgggcgtcgggatgggcgccgcggcgatgccgtccatgccgcggatgccgttcatggcgccgcagccggtggtgcccaacgcgcccgtccacaaccccgtgccgctctcgccggcggcctaccgggggcacatgccggcggtgggcatcaccgagccctacgcgcactacctcggcgtcaaccacctgcagccgacgctgtcccagcatttcgcgcagggcgtgggatactacccgctgggggcgaaggccgccgtgcagcagagtccggcgctccaccacgtgcccggtggcggcatgcccgccgtcgccgccgcgccgggggggctgccgcatgaaaccgctccgagcagaggaccaggatgactatagcaggacaggacaaataatcccctggctgagctgatgttttgacaagtgtgg

SEQ ID NO: 164 - protein, 509 aa, Panicum virgatum
  misc_feature: Ceres CLONE ID no.1991243
MNQFVPDWNSMGDTSRPLGDDDDLVELLWCNGHVVMQSQNNRKLPPRPEKPAGAAAPAPASAAQDDEAGLWFPFALADSLDKDIFSEFFCEAPAAAAPVPATPGVEDGKPACRDVPMEDDSRRGGACAVSEAPSDLMPPPKSAPVSWSRQQTMSLATDGGDNAGDLSDLVRAGSAGKASAEAGASSMLSTIGSSICGSNQELVQRAVSAAGRVACGGGCSGSALPSAMGSANANARGRGNEATSGRSNYCFGTAATTTTTEPTSTSNRSSKRKRLDTEDSESPSEDAESESAAMLARKPPQKLTMARRSRAAEVHNLSERRRRDRINEKMRALQELIPHCNKTDKASMLDEAIEYLKSLQLQLQMMWMGSGIAAPPVMFPGVHQYLPRMGVGMGAAAMPSMPRMPFMAPQPVVPNAPVHNPVPLSPAAYRGHMPAVGITEPYAHYLGVNHLQPTLSQHFAQGVGYYPLGAKAAVQQSPALHHVPGGGMPAVAAAPGGLPHETAPSRGPG

SEQ ID NO: 165 - protein, 345 aa, Oryza sativa subsp. indica
  misc_feature: Public GI ID no.125550778
MNFTFFSRPLQQRPSGGETASASASAAATSTVPVESTVVQAATNRLRSTPLFSDQRMAWLHPPKPSPRAAAPPPPPPLAPTTRHRLDTAAATATVAQRLPPSEARAPDAPPPAATATATTSSVCSGNGDRRQLNWRDSHNNQSAEWSASQDELDLDDELAGVHRRSAARSSKRSRTAEVHNLSERRRRDRINEKMRALQELIPNCNKIDKASMLEEAIEYLKTLQLQVQMMSMGTGMFVPPMMLPAAAAAMQHHHMQMQQMAGPMAAAAHFPHLGAAAAMGLAGFGMPDDAQFPCPMFPAAPPMSMFAPPPPPPPFPHAAATAVEQTPSPPGAADAGNAPAVKQA

SEQ ID NO: 166 - DNA, 2051 bp, Gossypium hirsutum
  misc_feature: Ceres CLONE ID no.1920752
aacttgaagagactcaaatcagagctctagttgtgaagaaatataatccaggtttgctttagtccacaagtagatgggcctactcccttgaacatcttgcagatgggagatgtcataactatacttagcatagaacaactttcttactaaatctctcttctattttatttcatttcttctctctgctacttttcatcctttggtttcttgtataaatagcgtttcccctttcttttttcatgttgtttgatatagctttctaaggcaatctttgggatttcccaaaccaggattttagttgtcttcacactgaactttgctattgttaagtctttttcattttctttctgtagttttgtgcatcttgtttagttgtactggaaagtggaaacggttttcttgggggtcattttctcactcgtatttttgttttattgcagcaaatattttatttgaatctcgagaactccatggctatggtaacattaacatcaaattacaacacaaacctgtcgcaaacattgtcctttgaggaaaaaaatagcagccttcgtgatgtttctttctctactttcttcgatggtgctgacgaaaactatgaaagagaactttcggcttcaaaccgagagctcagctccaagacaaccaacaccaaccaagatgagcatcattatttaggactgaagaaagaagatggagaaattggagtatttggagctgaaaaatacttcaatggaggaattgatttagaaagtccaagaataaacaaaatacatgcaaaaacattggaatgcgttaaagatggtagagtcagcatagagcctgtcaagcctgtaatatatcagggaactccaagtgttcgatcagaatcaagctggaacagccgaagtgcattgctccgaagtactatgagaaatcctcctgggaaaaaacctcctaaagtgaatggaaagagttttctttcgggtcttgctggctgcaaatgttactgttctggtagaaattcagttgagattgaagaagcacaagttggtgaaataagtttcaagagaccagctgctaatggagagggcttgcaaggcaagccaaataaaactgcatccagtaaggcgagcctagaggttaataaaccagtggcggaaccctggacgaaggaagacatttttagtttcccaactatgaattctaataagggaattcgacctgttaaagtgtcactgcaaggagatgtggatgaaatcggacggaaatcattggaggttttcggctcgccggcacttggaaggagaaacaagtctttgaacatcgagaggaggctgcaaatgttctctctggattctaatcccaaagcagaaaaaattgaaaatcctaaaggcaactacaacgatacagagagcgatgcaagttcagacttatttgagatagagagcctcacaggaaaagtcaacccatttcttgtcaaacaaatttctgatgcggcgtctgggtgtgccaccccaacaacttgttatgcaccaagtgaggccagcatagagtggagtgtggtcacagcaagtgcagcagatttctcagtcatgtctgactatgaagagctaaggccacctgtaacttttccaagcccaatgagaacatatccgacacccaccaaaaccaaaggttctaaaaacaagggccgttccagtggtctattggggtgtaacagccaaaaagctgttgaagttgctggagatacacacaaaacaaatgacaaggcaggttttgacccaagaatgcgcagtgtgtctgattcctacatacctgcaacaagatttggagctggtactaaacttgctgcggcttttcaacctactcattcagcaggagcttcacatctcttgtttattcagtagcttttaatagctttccctggaccttattttgtacaacttgtgatgggaaatttcactactataccttatgtttcttttgtatccgtagtagtgttgaattccgtaagtgatgctaattttttagaaagtaaatccaaaaagttcatggggggtcatttgttt

SEQ ID NO: 167 - protein, 472 aa, Gossypium hirsutum
  misc_feature: Ceres CLONE ID no.1920752
MAMVTLTSNYNTNLSQTLSFEEKNSSLRDVSFSTFFDGADENYERELSASNRELSSKTTNTNQDEHHYLGLKKEDGEIGVFGAEKYFNGGIDLESPRINKIHAKTLECVKDGRVSIEPVKPVIYQGTPSVRSESSWNSRSALLRSTMRNPPGKKPPKVNGKSFLSGLAGCKCYCSGRNSVEIEEAQVGEISFKRPAANGEGLQGKPNKTASSKASLEVNKPVAEPWTKEDIFSFPTMNSNKGIRPVKVSLQGDVDEIGRKSLEVFGSPALGRRNKSLNIERRLQMFSLDSNPKAEKIENPKGNYNDTESDASSDLFEIESLTGKVNPFLVKQISDAASGCATPTTCYAPSEASIEWSVVTASAADFSVMSDYEELRPPVTFPSPMRTYPTPTKTKGSKNKGRSSGLLGCNSQKAVEVAGDTHKTNDKAGFDPRMRSVSDSYIPATRFGAGTKLAAAFQPTHSAGASHLLFIQ

SEQ ID NO: 168 - protein, 471 aa, Solanum tuberosum
  misc_feature: Public GI ID no.142942518
MAMVKLEATKSTSSNLLDPSFSSYMINGTEETIVLNLESSRDLSKKVDDGEIDIFSAEKYFNEGVDEENNVTQNKHKIHDDQPVADIVSLQQKIRPLTPSIHSESSWNSRSALLQKVSINHHYQHQHHQPPRPTKTNNKSYGKKFLARFGCNCYCKDKNSVEIDDQLCEKSFKSKSKQNTIKTSTIGANHQNLHFKKIDELGVLGLKSDERFAVPVFDPKVGNPNPGVKIQLHKEEEEESRKSLEVFGFPITEKERSKMSLEKNIGMLTWDAIVPKAEEIDIINIGASSNGTYEEDYAESDASSDLFEIESFPSNNTANPSLVRQGSDSMSCYAPSEVSIDWSVVTASAADFSIMSDIEEVKIPSIRTTSNSNRSVSQNGRDKAKRRSGILLGCNSHKAVGVVGDAYKVSEKSSIEMHQRNFKTYEPIIPMTRFHAESKVNRFDGGNRKHEFTTRSFATTYSGRPADFLYI

SEQ ID NO: 169 - protein, 476 aa, Solanum tuberosum
  misc_feature: Public GI ID no.142942406
MAMVKLEATKSTSTNLLDPSFSAYLINGTEEAIVFNLESSRDLSKKVDDGEIDIFSAEKYFNEGVDEVNNVTQNKLKIHDDQPVAVADIVSLQQKIRPLTPSIHSESSWNSRSALLQKVSRNHHYHQQHHQPPWPTKTNNKSYGKKFLARFGCNCYCKDKNSVEIDDQLGEKSFNRVKYSKSKQNIIKTRSSESSTIGANHQDLHFKKIDELGVLGLKPDERFAVPVFDPKGGIQMKKESEEEESRKSLEVFGFPITEKERSKMSLEKNIGMLTWDAIVPKAEEIDIINIGASSNGTYEEDYAESDASSDLFEIESFPNNNTANPSLVRQGSDSMSCYAPSEVSIDWSVVTASAADFSIMSDIEEVKIPSIRTTSNSNRSVSQNGRDKAKRRSGILLGCNSHKAVGVVGDAYKVSEKSSIEMHQRNFKTYEPIMPMTRFHAESKVNRFDGGNRKHEFTTRSFATTYTRRPADFLYI

SEQ ID NO: 170 - protein, 473 aa, Vitis vinifera
  misc_feature: Public GI ID no.147765302
MDNAANSLHLRDASFSAYLSTAEENFAVKLAESIQSPYLPLTSTQEAAYPVNLGRXMSVEEGELRVFGAEKYFNMKMDDGRRRLTTSSSLXHRQIKEDRADLHCTKPKIRPGTPSASSEASWNSHAALLPRLLRNPSPSKQRKANAKMIFAGFGCNGSCSDKKSVYVDTNAEPGGVHGKEIRKKVAQNDPNPLMLDRTRLSQPGFPVKNEIHSRSFDKASIGCKREEYYGYPIINSGLESLTVKGESVDKRAEEEGRKSLEVFGSHMLKKGDVATNLERKLSVLTWDAIPKVQTVPTTSEGTGXYEDNESDASSDLFEIENLSGSAHPLFNRQASDGMSSCTRYEPKSEASIEWSVVTASAADFSVTSDFDEKKQVESTENSREMNFASTPTRPSKTKSSVGKEAQRSRPTGLLGCRSDKAVRVSEPAYRTNDKVKSDPGWHHRLDSSMPLGKSQGENKVKDFEFPQAQHAFA

SEQ ID NO: 171 - protein, 465 aa, Solanum demissum
  misc_feature: Public GI ID no.47825031
MAMVKLEATKSTSSNLHDPSFSSYMINNTEETIVLNLESSRDLNKKVDDGEIDIFSAEKYFNEGVDEKNNVTQNKHKIHDDQPVADIVSLQQKIRPLTPNIHSESSWNSRSAFLQKVSINHHYQHQHHQPPRPTKTNNKSYGKKFLARFGCNCYCKDKNSVEIDDQLREKSFKSKSKQNTIKTRTIEANHQNLHFKKIDELGVLGLKSDERFTVPVFDPKGGIQMQKETEEEESRKSLEVFGFPITEKERSKMSLEKNIGMLTWDAIVPKAEEIDIINVGASSNGTYEEDYAESDASSDLFEIESFPSNNTANPSLVRQGSDSMSCYAPSEVSIDWSVVTASAADFSIMSDIEEVKIPSIRTTSNSNRSVSQNGRDKAKRRSGILLGCNSHKAVGVVGDAYKVSEKSSIEMHQRSFKTYEPIIPMTRFHAESKVNRFDGGNRKHEFTTRSFATTYSGRPADFLYI

(19) United States
(12) Patent Application Publication
(10) Pub. No.: US 2010/0199378 A1
(43) Pub. Date: Aug. 5, 2010

(54) SHADE TOLERANCE IN PLANTS

(76) Inventors: Shing Kwok, Woodland Hills, CA (US); Kenneth Bounds, Tarzana, CA (US)

Correspondence Address: FISH & RICHARDSON P.C., P.O. BOX 1022, MINNEAPOLIS, MN, 55440-1022, US

(21) Appl. No.: 12/515,687

(22) PCT Filed: Nov. 20, 2007
(86) PCT No.: PCT/US2007/085237

§ 371 Date: Apr. 6, 2010

Related U.S. Application Data
(60) Provisional application No. 60/860,145, filed on Nov. 20, 2006.

(51) Int. Cl. A01H 1/00; A01H 5/00; C12N 5/10; A01C 1/00

(52) U.S. Cl. 800/278; 800/295; 800/298; 800/306; 800/320; 800/314; 800/312; 800/322; 800/305; 435/419; 47/58.1 R

(57) ABSTRACT

Materials and Methods for increasing shade tolerance in plants are disclosed. For example, nucleic acids encoding shade-tolerance polypeptides are disclosed as well as methods for using such nucleic acids to transform plant cells. Also disclosed are plants having increased shade tolerance and plant products produced from plants having increased shade tolerance.

### CROSS-REFERENCE TO RELATED APPLICATION

This application claims priority to U.S. Provisional Patent Application No. 60/860,145, filed on Nov. 20, 2006, and entitled “SHADE TOLERANCE IN PLANTS,” the entire contents of which are incorporated herein by reference.

### TECHNICAL FIELD

This document relates to materials and methods involved in shade tolerance in plants. For example, this document provides plants having increased shade tolerance as well as materials and methods for making plants having increased shade tolerance and plant products derived from plants having increased shade tolerance.

### BACKGROUND

Light is the source of energy that fuels plant growth through photosynthesis. Light is also a developmental signal that modulates morphogenesis, such as de-etiolation and the transition to reproductive development. Since plants cannot choose their surroundings, they are forced to adapt their growth to ambient light conditions and have evolved complex mechanisms for monitoring the quantity and quality of the surrounding light. For example, many kinds of plants respond to growth under dense canopies or at high densities by growing faster and taller (Cerdan and Chory (2003) Nature, 423:881). Densely planted crops tend to place energy into stem and petiole elongation to lift the leaves into the sunlight rather than putting energy into storage or reproductive structures. The response to densely planted crop conditions negatively affects crop yields by reducing the amount of harvestable products such as seeds, fruits and tubers. In addition, tall spindly plants tend to be less wind resistant and lodge more easily, further reducing crop yield.

There is a continuing need for plants that can thrive under less than optimal environmental conditions. One strategy to improve a plant's ability to withstand suboptimal environmental conditions relies upon traditional plant breeding methods. Another approach involves the introduction of exogenous nucleic acids that modify plant responses to suboptimal environmental conditions.

### SUMMARY

The spectral energy distribution of daylight is dramatically altered by vegetation. Light reflected from neighboring vegetation is depleted in red (R) wavelengths, but remains rich in far-red (FR) wavelengths. It is desirable to have plants that exhibit increased shade tolerance. Plants described herein exhibit an increased tolerance to shade conditions, in particular, Short Day plus End-of-Day Far-Red (SD+EODFR) conditions. Wild-type plants typically exhibit shade avoidance responses to SD+EODFR conditions, whereas the SD+EODFR-tolerant plants described herein display a reduction in the level of shade avoidance responses relative to the level of shade avoidance responses displayed by non-SD+EODFR-tolerant plants. Increasing the SD+EODFR tolerance of plants can increase the crop yields of such plants, which can benefit both food consumers and producers.

Provided herein are plants having increased SD+EODFR tolerance. In one aspect, a plant having increased SD+EODFR tolerance can be a plant comprising an exogenous nucleic acid, where the exogenous nucleic acid comprises a nucleotide sequence encoding a polypeptide having 80% or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, where the plant exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In another aspect, a plant having increased SD+EODFR tolerance can be a plant comprising an exogenous nucleic acid comprising a regulatory region operably linked to a nucleotide sequence encoding a polypeptide. The HMM bit score of the amino acid sequence of the polypeptide is greater than about 20, where the HMM is based on the amino acid sequences depicted in one of FIGS. 1-5. The plant exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In yet another aspect, a plant having increased SD+EODFR tolerance can be a plant comprising an exogenous nucleic acid comprising a regulatory region operably linked to a nucleotide sequence having 80 percent or greater sequence identity to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, where the plant exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

Also featured are progeny of any of the plants described above, wherein the progeny has a difference in a response to SD+EODFR light conditions as compared to a corresponding response in a control plant that does not comprise said exogenous nucleic acid.

In another aspect, seeds, vegetative tissue, and fruit from transgenic plants having increased SD+EODFR tolerance are provided. Seeds, vegetative tissue, and fruit can be from a plant comprising an exogenous nucleic acid, where the exogenous nucleic acid comprises a nucleotide sequence encoding a polypeptide having 80% or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, where the plant exhibits a difference in a response to SD+EODFR light conditions as compared to a corresponding response in a control plant that does not comprise the exogenous nucleic acid.

Seeds, vegetative tissue, and fruit can be from a plant comprising an exogenous nucleic acid comprising a regulatory region operably linked to a nucleotide sequence encoding a polypeptide with an HMM bit score of greater than about 20, where the HMM is based on the amino acid sequences depicted in one of FIGS. 1-5, and where the plant exhibits a difference in a response to SD+EODFR light conditions as compared to a corresponding response in a control plant that does not comprise the exogenous nucleic acid.

Seeds, vegetative tissue, and fruit can be from a plant comprising an exogenous nucleic acid comprising a regulatory region operably linked to a nucleotide sequence having 80 percent or greater sequence identity to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, where the plant exhibits a difference in a response to SD+EODFR light conditions as compared to a corresponding response in a control plant that does not comprise the exogenous nucleic acid.

In another aspect, food and feed products comprising seed or vegetative tissue from transgenic plants having increased SD+EODFR tolerance are provided. Food and feed products can comprise seed or vegetative tissue from a plant comprising an exogenous nucleic acid, where the exogenous nucleic acid comprises a nucleotide sequence encoding a polypeptide having 80% or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, where the plant exhibits a difference in a response to SD+EODFR light conditions as compared to a corresponding response in a control plant that does not comprise the exogenous nucleic acid.

Food and feed products can comprise seed or vegetative tissue from a plant comprising an exogenous nucleic acid comprising a regulatory region operably linked to a nucleotide sequence encoding a polypeptide with an HMM bit score of greater than about 20, where the HMM is based on the amino acid sequences depicted in one of FIGS. 1-5, and where the plant exhibits a difference in a response to SD+EODFR light conditions as compared to a corresponding response in a control plant that does not comprise the exogenous nucleic acid.

Food and feed products can comprise seed or vegetative tissue from a plant comprising an exogenous nucleic acid comprising a regulatory region operably linked to a nucleotide sequence having 80 percent or greater sequence identity to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, where the plant exhibits a difference in a response to SD+EODFR light conditions as compared to a corresponding response in a control plant that does not comprise the exogenous nucleic acid.

A method of producing a crop is also provided. In one aspect, the method includes: growing a plurality of plants comprising an exogenous nucleic acid comprising a regulatory region operably linked to a nucleotide sequence encoding a polypeptide having 80 percent or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, where the plants exhibit a difference in a response to SD+EODFR light conditions as compared to a corresponding response in a control plant that does not comprise the exogenous nucleic acid; and harvesting the crop from the plants. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In another aspect, a method of producing a crop includes: growing a plurality of plants comprising an exogenous nucleic acid comprising a regulatory region operably linked to a nucleotide sequence encoding a polypeptide with an HMM bit score of greater than about 20, where the HMM is based on the amino acid sequences depicted in one of FIGS. 1-5, where the plants exhibit a difference in a response to SD+EODFR light conditions as compared to a corresponding response in a control plant that does not comprise the exogenous nucleic acid; and harvesting the crop from the plants. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In yet another aspect, a method of producing a crop includes: growing a plurality of plants comprising an exogenous nucleic acid comprising a regulatory region operably linked to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, where the plants exhibit a difference in a response to SD+EODFR light conditions as compared to a corresponding response in a control plant that does not comprise the exogenous nucleic acid; and harvesting the crop from the plants. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

A method of producing a plant is also provided. In one aspect, the method includes: growing a plant cell comprising an exogenous nucleic acid comprising a regulatory region operably linked to a nucleotide sequence encoding a polypeptide having 80 percent or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, where a plant produced from the cell exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In another aspect, a method of producing a plant includes: growing a plant cell comprising an exogenous nucleic acid comprising a regulatory region operably linked to a nucleotide sequence encoding a polypeptide with an HMM bit score of greater than about 20, where the HMM is based on the amino acid sequences depicted in one of FIGS. 1-5, where a plant produced from the cell exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In another aspect, a method of producing a plant includes growing a plant cell comprising an exogenous nucleic acid comprising a regulatory region operably linked to a nucleotide sequence having 80 percent or greater sequence identity to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, where a plant produced from the cell exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

A method of modulating the SD+EODFR tolerance of a plant is also provided. In one aspect, the method includes: introducing into a plant cell an exogenous nucleic acid comprising a regulatory region operably linked to a nucleotide sequence encoding a polypeptide having 80 percent or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, where a plant produced from the plant cell exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In another aspect, a method of modulating the SD+EODFR tolerance of a plant includes: introducing into a plant cell an exogenous nucleic acid comprising a regulatory region operably linked to a nucleotide sequence encoding a polypeptide with an HMM bit score of greater than about 20, where the HMM is based on the amino acid sequences depicted in one of FIGS. 1-5, where a plant produced from the plant cell exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In another aspect, a method of modulating the SD+EODFR tolerance of a plant includes: introducing into a plant cell an exogenous nucleic acid comprising a regulatory region operably linked to a nucleotide sequence having 80 percent or greater sequence identity to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, where a plant produced from the plant cell exhibits a difference in a response to SD+EODFR light conditions as compared to a corresponding response in a control plant that does not comprise the exogenous nucleic acid. The exogenous nucleic acid can comprise a nucleotide sequence encoding a polypeptide selected from the group consisting of SEQ ID NO:79, SEQ ID NO:87, SEQ ID NO:104, SEQ ID NO:106, and SEQ ID NO:109.

The regulatory region can be a promoter. The promoter can be tissue-preferential, broadly expressing, or inducible.

The plant can be a dicot. The plant can be a member of the genus Brassica, Glycine, Gossypium, Helianthus, Lactuca, or Medicago.

The plant can be a monocot. The plant can be a member of the genus Avena, Cocos, Elaeis, Hordeum, Oryza, Panicum, Secale, Sorghum, Triticum, or Zea.

The difference in a response to SD+EODFR light conditions can be a difference in hypocotyl length. The difference in a response to SD+EODFR light conditions can be a difference in petiole length.

In another aspect, an isolated nucleic acid molecule is provided. The isolated nucleic acid molecule comprises a nucleotide sequence having 95% or greater sequence identity to a nucleotide sequence selected from the group consisting of SEQ ID NO:88, SEQ ID NO:93, SEQ ID NO:110, SEQ ID NO:112, SEQ ID NO:115, SEQ ID NO:119, SEQ ID NO:131, SEQ ID NO:132, SEQ ID NO:134, SEQ ID NO:136, SEQ ID NO:155, SEQ ID NO:157, SEQ ID NO:163, and SEQ ID NO:166.

In another aspect, an isolated nucleic acid is provided. The isolated nucleic acid comprises a nucleotide sequence encoding a polypeptide having 80% or greater sequence identity to an amino acid sequence selected from the group consisting of SEQ ID NO:89, SEQ ID NO:90, SEQ ID NO:94, SEQ ID NO:111, SEQ ID NO:113, SEQ ID NO:116, SEQ ID NO:120, SEQ ID NO:133, SEQ ID NO:135, SEQ ID NO:137, SEQ ID NO:156, SEQ ID NO:158, and SEQ ID NO:167.

### DESCRIPTION OF DRAWINGS

FIG. 1 is an alignment of the amino acid sequence of SEQ ID NO:79 (Ceres Clone ID no. 101035) with homologous and/or orthologous sequences. SEQ ID NO:79 (Ceres Clone ID no. 101035) is a sequence obtained from Arabidopsis thaliana. SEQ ID NO:81 (gi|13752409) is a sequence obtained from Hordeum vulgare subspecies vulgare. SEQ ID NO:84 (Ceres Clone ID no. 398671) is a sequence obtained from Zea mays subspecies mays. SEQ ID NO:80 (gi|92878234) is a sequence obtained from Medicago truncatula. FIG. 1 and the other alignment figures provided herein were generated using the program MUSCLE version 3.52 based on the sequence alignments generated with ProbCon (Do et al., Genome Res., 15(2):330-40 (2005)) version 1.11.

FIG. 2 is an alignment of the amino acid sequence of SEQ ID NO:87 (Ceres ANNOT ID no. 542218) with homologous and/or orthologous sequences. SEQ ID NO:87 (Ceres ANNOT ID no. 542218) is a sequence obtained from Arabidopsis thaliana. SEQ ID NO:92 (gi|62733973) is a sequence obtained from Oryza sativa subspecies japonica. SEQ ID NO:94 (Ceres Clone ID no. 1797005) is a sequence obtained from Panicum virgatum. SEQ ID NO:90 (Ceres Clone ID no. 475075) is a sequence obtained from Glycine max. SEQ ID NO:89 (Ceres ANNOT ID no. 1772685) is a sequence obtained from Populus balsamifera subspecies trichocarpa.

FIG. 3 is an alignment of the amino acid sequence of SEQ ID NO:109 (Ceres ANNOT ID 508164) with homologous and/or orthologous sequences. SEQ ID NO:109 (Ceres ANNOT ID 508164) is a sequence obtained from Arabidopsis thaliana. SEQ ID NO:114 (Ceres Clone ID no. 1580361) is a sequence obtained from Zea mays. SEQ ID NO:113 (Ceres Clone ID no. 1811587) is a sequence obtained from Panicum virgatum. SEQ ID NO:116 (Ceres Clone ID no. 1943506) is a sequence obtained from Gossypium hirsutum. SEQ ID NO:111 (Ceres Clone ID no. 1477240) is a sequence obtained from Populus balsamifera subspecies trichocarpa.

FIG. 4 is an alignment of the amino acid sequence of SEQ ID NO:104 (Ceres ANNOT ID no. 1319615) with homologous and/or orthologous sequences. SEQ ID NO: 104 (Ceres ANNOT ID no. 1319615) is a sequence obtained from Arabidopsis thaliana. SEQ ID NO:156 (Ceres Clone ID no. 1472219) is a sequence obtained from Glycine max. SEQ ID NO:162 (Ceres Clone ID no. 1569257) is a sequence obtained from Zea mays. SEQ ID NO:164 (Ceres Clone ID no. 1991243) is a sequence obtained from Panicum virgatum. SEQ ID NO:160 (Ceres Clone ID no. 752318) is a sequence obtained from Triticum aestivum. SEQ ID NO:165 (gi|125550778) is a sequence obtained from Oryza sativa subspecies indica.

FIG. 5 is an alignment of the amino acid sequence of SEQ ID NO:106 (Ceres ANNOT ID no. 550552) with homologous and/or orthologous sequences. SEQ ID NO: 106 (Ceres ANNOT ID no. 550552) is a sequence obtained from Arabidopsis thaliana. SEQ ID NO:170 (gi|147765302) is a sequence obtained from Vitis vinifera. SEQ ID NO:167 (Ceres Clone ID no. 1920752) is a sequence obtained from Gossypium hirsutum. SEQ ID NO:168 (gi|142942518) is a sequence obtained from Solanum tuberosum. SEQ ID NO:171 (gi|47825031) is a sequence obtained from Solanum demissum.

FIG. 6 is a photograph of a transgenic seedling from event ME04100-01, after five days of growth under SD+EODFR conditions, having a short hypocotyl (right) and a wild-type segregating seedling having a short hypocotyl (left). The meter on the left is marked in millimeter (mm) increments.

### DETAILED DESCRIPTION

This document provides methods and materials related to increasing tolerance to Short Day plus End-of-Day Far-Red (SD+EODFR) conditions in plants. The methods provided herein can include transforming a plant cell with a nucleic acid encoding a polypeptide, wherein expression of the polypeptide results in an increased level of SD+EODFR tolerance. Plant cells produced using such methods can be used to grow plants having increased SD+EODFR tolerance. SD+EODFR-tolerant plants display a reduction in the level of shade avoidance responses relative to the level of shade avoidance responses in non-SD+EODFR-tolerant plants.

### Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include SD+EODFR-tolerance polypeptides. As used herein, SD+EODFR-tolerance polypeptides are polypeptides that, when expressed in a plant, can modulate the tolerance of the plant to SD+EODFR conditions. Modulation of the level of SD+EODFR tolerance can be either an increase or a decrease in the level of SD+EODFR tolerance relative to the corresponding level in a control plant. Such polypeptides typically contain at least one domain indicative of SD+EODFR-tolerance polypeptides, as described in more detail herein. SD+EODFR-tolerance polypeptides typically have an HMM bit score that is greater than 20, as described in more detail herein. In some embodiments, SD+EODFR-tolerance polypeptides have greater than 40% identity to SEQ ID NO:79, SEQ ID NO:87, SEQ ID NO:104, SEQ ID NO:106, and SEQ ID NO:109, as described in more detail herein.

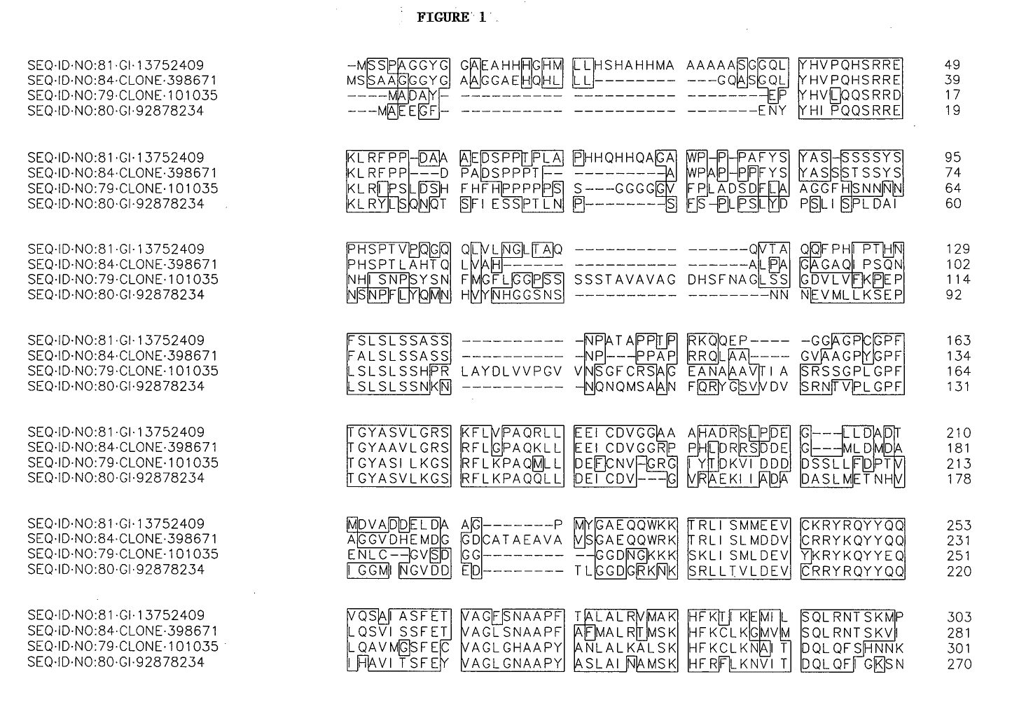

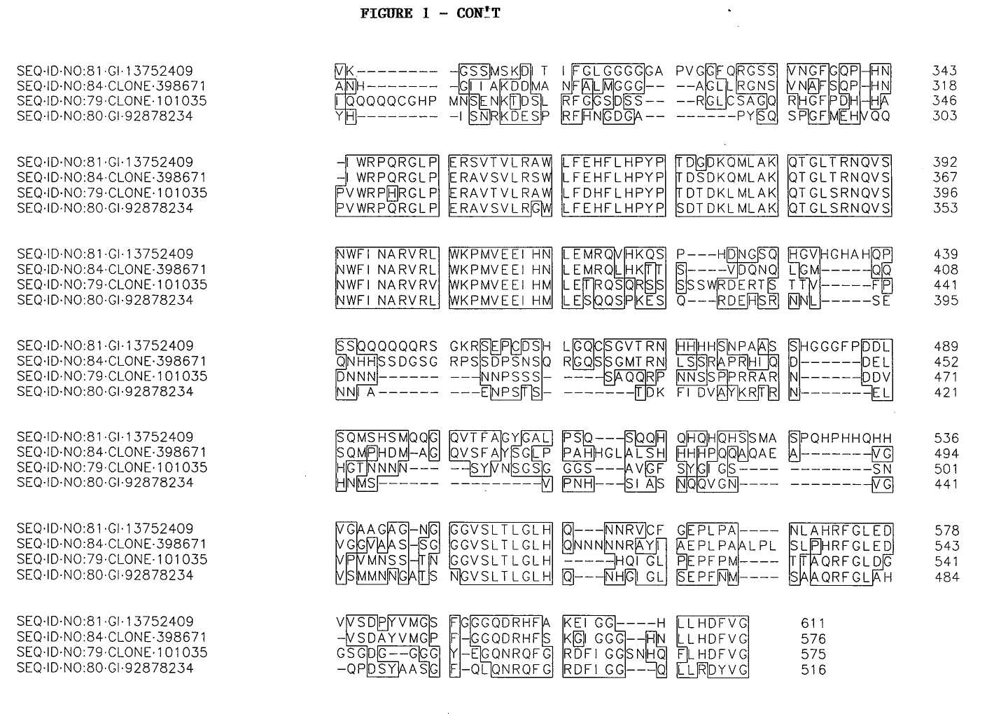

In some embodiments, SD+EODFR-tolerance polypeptide has an amino acid sequence with at least 40% sequence identity, e.g., 50%, 52%, 56%, 59%, 61%, 65%, 70%, 75%, 80%, 85%, 90%, 95%, 97%, 98%, or 99% sequence identity, to one of the amino acid sequences set forth in SEQ ID NO:79, SEQ ID NO:87, SEQ ID NO:104, SEQ ID NO:106, and SEQ ID NO:109. Polypeptides having such a percent sequence identity often have a domain indicative of an SD+EODFR-tolerance polypeptide and/or have an HMM bit score that is greater than 20, as discussed herein. Amino acid sequences of SD+EODFR-tolerance polypeptides having at least 40% sequence identity to one of the amino acid sequences set forth in SEQ ID NO:79, SEQ ID NO:87, SEQ ID NO:104, SEQ ID NO:106, and SEQ ID NO:109 are provided in FIGS. 1-5.

“Percent sequence identity” refers to the degree of sequence identity between any given reference sequence, e.g., SEQ ID NO:79, and a candidate SD+EODFR-tolerance sequence. A candidate sequence typically has a length that is from 80 percent to 200 percent of the length of the reference sequence, e.g., 82, 85, 87, 89, 90, 93, 95, 97, 99, 100, 105, 110, 115, 120, 130, 140, 150, 160, 170, 180, 190, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). Chenna et al., Nucleic Acids Res., 31(13):3497-500 (2003).

ClustalW calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blossom; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

An SD+EODFR-tolerance polypeptide can contain a PDX domain and a homeobox domain. SEQ ID NO:79 sets forth the amino acid sequence of an Arabidopsis clone, identified herein as Ceres Clone ID no. 101035 (Lead 160; At5g02030; SEQ ID NO:78), that is predicted to encode a 575 amino acid polypeptide containing a PDX domain and a homeobox domain. SEQ ID NO:109 sets forth the amino acid sequence of an Arabidopsis clone, identified herein as Ceres ANNOT ID no. 508164 (Lead 204; SEQ ID NO:107), that is predicted to encode a 473 amino acid polypeptide containing a PDX domain.

An SD+EODFR-tolerance polypeptide can comprise the amino acid sequence set forth in SEQ ID NO:79 or SEQ ID NO:109. Alternatively, an SD+EODFR-tolerance polypeptide can be a homolog, ortholog, or variant of the polypeptide having the amino acid sequence set forth in SEQ ID NO:79 or SEQ ID NO:109. For example, an SD+EODFR-tolerance polypeptide can have an amino acid sequence with greater than 40 percent sequence identity, e.g., 41, 42, 45, 47, 50, 52, 55, 57, 60, 65, 70, 75, 80, 85, 90, 95, 98, or 99 percent sequence identity, to the amino acid sequence set forth in SEQ ID NO:79 or SEQ ID NO:109.

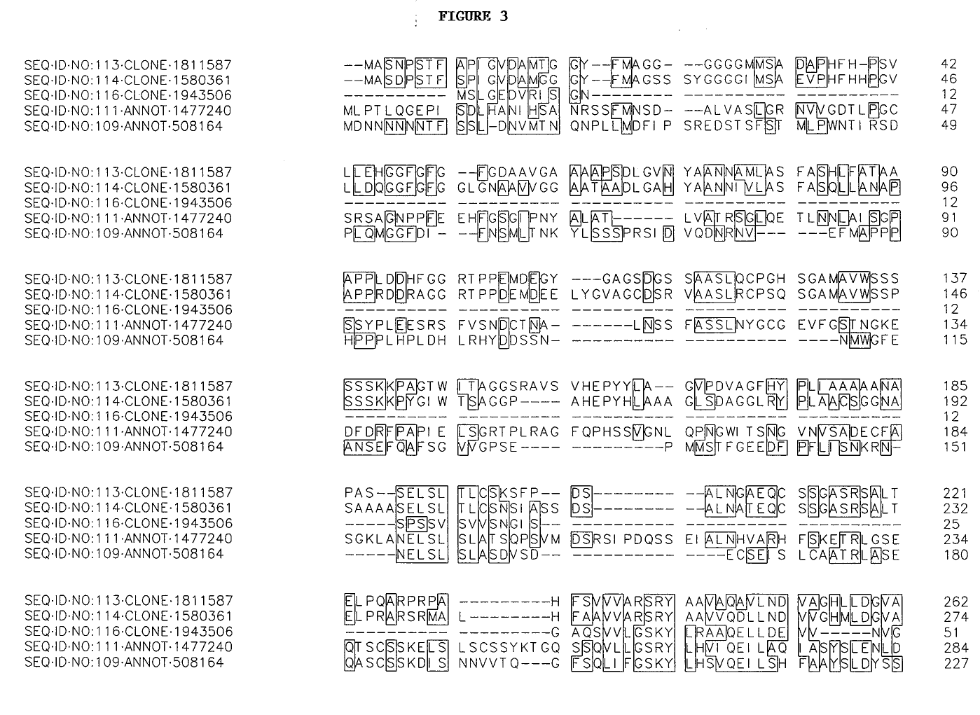

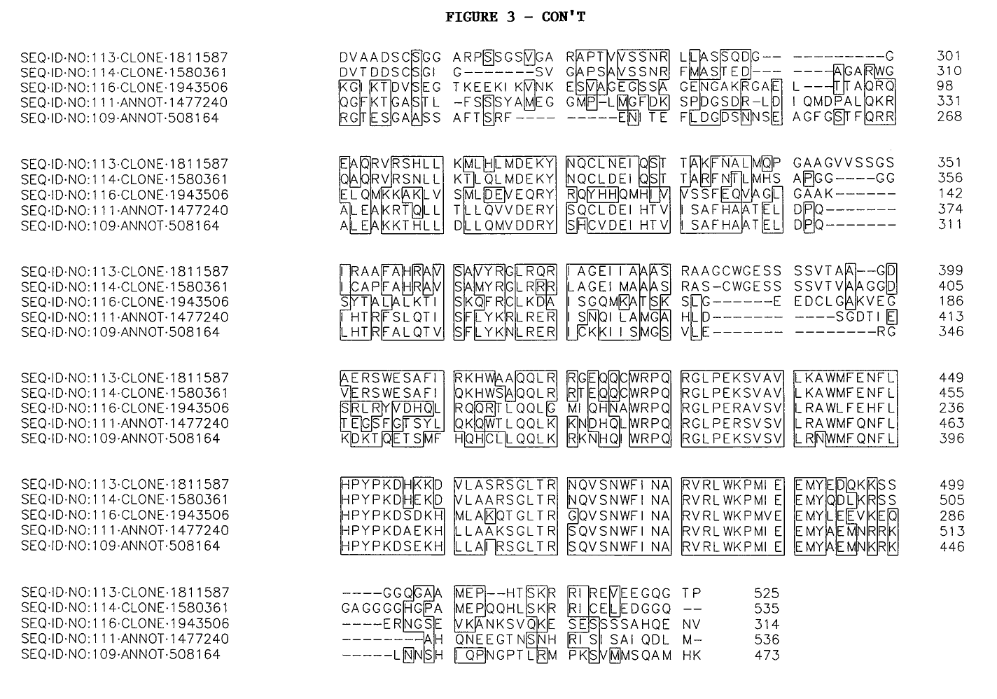

Amino acid sequences of homologs and/or orthologs of the polypeptide having the amino acid sequence set forth in SEQ ID NO:79 and SEQ ID NO:109 are provided in FIGS. 1 and 3, respectively. For example, the alignment in FIG. 1 provides the amino acid sequences of gi|13752409 (SEQ ID NO:81), Ceres CLONE ID no. 398671 (SEQ ID NO:84), and gi|92878234 (SEQ ID NO:80). Other homologs and/or orthologs of SEQ ID NO:79 include gi|19352105 (SEQ ID NO:82), gi|34908294 (SEQ ID NO:83), Ceres CLONE ID no. 1924114 (SEQ ID NO:120), gi|15241667 (SEQ ID NO:121), gi|23397293 (SEQ ID NO:122), Ceres ANNOT ID no. 6039739 (SEQ ID NO:124), gi|147770644 (SEQ ID NO:126), gi|125528380 (SEQ ID NO:127), gi|125552568 (SEQ ID NO:128), gi|115464243 (SEQ ID NO:129),and gi|125594476 (SEQ ID NO:130).

The alignment in FIG. 3 provides the amino acid sequences of Ceres ANNOT ID no. 1477240 (SEQ ID NO:111), Ceres CLONE ID no. 1811587 (SEQ ID NO:113), CLONE ID no. 1580361 (SEQ ID NO:114), and CLONE ID no. 1943506 (SEQ ID NO:116). Other homologs and/or orthologs of SEQ ID NO:109 include gi|15215913 (SEQ ID NO:149), Ceres CLONE ID no. 845859 (SEQ ID NO:151), Ceres CLONE ID no. 354689 (SEQ ID NO:153), and gi|115445133 (SEQ ID NO:154).

In some cases, an SD+EODFR-tolerance polypeptide includes a polypeptide having at least 80 percent sequence identity, e.g., 80, 85, 90, 95, 97, 98, or 99 percent sequence identity, to an amino acid sequence corresponding to SEQ ID NO:80, SEQ ID NO:81, SEQ ID NO:82, SEQ ID NO:83, SEQ ID NO:84, SEQ ID NO:120, SEQ ID NO:121, SEQ ID NO:122, SEQ ID NO:124, SEQ ID NO:126, SEQ ID NO:127, SEQ ID NO:, SEQ ID NO:128, SEQ ID NO:129, SEQ ID NO:130, SEQ ID NO:111, SEQ ID NO:113, SEQ ID NO:114, SEQ ID NO:116, SEQ ID NO:149, SEQ ID NO:151, SEQ ID NO:153, or SEQ ID NO:154.

An SD+EODFR-tolerance polypeptide can contain a DUF525 domain. SEQ ID NO:87 sets forth the amino acid sequence of an Arabidopsis clone, identified herein as Ceres ANNOT ID no. 542218 (Lead 178; At1g06110; SEQ ID NO:85), that is predicted to encode a polypeptide containing a DUF525 domain.

An SD+EODFR-tolerance polypeptide can comprise the amino acid sequence set forth in SEQ ID NO:87. Alternatively, an SD+EODFR-tolerance polypeptide can be a homolog, ortholog, or variant of the polypeptide having the amino acid sequence set forth in SEQ ID NO:87. For example, an SD+EODFR-tolerance polypeptide can have an amino acid sequence with greater than 40 percent sequence identity, e.g., 41, 42, 45, 47, 50, 52, 55, 57, 60, 65, 70, 75, 80, 85, 90, 95, 98, or 99 percent sequence identity, to the amino acid sequence set forth in SEQ ID NO:87.

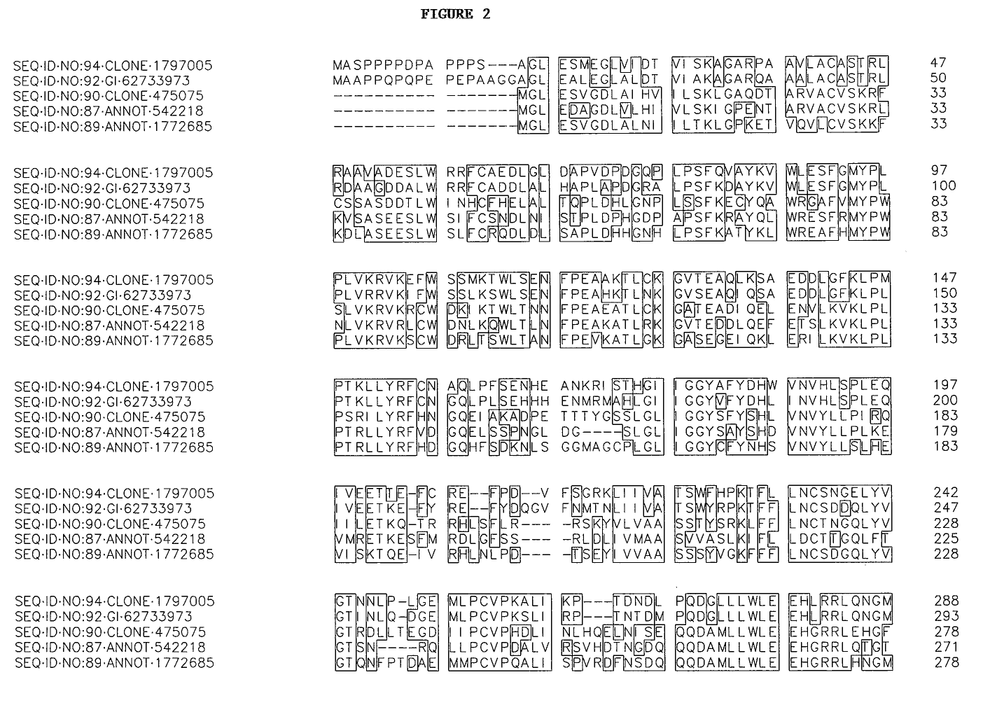

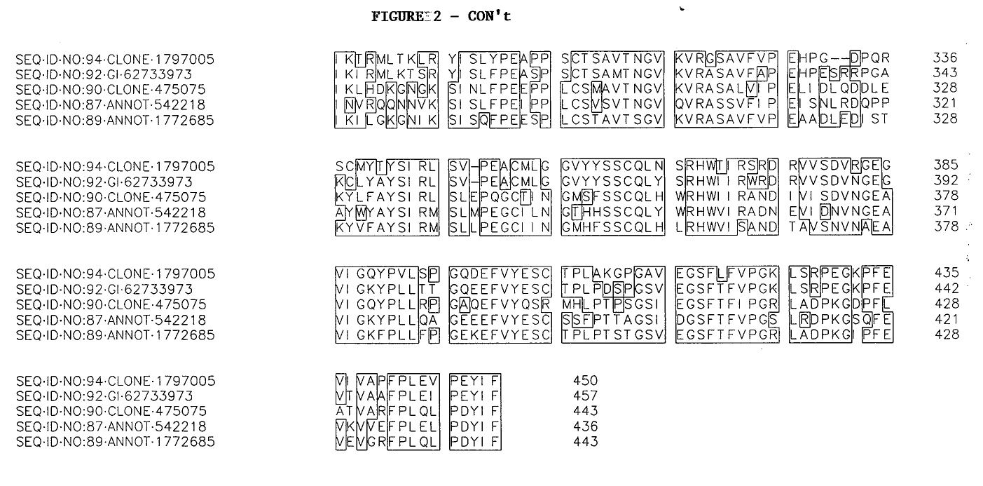

Amino acid sequences of homologs and/or orthologs of the polypeptide having the amino acid sequence set forth in SEQ ID NO:87 are provided in FIG. 2. For example, the alignment in FIG. 2 provides the amino acid sequences of gi|62733973 (SEQ ID NO:92), Ceres Clone ID no. 1797005 (SEQ ID NO:94), Ceres Clone ID no. 475075 (SEQ ID NO:90), and Ceres ANNOT ID no. 1772685 (SEQ ID NO:89). Other homologs and/or orthologs of SEQ ID NO:87 include gi|62733972 (SEQ ID NO:91), Ceres ANNOT ID no. 1455953 (SEQ ID NO:133), Ceres ANNOT ID no. 1541547 (SEQ ID NO:135), Ceres ANNOT ID no. 1488131 (SEQ ID NO:137), Ceres ANNOT ID no. 6098347 (SEQ ID NO:139), gi|125534006 (SEQ ID NO:140), gi|125534002 (SEQ ID NO:141), gi|115485029 (SEQ ID NO:142), gi|125576804 (SEQ ID NO:143), gi|108864217 (SEQ ID NO:144), gi|115485023 (SEQ ID NO:145), and gi|108864214 (SEQ ID NO:146).

In some cases, an SD+EODFR-tolerance polypeptide includes a polypeptide having at least 80 percent sequence identity, e.g., 80, 85, 90, 95, 97, 98, or 99 percent sequence identity, to an amino acid sequence corresponding to SEQ ID NO:89, SEQ ID NO:90, SEQ ID NO:91, SEQ ID NO:92, SEQ ID NO:94, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, and SEQ ID NO:146.

An SD+EODFR-tolerance polypeptide can be a Phytochrome Interacting Factor 3-like 1 (PIL1) helix-loop-helix polypeptide. SEQ ID NO:104 sets forth the amino acid sequence of an Arabidopsis clone, identified herein as Ceres ANNOT ID no. 1319615 (Lead 209; SEQ ID NO:102), that is predicted to encode a 416 amino acid PIL1 helix-loop-helix polypeptide.

An SD+EODFR-tolerance polypeptide can comprise the amino acid sequence set forth in SEQ ID NO:104. Alternatively, an SD+EODFR-tolerance polypeptide can be a homolog, ortholog, or variant of the polypeptide having the amino acid sequence set forth in SEQ ID NO:104. For example, an SD+EODFR-tolerance polypeptide can have an amino acid sequence with greater than 50 percent sequence identity, e.g., 51, 52, 55, 57, 60, 65, 70, 75, 80, 85, 90, 95, 98, or 99 percent sequence identity, to the amino acid sequence set forth in SEQ ID NO:104.

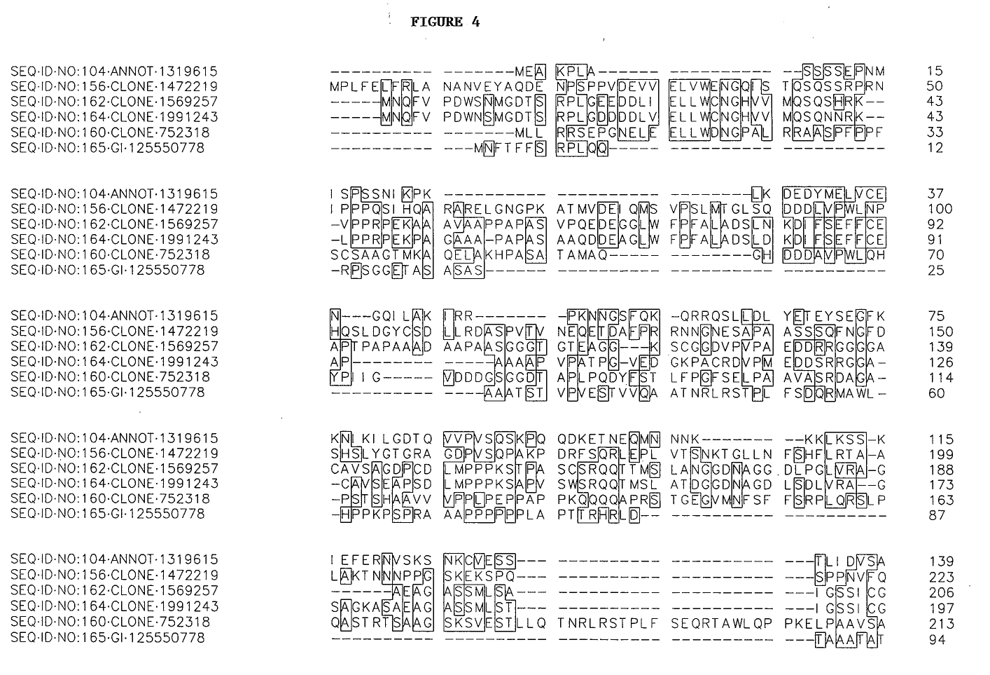

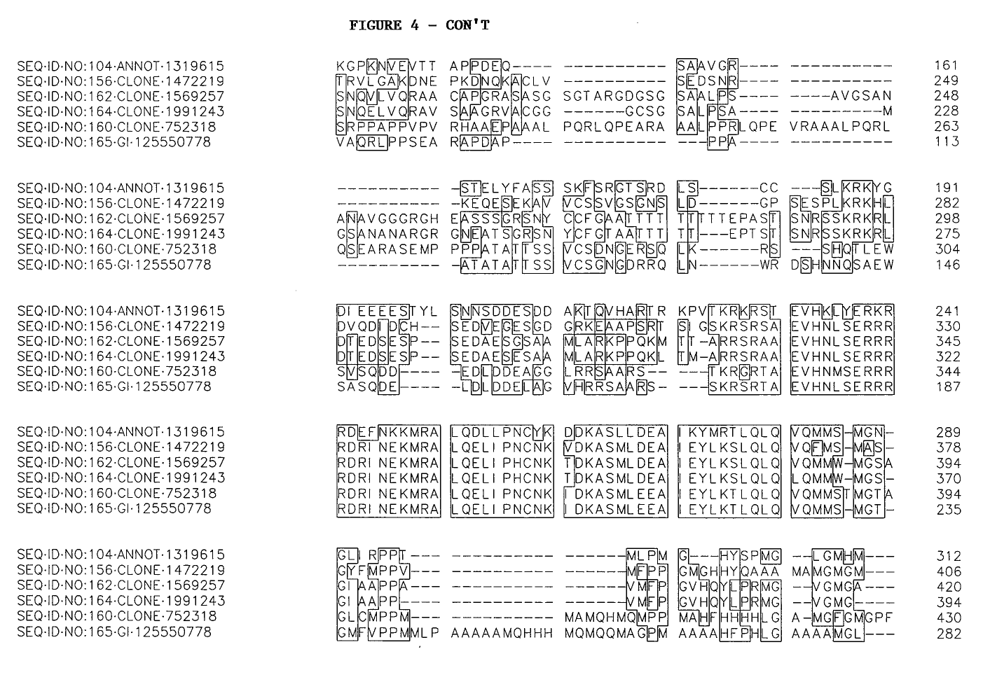

Amino acid sequences of homologs and/or orthologs of the polypeptide having the amino acid sequence set forth in SEQ ID NO:104 are provided in FIG. 4. For example, the alignment in FIG. 4 provides the amino acid sequences of Ceres Clone ID no. 1472219 (SEQ ID NO:156), Ceres Clone ID no. 752318 (SEQ ID NO:160), Ceres Clone ID no. 1569257 (SEQ ID NO:162), Ceres Clone ID no. 1991243 (SEQ ID NO:164), and gi|12550778 (SEQ ID NO:165). Another homolog and/or ortholog of SEQ ID NO:104 includes Ceres Clone ID no. 524419 (SEQ ID NO:158).

In some cases, an SD+EODFR-tolerance polypeptide includes a polypeptide having at least 80 percent sequence identity, e.g., 80, 85, 90, 95, 97, 98, or 99 percent sequence identity, to an amino acid sequence corresponding to SEQ ID NO:156, SEQ ID NO:158, SEQ ID NO:160, SEQ ID NO:162, SEQ ID NO:164, and SEQ ID NO:165.

An SD+EODFR-tolerance polypeptide can be a Phytochrome Kinase Substrate 1 polypeptide. SEQ ID NO:106 sets forth the amino acid sequence of an Arabidopsis clone, identified herein as Ceres ANNOT ID no. 550552 (Lead 210; SEQ ID NO:105), that is predicted to encode a 439 amino acid Phytochrome Kinase Substrate 1 polypeptide. An SD+EODFR-tolerance polypeptide can comprise the amino acid sequence set forth in SEQ ID NO:106. Alternatively, an SD+EODFR-tolerance polypeptide can be a homolog, ortholog, or variant of the polypeptide having the amino acid sequence set forth in SEQ ID NO:106. For example, an SD+EODFR-tolerance polypeptide can have an amino acid sequence with greater than 50 percent sequence identity, e.g., 51, 52, 55, 57, 60, 65, 70, 75, 80, 85, 90, 95, 98, or 99 percent sequence identity, to the amino acid sequence set forth in SEQ ID NO:106.

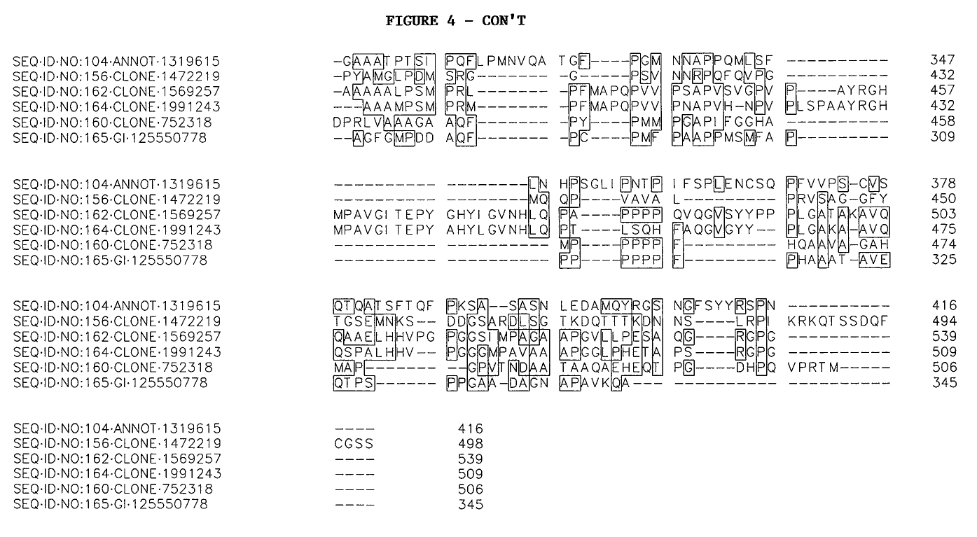

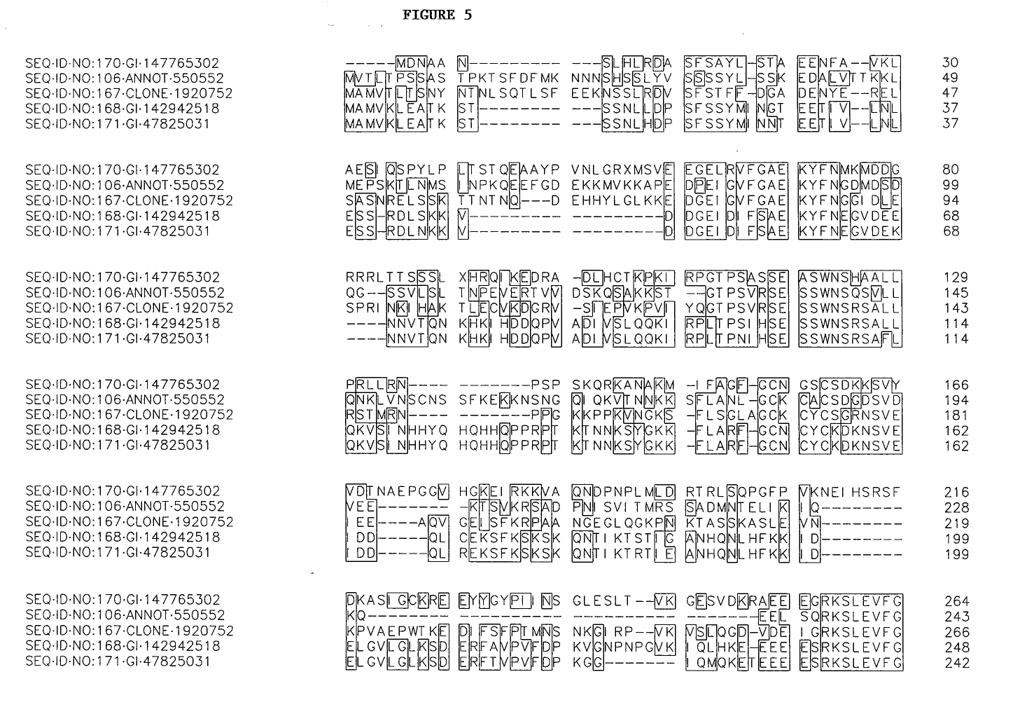

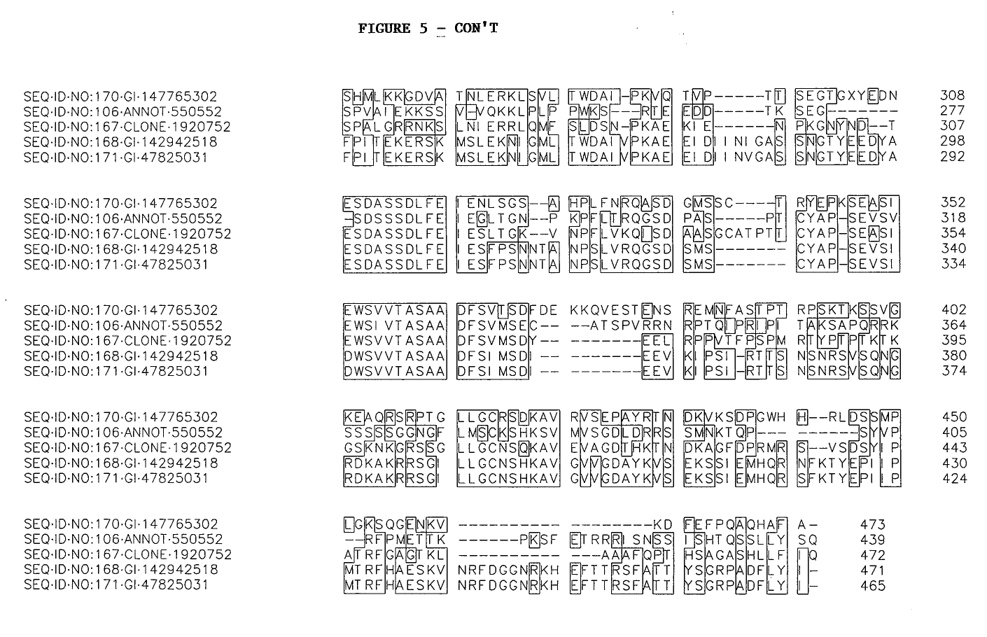

Amino acid sequences of homologs and/or orthologs of the polypeptide having the amino acid sequence set forth in SEQ ID NO:106 are provided in FIG. 5. For example, the alignment in FIG. 5 provides the amino acid sequences of Ceres Clone ID no. 1920752 (SEQ ID NO:167), gi|142942518 (SEQ ID NO:168), gi|147765302 (SEQ ID NO:170), and gi|47825031 (SEQ ID NO:171). Other homologs and/or orthologs of SEQ ID NO:106 include gi|48057594 (SEQ ID NO:117), gi|47824984 (SEQ ID NO:147), and gi|142942406 (SEQ ID NO:169).

In some cases, an SD+EODFR-tolerance polypeptide includes a polypeptide having at least 80 percent sequence identity, e.g., 80, 85, 90, 95, 97, 98, or 99 percent sequence identity, to an amino acid sequence corresponding to SEQ ID NO:117, SEQ ID NO:147, SEQ ID NO:167, SEQ ID NO:168, SEQ ID NO:169, SEQ ID NO:170, and SEQ ID NO:171.

An SD+EODFR-tolerance polypeptide encoded by a recombinant nucleic acid can be a native SD+EODFR-tolerance polypeptide, i.e., one or more additional copies of the coding sequence for an SD+EODFR-tolerance polypeptide that is naturally present in the cell. Alternatively, an SD+EODFR-tolerance polypeptide can be heterologous to the cell, e.g., a transgenic Lycopersicon plant can contain the coding sequence for an SD+EODFR-tolerance polypeptide from a Glycine plant.

An SD+EODFR-tolerance polypeptide can include additional amino acids that are not involved in modulation of SD+EODFR tolerance, and thus can be longer than would otherwise be the case. For example, an SD+EODFR-tolerance polypeptide can include an amino acid sequence that functions as a reporter. Such an SD+EODFR-tolerance polypeptide can be a fusion protein in which a green fluorescent protein (GFP) polypeptide is fused to, e.g., SEQ ID NO:79, or in which a yellow fluorescent protein (YFP) polypeptide is fused to, e.g., SEQ ID NO:156. In some embodiments, an SD+EODFR-tolerance polypeptide includes a purification tag, a chloroplast transit peptide, a mitochondrial transit peptide, an amyloplast peptide, or a leader sequence added to the amino or carboxy terminus.

SD+EODFR-tolerance polypeptide candidates can be identified by analysis of nucleotide and polypeptide sequence alignments. For example, performing a query on a database of nucleotide or polypeptide sequences can identify homologs and/or orthologs of SD+EODFR-tolerance polypeptides. Sequence analysis can involve BLAST, Reciprocal BLAST, or PSI-BLAST analysis of nonredundant databases using known SD+EODFR-tolerance polypeptide amino acid sequences. Those polypeptides in the database that have greater than 40% sequence identity can be identified as candidates for further evaluation for suitability as an SD+EODFR-tolerance polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in SD+EODFR-tolerance polypeptides, e.g., conserved functional domains.

The identification of conserved regions in a template or subject polypeptide can facilitate production of variants of wild type SD+EODFR-tolerance polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Amino acid residues corresponding to Pfam domains included in SD+EODFR-tolerance polypeptides provided herein are set forth in the Sequence Listing. For example, amino acid residues 169 to 292 of the amino acid sequence set forth in SEQ ID NO:79 correspond to a HOX domain, as indicated in fields <222> and <223> for SEQ ID NO:79 in the Sequence Listing.

Variants of SD+EODFR-tolerance polypeptides typically have 10 or fewer conservative amino acid substitutions within the primary amino acid sequence, e.g., 7 or fewer conservative amino acid substitutions, 5 or fewer conservative amino acid substitutions, or between 1 and 5 conservative substitutions. A useful variant polypeptide can be constructed based on one of the alignments set forth in FIG. 1, FIG. 2, FIG. 3, FIG. 4, or FIG. 5. Such a polypeptide includes the conserved regions, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40 percent amino acid sequence identity are useful to identify conserved regions. Conserved regions of related polypeptides can exhibit at least 45 percent amino acid sequence identity (e.g., at least 46 percent, at least 47 percent, at least 48 percent, at least 49 percent, at least 50 percent, at least 53 percent, at least 57 percent, at least 60 percent, at least 65 percent, at least 70 percent, at least 75 percent, at least 80 percent, at least 85 percent, or at least 90 percent amino acid sequence identity). In some embodiments, a conserved region of target and template polypeptides exhibit at least 92, 94, 96, 98, or 99 percent amino acid sequence identity. Amino acid sequence identity can be determined from amino acid or nucleotide sequences. In certain cases, highly conserved domains have been identified within SD+EODFR-tolerance polypeptides. These conserved regions can be useful in identifying functionally similar (orthologous) SD+EODFR-tolerance polypeptides.

In some instances, suitable SD+EODFR-tolerance polypeptides can be synthesized on the basis of consensus functional domains and/or conserved regions in polypeptides that are homologous SD+EODFR-tolerance polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Conserved regions can be identified by homologous polypeptide sequence analysis as described herein. The suitability of polypeptides for use as SD+EODFR-tolerance polypeptides can be evaluated by functional complementation studies.

Functional Homologs Identified by HMMER

In some embodiments, SD+EODFR-tolerance polypeptides include those that fit a Hidden Markov Model based on the polypeptides set forth in any one of FIGS. 1-5. A Hidden Markov Model (HMM) is a statistical model of a consensus sequence for a group of functional homologs. See, Durbin et al., Biological Sequence Analysis: Probabilistic Models of Proteins and Nucleic Acids, Cambridge University Press, Cambridge, UK (1998). An HMM is generated by the program HMMER 2.3.2 with default program parameters, using the sequences of the group of functional homologs as input. The multiple sequence alignment is generated by ProbCons (Do et al., Genome Res., 15(2):330-40 (2005)) version 1.11 using a set of default parameters: -c, --consistency REPS of 2; -ir, --iterative-refinement REPS of 100; -pre, --pre-training REPS of 0. ProbCons is a public domain software program provided by Stanford University.

The default parameters for building an HMM (hmmbuild) are as follows: the default “architecture prior” (archpri) used by MAP architecture construction is 0.85, and the default cutoff threshold (idlevel) used to determine the effective sequence number is 0.62. HMMER 2.3.2 was released Oct. 3, 2003 under a GNU general public license, and is available from various sources on the World Wide Web such as hmmer.janelia.org; hmmer.wustl.edu; and fr.com/hmmer232/. Hmmbuild outputs the model as a text file.

The HMM for a group of functional homologs can be used to determine the likelihood that a candidate SD+EODFR-tolerance polypeptide sequence is a better fit to that particular HMM than to a null HMM generated using a group of sequences that are not structurally or functionally related. The likelihood that a candidate polypeptide sequence is a better fit to an HMM than to a null HMM is indicated by the HMM bit score, a number generated when the candidate sequence is fitted to the HMM profile using the HMMER hmmsearch program. The following default parameters are used when running hmmsearch: the default E-value cutoff (E) is 10.0, the default bit score cutoff (T) is negative infinity, the default number of sequences in a database (Z) is the real number of sequences in the database, the default E-value cutoff for the per-domain ranked hit list (domE) is infinity, and the default bit score cutoff for the per-domain ranked hit list (domT) is negative infinity. A high HMM bit score indicates a greater likelihood that the candidate sequence carries out one or more of the biochemical or physiological function(s) of the polypeptides used to generate the HMM. A high HMM bit score is at least 20, and often is higher. Slight variations in the HMM bit score of a particular sequence can occur due to factors such as the order in which sequences are processed for alignment by multiple sequence alignment algorithms such as the ProbCons program. Nevertheless, such HMM bit score variation is minor.

The SD+EODFR-tolerance polypeptides discussed below fit the indicated HMM with an HMM bit score greater than 20 (e.g., greater than 30, 40, 50, 60, 70, 80, 90, 100, 200, 300, 400, or 500). In some embodiments, the HMM bit score of an SD+EODFR-tolerance polypeptide discussed below is about 50%, 60%, 70%, 80%, 90%, or 95% of the HMM bit score of a functional homolog provided in the Sequence Listing. In some embodiments, an SD+EODFR-tolerance polypeptide discussed below fits the indicated HMM with an HMM bit score greater than 20, and has a domain indicative of an SD+EODFR-tolerance polypeptide. In some embodiments, an SD+EODFR-tolerance polypeptide discussed below fits the indicated HMM with an HMM bit score greater than 20, and has 40% or greater sequence identity (e.g., 55%, 75%, 80%, 85%, 90%, 95%, or 100% sequence identity) to an amino acid sequence shown in any one of FIGS. 1-5.

Polypeptides are shown in the Sequence Listing that have HMM bit scores greater than 650 when fitted to an HMM generated from the amino acid sequences set forth in FIG. 1. Such polypeptides include Ceres Clone ID no. 101035 (SEQ ID NO:79), gi|13752409 (SEQ ID NO:81), Ceres CLONE ID no. 398671 (SEQ ID NO:84), and gi|92878234 (SEQ ID NO:80), gi|19352105 (SEQ ID NO:82), gi|34908294 (SEQ ID NO:83), Ceres CLONE ID no. 1924114 (SEQ ID NO:120), gi|15241667 (SEQ ID NO:121), gi|23397293 (SEQ ID NO:122), Ceres ANNOT ID no. 6039739 (SEQ ID NO:124), gi|147770644 (SEQ ID NO:126), gi|125528380 (SEQ ID NO:127), gi|125552568 (SEQ ID NO:128), gi|115464243 (SEQ ID NO:129),and gi|125594476 (SEQ ID NO:130).

Polypeptides are shown in the Sequence Listing that have HMM bit scores greater than 400 when fitted to an HMM generated from the amino acid sequences set forth in FIG. 2. Such polypeptides include Ceres ANNOT ID no. 542218 (SEQ ID NO:87), gi|62733973 (SEQ ID NO:92), Ceres Clone ID no. 1797005 (SEQ ID NO:94), Ceres Clone ID no. 475075 (SEQ ID NO:90), and Ceres ANNOT ID no. 1772685 (SEQ ID NO:89), gi|62733972 (SEQ ID NO:91), Ceres ANNOT ID no. 1455953 (SEQ ID NO:133), Ceres ANNOT ID no. 1541547 (SEQ ID NO:135), Ceres ANNOT ID no. 1488131 (SEQ ID NO:137), Ceres ANNOT ID no. 6098347 (SEQ ID NO:139), gi|125534006 (SEQ ID NO:140), gi|125534002 (SEQ ID NO:141), gi|115485029 (SEQ ID NO:142), gi|125576804 (SEQ ID NO:143), gi|108864217 (SEQ ID NO:144), gi|115485023 (SEQ ID NO:145), and gi|108864214 (SEQ ID NO:146).

Polypeptides are shown in the Sequence Listing that have HMM bit scores greater than 250 when fitted to an HMM generated from the amino acid sequences set forth in FIG. 3. Such polypeptides include Ceres ANNOT ID no. 508164 (SEQ ID NO:109), Ceres ANNOT ID no. 1477240 (SEQ ID NO:111), Ceres CLONE ID no. 1811587 (SEQ ID NO:113), CLONE ID no. 1580361 (SEQ ID NO:114), and CLONE ID no. 1943506 (SEQ ID NO:116), gi|15215913 (SEQ ID NO:149), Ceres CLONE ID no. 845859 (SEQ ID NO:151), Ceres CLONE ID no. 354689 (SEQ ID NO:153), and gi|115445133 (SEQ ID NO:154).

Polypeptides are shown in the Sequence Listing that have HMM bit scores greater than 40 when fitted to an HMM generated from the amino acid sequences set forth in FIG. 4. Such polypeptides include Ceres ANNOT ID no. 1319615 (SEQ ID NO:104), Ceres Clone ID no. 1472219 (SEQ ID NO:156), Ceres Clone ID no. 752318 (SEQ ID NO:160), Ceres Clone ID no. 1569257 (SEQ ID NO:162), Ceres Clone ID no. 1991243 (SEQ ID NO:164), gi|12550778 (SEQ ID NO:165), and Ceres Clone ID no. 524419 (SEQ ID NO:158).

Polypeptides are shown in the Sequence Listing that have HMM bit scores greater than 950 when fitted to an HMM generated from the amino acid sequences set forth in FIG. 5. Such polypeptides include Ceres ANNOT ID no. 550552 (SEQ ID NO:106), Ceres Clone ID no. 1920752 (SEQ ID NO:167), gi|142942518 (SEQ ID NO:168), gi|147765302 (SEQ ID NO:170), and gi|47825031 (SEQ ID NO:171), gi|48057594 (SEQ ID NO:117), gi|47824984 (SEQ ID NO:147), and gi|142942406 (SEQ ID NO:169).

### Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Recombinant constructs are also provided herein and can be used to transform plants or plant cells in order to increase SD+EODFR tolerance. A recombinant nucleic acid construct comprises a nucleic acid encoding an SD+EODFR-tolerance polypeptide as described herein, operably linked to a regulatory region suitable for expressing the SD+EODFR-tolerance polypeptide in the plant or cell. Thus, a nucleic acid can comprise a coding sequence that encodes any of the SD+EODFR-tolerance polypeptides as set forth SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, or SEQ ID NO:171. Examples of nucleic acids encoding SD+EODFR-tolerance polypeptides are set forth in SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166.

In some cases, a recombinant nucleic acid construct can include a nucleic acid comprising less than the full-length coding sequence of an SD+EODFR-tolerance polypeptide. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given SD+EODFR-tolerance polypeptide can be modified such that optimal expression in a particular plant species is obtained, using appropriate codon bias tables for that species.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

### Regulatory Regions

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a regulatory region, the translation initiation site of the translational reading frame of the polypeptide is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site. A regulatory region typically comprises at least a core (basal) promoter. A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of regulatory regions to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence.

Some suitable regulatory regions initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996).

Examples of various classes of promoters are described below. Some of the promoters indicated below as well as additional promoters are described in more detail in U.S. patent application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 10/950,321; PCT/US05/011105; PCT/US05/034308; and PCT/US05/23639. Nucleotide sequences of promoters are set forth in SEQ ID NOs:1-77, 95-101, and 118. It will be appreciated that a promoter may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:75), YP0144 (SEQ ID NO:54), YP0190 (SEQ ID NO:58), p13879 (SEQ ID NO:74), YP0050 (SEQ ID NO:34), p32449 (SEQ ID NO:76), 21876 (SEQ ID NO:1), YP0158 (SEQ ID NO:56), YP0214 (SEQ ID NO:60), YP0380 (SEQ ID NO:69), PT0848 (SEQ ID NO:26), and PT0633 (SEQ ID NO:7) promoters. Additional examples include the cauliflower mosaic virus (CaMV) 35S promoter, the mannopine synthase (MAS) promoter, the l′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:3), PT0668 (SEQ ID NO:2), PT0886 (SEQ ID NO:29), YP0144 (SEQ ID NO:54), YP0380 (SEQ ID NO:69), and PT0585 (SEQ ID NO:4).

Vascular Tissue Promoters

Examples of promoters that have high or preferential activity in vascular bundles include YP0087 (SEQ ID NO:98), YP0093 (SEQ ID NO:99), YP0108 (SEQ ID NO:100), YP0022 (SEQ ID NO:96), and YP0080 (SEQ ID NO:97). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380 (SEQ ID NO:69), PT0848 (SEQ ID NO:26), YP0381 (SEQ ID NO:70), YP0337 (SEQ ID NO:65), PT0633 (SEQ ID NO:7), YP0374 (SEQ ID NO:67), PT0710 (SEQ ID NO:18), YP0356 (SEQ ID NO:66), YP0385 (SEQ ID NO:72), YP0396 (SEQ ID NO:73), YP0388 (SEQ ID NO:101), YP0384 (SEQ ID NO:71), PT0688 (SEQ ID NO:15), YP0286 (SEQ ID NO:64), YP0377 (SEQ ID NO:68), PD1367 (SEQ ID NO:77), and PD0901 (SEQ ID NO:95). Examples of nitrogen-inducible promoters include PT0863 (SEQ ID NO:27), PT0829 (SEQ ID NO:23), PT0665 (SEQ ID NO:10), and PT0886 (SEQ ID NO:29). Examples of shade-inducible promoters include PR0924 (SEQ ID NO:118), and PT0678 (SEQ ID NO:13).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential, such as PT0678 (SEQ ID NO:13), and senescence-preferential promoters. Promoters designated YP0086 (SEQ ID NO:35), YP0188 (SEQ ID NO:57), YP0263 (SEQ ID NO:61), PT0758 (SEQ ID NO:22), PT0743 (SEQ ID NO:21), PT0829 (SEQ ID NO:23), YP0119 (SEQ ID NO:48), and YP0096 (SEQ ID NO:38), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding an SD+EODFR-tolerance polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### Transgenic Plants and Plant Cells

The invention also features transgenic plant cells and plants comprising at least one recombinant nucleic acid construct described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Transgenic plant cells used in methods described herein can constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively for those species amenable to such techniques. As used herein, a transgenic plant also refers to progeny of an initial transgenic plant provided the progeny inherits the transgene. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant. Seeds produced by a transgenic plant can be grown and then selfed (or outcrossed and selfed) to obtain seeds homozygous for the nucleic acid construct. In some embodiments, transgenic plants exhibiting a desired trait are selected from among independent transformation events.

Transgenic plants can be grown in suspension culture, or tissue or organ culture. For the purposes of this invention, solid and/or liquid tissue culture techniques can be used. When using solid medium, transgenic plant cells can be placed directly onto the medium or can be placed onto a filter that is then placed in contact with the medium. When using liquid medium, transgenic plant cells can be placed onto a flotation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous SD+EODFR-tolerance polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880; 5,204,253; 6,329,571 and 6,013,863. If a cell or cultured tissue is used as the recipient tissue for transformation, plants can be regenerated from transformed cultures if desired, by techniques known to those skilled in the art.

A population of transgenic plants can be screened and/or selected for those members of the population that have a desired trait or phenotype conferred by expression of the transgene. For example, a population of progeny of a single transformation event can be screened for those plants having a desired level of expression of a heterologous SD+EODFR-tolerance polypeptide. As an alternative, a population of plants comprising independent transformation events can be screened for those plants having a desired level of expression of a heterologous SD+EODFR-tolerance polypeptide. Selection and/or screening can be carried out over one or more generations, which can be useful to identify those plants that have a statistically significant difference in a response to SD+EODFR light conditions as compared to a corresponding response in a control plant. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is exhibited by the plant.

### Plant Species

The polynucleotides and vectors described herein can be used to transform a number of monocotyledonous and dicotyledonous plants and plant cell systems, including dicots such as alfalfa, almond, amaranth, apple, apricot, avocado, beans (including kidney beans, lima beans, dry beans, green beans), brazil nut, broccoli, cabbage, canola, carrot, cashew, castor bean, cherry, chick peas, chicory, chocolate, clover, cocoa, coffee, cotton, cottonseed, crambe, eucalyptus, flax, foxglove, grape, grapefruit, hazelnut, hemp, jatropha, jojoba, lemon, lentils, lettuce, linseed, macadamia nut, mango, melon (e.g., watermelon, cantaloupe), mustard, neem, olive, orange, peach, peanut, peach, pear, peas, pecan, pepper, pistachio, plum, poplar, poppy, potato, pumpkin, oilseed rape, quinoa, rapeseed (high erucic acid and canola), safflower, sesame, soaptree bark, soybean, spinach, strawberry, sugar beet, sunflower, sweet potatoes, tea, tomato, walnut, and yams, as well as monocots such as banana, barley, bluegrass, coconut, corn, date palm, fescue, field corn, garlic, millet, oat, oil palm, onion, palm kernel oil, pineapple, popcorn, rice, rye, ryegrass, sorghum, sudangrass, sugarcane, sweet corn, switchgrass, turf grasses, timothy, and wheat. Gymnosperms such as fir, pine, and spruce can also be suitable.

Thus, the methods and compositions described herein can be used with dicotyledonous plants belonging, for example, to the orders Apiales, Arecales, Aristochiales, Asterales, Batales, Campanulales, Capparales, Caryophyllales, Casuarinales, Celastrales, Cornales, Cucurbitales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Linales, Magniolales, Malpighiales, Malvales, Myricales, Myrtales, Nymphaeales, Papaverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Solanales, Trochodendrales, Theales, Umbellales, Urticales, and Violales. The methods and compositions described herein also can be utilized with monocotyledonous plants such as those belonging to the orders Alismatales, Arales, Arecales, Asparagales, Bromeliales, Commelinales, Cyclanthales, Cyperales, Eriocaulales, Hydrocharitales, Juncales, Liliales, Najadales, Orchidales, Pandanales, Poales, Restionales, Triuridales, Typhales, Zingiberales, and with plants belonging to Gymnospermae, e.g., Cycadales, Ephedrales, Ginkgoales, Gnetales, Taxales, and Pinales.

The methods and compositions can be used over a broad range of plant species, including species from the dicot genera Abelmoschus, Acer, Acokanthera, Aconitum, Aesculus, Alangium, Alchornea, Alexa, Alseodaphne, Amaranthus, Ammodendron, Anabasis, Anacardium, Andrographis, Angophora, Anisodus, Apium, Apocynum, Arabidopsis, Arachis, Argemone, Artemisia, Asclepias, Atropa, Azadirachta, Beilschmiedia, Berberis, Bertholletia, Beta, Betula, Bixa, Bleekeria, Borago, Brassica, Calendula, Camellia, Camptotheca, Canarium, Cannabis, Capsicum, Carthamus, Carya, Catharanthus, Centella, Cephaelis, Chelidonium, Chenopodium, Chrysanthemum, Cicer, Cichorium, Cinchona, Cinnamomum, Cissampelos, Citrus, Citrullus, Cocculus, Cocos, Coffea, Cola, Coleus, Convolvulus, Coptis, Corylus, Corymbia, Crambe, Crotalaria, Croton, Cucumis, Cucurbita, Cuphea, Cytisus, Datura, Daucus, Dendromecon, Dianthus, Dichroa, Digitalis, Dioscorea, Duguetia, Erythroxylum, Eschscholzia, Eucalyptus, Euphorbia, Euphoria, Ficus, Fragaria, Galega, Gelsemium, Glaucium, Glycine, Glycyrrhiza, Gossypium, Helianthus, Heliotropium, Hemsleya, Hevea, Hydrastis, Hyoscyamus, Jatropha, Juglans, Lactuca, Landolphia, Lavandula, Lens, Linum, Litsea, Lobelia, Luffa, Lupinus, Lycopersicon, Macadamia, Mahonia, Majorana, Malus, Mangifera, Manihot, Meconopsis, Medicago, Menispermum, Mentha, Micropus, Nicotiana, Ocimum, Olea, Origanum, Papaver, Parthenium, Persea, Petunia, Phaseolus, Physostigma, Pilocarpus, Pistacia, Pisum, Poinsettia, Populus, Prunus, Psychotria, Pyrus, Quillaja, Rabdosia, Raphanus, Rauwolfia, Rhizocarya, Ricinus, Rosa, Rosmarinus, Rubus, Rubia, Salix, Salvia, Sanguinaria, Scopolia, Senecio, Sesamum, Simmondsia, Sinapis, Sinomenium, Solanum, Sophora, Spinacia, Stephania, Strophanthus, Strychnos, Tagetes, Tanacetum, Theobroma, Thymus, Trifolium, Trigonella, Vaccinium, Vicia, Vigna, Vinca, and Vitis; and the monocot genera Agrostis, Allium, Alstroemeria, Ananas, Andropogon, Areca, Arundo, Asparagus, Avena, Cocos, Colchicum, Convallaria, Curcuma, Cynodon, Elaeis, Eragrostis, Erianthus, Festuca, Festulolium, Galanthus, Hemerocallis, Hordeum, Lemna, Lolium, Miscanthus, Musa, Oryza, Panicum, Pennisetum, Phalaris, Phleum, Phoenix, Poa, Ruscus, Saccharum, Secale, Sorghum, Spartina, Triticosecale, Triticum, Uniola, Veratrum, Zea, and Zoysia; and the gymnosperm genera Abies, Cephalotaxus, Cunninghamia, Ephedra, Picea, Pinus, Populus, Pseudotsuga, and Taxus.

In some embodiments, a plant can be from a species selected from Abelmoschus esculentus (okra), Abies, Acer, Allium cepa (onion), Alstroemeria spp., Ananas comosus (pineapple), Andrographis paniculata, Andropogon gerardii (big bluestem), Artemisia annua, Arundo donax (giant reed), Atropa belladonna, Avena sativa, bamboo, bentgrass (Agrostis spp.), Berberis spp., Beta vulgaris (sugarbeet), Bixa orellana, Brassica juncea, Brassica napus (canola), Brassica rapa, Brassica oleracea (broccoli, cauliflower, brusselsprouts), Calendula officinalis, Camellia sinensis (tea), Camptotheca acuminate, Cannabis sativa, Capsicum annum (hot & sweet pepper), Carthamus tinctorius (safflower), Catharanthus roseus, Cephalotaxus spp., Chrysanthemum parthenium, Cinchona officinalis, Citrullus lanatus (watermelon), Coffea arabica (coffee), Colchicum autumnale, Coleus forskohlii, Cucumis melo (melon), Cucumis sativus (cucumber), Cucurbita maxima (squash), Cucurbita moschata (squash), Cynodon dactylon (bermudagrass), Datura stomonium, Dianthus caryophyllus (carnation), Digitalis lanata, Digitalis purpurea, Dioscorea spp., Elaeis guineensis (palm), Ephedra sinica, Ephedra spp., Erianthus spp., Erythroxylum coca, Eucalyptus spp. (eucalyptus), Festuca arundinacea (tall fescue), Fragaria ananassa (strawberry), Galanthus wornorii, Glycine max (soybean), Gossypium hirsutum (cotton), Gossypium herbaceum, Helianthus annuus, (sunflower), Hevea spp. (rubber), Hordeum vulgare, Hyoscyamus spp., Jatropha curcas (jatropha), Lactuca sativa (lettuce), Linum usitatissimum (flax), Lupinus albus (lupin), Lycopersicon esculentum (tomato), Lycopodium serratum (Huperzia serrata), Lycopodium spp., Manihot esculenta (cassava), Medicago sativa (alfalfa), Mentha piperita (mint), Mentha spicata (mint), Miscanthus spp., Miscanthus giganteus (miscanthus), Musa paradisiaca (banana), Nicotiana tabacum (tobacco), Oryza sativa (rice), Panicum spp., Panicum virgatum (switchgrass), Papaver somniferum (opium poppy), Papaver orientale, Parthenium argentatum (guayule), Pennisetum glaucum (pearl millet), Pennisetum purpureum (elephant grass), Petunia spp. (petunia), Phalaris arundinacea (reed canarygrass), Pinus spp. (pine), Poinsettia pulcherrima (poinsettia), Populus spp., Populus balsamifera (poplar), Populus tremuloides (aspen), Rauwolfia serpentina, Rauwolfia spp., Ricinus communis (castor), Rosa spp. (rose), Saccharum spp. (energycane), Salix spp. (willow), Sanguinaria canadensis, Scopolia spp., Secale cereale (rye), Solanum melongena (eggplant), Solanum tuberosum (potato), Sorghum spp., Sorghum almum, Sorghum bicolor (Sorghum), Sorghum halapense, Sorghum vulgare, Spartina pectinata (prairie cordgrass), Spinacea oleracea (spinach), Tanacetum parthenium, Taxus baccata, Taxus brevifolia, Theobroma cacao (cocoa), Triticale (wheat×rye), Triticum aestivum (wheat), Uniola paniculata (oats), Veratrum californica, Vinca rosea, Vitis vinifera (grape), and Zea mays (corn).

### Transgenic Plant Phenotypes

A transformed cell, callus, tissue, or plant can be identified and isolated by selecting or screening the engineered plant material for particular traits or activities, e.g., expression of a selectable marker gene or modulation of SD+EODFR tolerance. Such screening and selection methodologies are well known to those having ordinary skill in the art. In addition, physical and biochemical methods can be used to identify transformants. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are well known.

A population of transgenic plants can be screened and/or selected for those members of the population that have a desired trait or phenotype conferred by expression of a polypeptide described herein. For example, selection and/or screening can be carried out to identify those transgenic plants having a statistically significant difference in a response to SD+EODFR light conditions relative to a control plant that lacks the transgene. Selection and/or screening can be carried out over one or more generations to identify those plants that have the desired trait. Selection and/or screening can also be carried out in more than one geographic location if desired. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is expected to be exhibited by the plant.

Transgenic plants can have an altered phenotype as compared to a corresponding control plant that either lacks the transgene or does not express the transgene. A polypeptide can affect the phenotype of a plant (e.g., a transgenic plant) when expressed in the plant, e.g., at the appropriate time(s), in the appropriate tissue(s), or at the appropriate expression levels. Phenotypic effects can be evaluated relative to a control plant that does not express the exogenous polynucleotide of interest, such as a corresponding wild type plant, a corresponding plant that is not transgenic for the exogenous polynucleotide of interest but otherwise is of the same genetic background as the transgenic plant of interest, or a corresponding plant of the same genetic background in which expression of the polypeptide is suppressed, inhibited, or not induced (e.g., where expression is under the control of an inducible promoter). A plant can be said “not to express” a polypeptide when the plant exhibits less than 10 percent, e.g., less than 9, 8, 7, 6, 5, 4, 3, 2, 1, 0.5, 0.1, 0.01, or 0.001 percent, of the amount of polypeptide or mRNA encoding the polypeptide exhibited by the plant of interest. Expression can be evaluated using methods including, for example, RT-PCR, Northern blots, S1 RNase protection, primer extensions, Western blots, protein gel electrophoresis, immunoprecipitation, enzyme-linked immunoassays, chip assays, and mass spectrometry. It should be noted that if a polypeptide is expressed under the control of a tissue-preferential or broadly expressing promoter, expression can be evaluated in the entire plant or in a selected tissue. Similarly, if a polypeptide is expressed at a particular time, e.g., at a particular time in development or upon induction, expression can be evaluated selectively at a desired time period.

The light in shady environments is enriched in FR wavelengths relative to the light in non-shady environments. Red wavelengths typically range from a photon irradiance of about 630 nm to a photon irradiance of about 700 nm. Far-red wavelengths typically range from a photon irradiance of about 700 nm to a photon irradiance of about 750 nm. The phenotype of a transgenic plant and a corresponding control plant that either lacks the transgene or does not express the transgene can be evaluated under particular environmental conditions that are useful for simulating shade, i.e., Short Day plus End-of-Day Far-Red (SD+EODFR) conditions. SD+EODFR conditions consist of a light period followed by a pulse of far-red-enriched light conditions followed by a 14 hour dark period. The light period is from about 9.0 to about 9.6 hours with a red:far-red ratio of about 5.5, with the following fluence rates: blue₄₅₀=12 μmol/m²/s, red₆₃₃=22 μmol/m²/s, far-red₇₄₀=4 μmol/m²/s, PPFD₄₀₀₋₇₀₀=55 μmol/m²/s. The pulse of far-red-enriched light conditions is from about 0.4 to about 1.0 hours with a red:far-red ratio of about 0.14 with the following fluence rates: blue₄₅₀=0.004 μmol/m²/s, red₆₃₃=10 μmol/m²/s, far-red₇₄₀=70 μmol/m²/s, PPFD₄₀₀₋₇₀₀=8 μmol/m²/s. Sources of lighting equipment appropriate for producing and maintaining SD+EODFR conditions are known to those in art.

As compared to a control plant that does not express an SD+EODFR-tolerance polypeptide grown under SD+EODFR conditions, a transgenic plant expressing an SD+EODFR-tolerance polypeptide can exhibit one or more of the following phenotypes under SD+EODFR conditions: decreases in extension growth, e.g., decreased petiole length, decreased hypocotyl length, decreased internode spacing, and decreased leaf elongation in cereals; acceleration in leaf development, e.g., increased leaf thickness and increased leaf area growth; decreased apical dominance, e.g., increased branching and tillering; increased chloroplast development, e.g., increased chlorophyll synthesis and a change in the balance of the chlorophyll a:b ratio; alterations in flowering and seed/fruit production, e.g., a decreased rate of flowering, an increase in seed set, and an increase of fruit development; and a increase in storage organ deposition.

Typically, a difference (e.g., an increase) in a morphological feature in a transgenic plant or cell relative to a control plant or cell is considered statistically significant at p≦0.05 with an appropriate parametric or non-parametric statistic, e.g., Chi-square test, Student's t-test, Mann-Whitney test, or F-test. In some embodiments, a difference in the dimensions of any individual morphological feature is statistically significant at p<0.01, p<0.005, or p<0.001. A statistically significant difference in, for example, a morphological feature in a transgenic plant compared to the corresponding morphological feature a control plant indicates that expression of the recombinant nucleic acid present in the transgenic plant confers the alteration in the morphological feature.

One suitable phenotype to measure is hypocotyl length. When wild-type seedlings are grown under SD+EODFR conditions, the hypocotyl length is typically significantly increased relative to the hypocotyl length found in wild-type seedlings grown under control light conditions. Thus, seedlings of a transgenic plant and seedlings of a corresponding control plant that either lacks the transgene or does not express the transgene can be grown under SD+EODFR conditions and at the appropriate time, hypocotyl lengths from seedlings of each group can be measured. Under SD+EODFR conditions, a seedling in which the expression of an SD+EODFR-tolerance polypeptide is increased can have a statistically significantly shorter hypocotyl length than a seedling of a corresponding control plant that either lacks the transgene or does not express the transgene.

In some embodiments, under SD+EODFR conditions, a seedling in which expression of an SD+EODFR-tolerance polypeptide is increased can have a shorter hypocotyl length relative to the corresponding control seedlings that either lack the transgene or do not express the transgene. The hypocotyl length can be shorter by at least 20 percent, e.g., 20, 25, 30, 35, 40, 45, 50, 55, 60, 65, 70, 75, or 80 percent, as compared to the hypocotyl length in a corresponding control plant that does not express the transgene.

Another suitable phenotype to measure is petiole length. When wild-type seedlings are grown under SD+EODFR conditions, the petiole length is typically significantly increased relative to the petiole length found in wild-type seedlings grown under non-SD+EODFR conditions. Thus, seedlings of a transgenic plant and seedlings of a corresponding control plant that either lacks the transgene or does not express the transgene can be grown under SD+EODFR conditions and at the appropriate time, petiole lengths from seedlings of each group can be measured. Under SD+EODFR conditions, a seedling in which the expression of an SD+EODFR-tolerance polypeptide is increased can have a statistically significantly shorter petiole length than a seedling of a corresponding control plant that either lacks the transgene or does not express the transgene.

In some embodiments, under SD+EODFR conditions, a seedling in which expression of an SD+EODFR-tolerance polypeptide is increased can have a shorter petiole length relative to the corresponding control seedlings that either lack the transgene or do not express the transgene. The petiole length can be shorter by at least 20 percent, e.g., 20, 25, 30, 35, 40, 45, 50, 55, 60, 65, 70, or 75 percent, as compared to the petiole length in a corresponding control plant that does not express the transgene.

Transgenic plants provided herein have particular uses in agricultural industries. For example, transgenic plants expressing an SD+EODFR-tolerance polypeptide provided herein can maintain development and maturation of such plants under shade conditions, compared to a corresponding control plant. Such a trait can increase plant survival and seedling establishment at high density plant populations in crops even when plants are near mature growth stages. Transgenic plants expressing an SD+EODFR-tolerance polypeptide can be more densely planted than those that are not SD+EODFR-tolerant. Expression of an SD+EODFR-tolerance polypeptide in crop plants can provide increased yields of seed and non-seed tissues from such plants compared to non-SD+EODFR-tolerant plants grown under the same conditions.

The materials and methods described herein are useful for modifying biomass characteristics, such as characteristics of biomass renewable energy source plants. A biomass renewable energy source plant is a plant having or producing material (either raw or processed) that comprises stored solar energy that can be converted to fuel. In general terms, such plants comprise dedicated energy crops as well as agricultural and woody plants. Examples of biomass renewable energy source plants include: switchgrass, elephant grass, giant chinese silver grass, energycane, giant reed (also known as wild cane), tall fescue, bermuda grass, Sorghum, napier grass (also known as uganda grass), triticale, rye, winter wheat, shrub poplar, shrub willow, big bluestem, reed canary grass, and corn.

Information that the polypeptides disclosed herein can increase SD+EODFR tolerance can be useful in breeding of crop plants. Based on the effect of disclosed polypeptides on SD+EODFR tolerance, one can search for and identify polymorphisms linked to genetic loci for such polypeptides. Polymorphisms that can be identified include simple sequence repeats (SSRs), rapid amplification of polymorphic DNA (RAPDs), amplified fragment length polymorphisms (AFLPs) and restriction fragment length polymorphisms (RFLPs).

If a polymorphism is identified, its presence and frequency in populations is analyzed to determine if it is statistically significantly correlated to an alteration in SD+EODFR tolerance. Those polymorphisms that are correlated with an alteration in SD+EODFR tolerance can be incorporated into a marker assisted breeding program to facilitate the development of lines that have a desired alteration in SD+EODFR tolerance. Typically, a polymorphism identified in such a manner is used with polymorphisms at other loci that are also correlated with a desired alteration in SD+EODFR tolerance.

### Articles of Manufacture

Seeds of transgenic plants described herein can be conditioned and bagged in packaging material by means known in the art to form an article of manufacture. Packaging material such as paper and cloth are well known in the art. A package of seed can have a label e.g., a tag or label secured to the packaging material, a label printed on the packaging material, or a label inserted within the package. The package label may indicate that the seed herein incorporates transgenes that provide improved response to shade conditions.

Plants, plant tissues, and/or seeds from plants grown from seeds having an exogenous nucleic acid encoding an SD+EODFR-tolerance polypeptide can be used for making products including, without limitation, human and animal foods, textiles, oils, and/or ethanol

The invention will be further described in the following examples, which do not limit the scope of the invention described in the claims.

### EXAMPLES

### Example 1

### Transgenic Plants

The following symbols are used in the Examples: T₁: first generation transformant; T₂: second generation, progeny of self-pollinated T₁ plants; T₃: third generation, progeny of self-pollinated T₂ plants. Independent transformations are referred to as events.

The following nucleic acids were isolated from Arabidopsis thaliana plants. Ceres Clone ID no. 101035 (SEQ ID NO:78) is a cDNA clone that is predicted to encode a polypeptide having a PDX domain and a homeobox domain (SEQ ID NO:79). Ceres ANNOT ID no. 542218 (SEQ ID NO:85) is a cDNA clone that is predicted to encode polypeptide having the amino acid sequence set forth in SEQ ID NO:87. Ceres ANNOT ID no. 1319615 (SEQ ID NO:102) is a genomic DNA clone that is predicted to encode a PIL1 helix-loop-helix polypeptide (SEQ ID NO:104). Ceres ANNOT ID no. 508164 (SEQ ID NO:107) is a genomic DNA clone that is predicted to encode a polypeptide having a PDX domain (SEQ ID NO:109). Ceres ANNOT ID no. 550552 (SEQ ID NO:105) is a cDNA clone that is predicted to encode Phytochrome Kinase Substrate 1 polypeptide (SEQ ID NO:106).

Nucleic acids having the sequences set forth in SEQ ID NOS:78, 85, and 107 were cloned, using standard molecular biology techniques, into a Ti plasmid vector, CRS338, which encodes a selectable marker gene, phosphinothricin acetyltransferase, that confers Finale® resistance on transformed plants. Constructs were made using the CRS338 vector that contained either Ceres Clone ID no. 101035 (SEQ ID NO:78), Ceres ANNOT ID no. 542218 (SEQ ID NO:85), or Ceres ANNOT ID no. 508164 (SEQ ID NO:107) operably linked in the sense orientation relative to a CaMV 35S constitutive promoter.

Nucleic acids having the sequences set forth in SEQ ID NOS:102 and 105 were cloned, using standard molecular biology techniques, into a Ti plasmid vector, CRS811, which encodes a selectable marker gene, phosphinothricin acetyltransferase, that confers Finale® resistance on transformed plants. Constructs were made using the CRS811 vector that contained either Ceres ANNOT ID no. 1319615 (SEQ ID NO:102) or Ceres ANNOT ID no. 550552 (SEQ ID NO:105) operably linked in the sense orientation relative to a CaMV 35S constitutive promoter.

The constructs were introduced separately into Arabidopsis ecotype Wassilewskija (WS-2) plants by the floral dip method essentially as described in Bechtold, N. et al., C.R. Acad. Sci. Paris, 316:1194-1199 (1993). Two independent transformations were carried out with the CRS338 construct containing Ceres Clone ID no. 101035 (SEQ ID NO:78), resulting in two independent events designated ME04100 and ME03811. A single transformation was carried out with the CRS338 construct containing Ceres ANNOT ID no. 542218 (SEQ ID NO:85), resulting in an event designated ME11961. A single transformation was carried out with the CRS811 construct containing Ceres ANNOT ID no. 1319615 (SEQ ID NO:102), resulting in an event designated ME21198. A single transformation was carried out with the CRS338 construct containing Ceres ANNOT ID no. 508164 (SEQ ID NO:107), resulting in an event designated ME13629. A single transformation was carried out with the CRS811 construct containing Ceres Clone ID no. 550552 (SEQ ID NO:105), resulting in an event designated ME18596. The presence of the vector DNA in each of these events was confirmed by screening the T₁ plants for Finale® resistance. The presence of Ceres Clone DNA in the T₁ plants was confirmed by PCR amplification of insert sequences in DNA extracted from green leaf tissue and the identity of the Ceres Clone was determined by sequencing of the PCR products. Control plants were transformed with either the CRS338 vector lacking inserted Arabidopsis DNA or the CRS811 vector lacking inserted Arabidopsis DNA. T₁ plants were evaluated for morphology and development.

Plants from these independently transformed events were evaluated for their qualitative phenotype according to the methods described in Examples 2 and 3 below. Plants that were attenuated in their shade avoidance response in the T₁ generation, i.e., plants that had reduced hypocotyl length in response to Short Day plus End-of Day-Far-Red (SD+EODFR) assay conditions were selected. T₁ seeds were germinated and allowed to self-pollinate. T₂ seeds were collected and a portion was germinated, allowed to self-pollinate, and T₃ seeds were collected.

### Example 2

### Short Day Plus End-of-Day-Far-Red (SD+EODFR) Assay

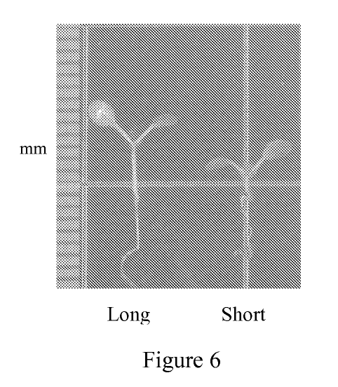

A Short Day plus End-of-Day-Far-Red (SD+EODFR) assay was carried out on seedlings in order to evaluate the effect of SD+EODFR conditions on hypocotyl length. For the SD+EODFR assay, seeds were plated on 0.5% sucrose, 1×MS media (PhytoTech) agar plates, cold-treated for 3-4 days at 4° C., then germinated for 2 days under continuous white light at about 60 μmol/m²/s in walk-in Conviron growth chambers. Seedlings were then exposed to SD+EODFR conditions for 4 days. SD+EODFR conditions were 9.5 hours light, followed by a 30 minute pulse of far-red light at the end of each light cycle, alternating with 14 hours of darkness. Two Gro-Lux (Sylvania, 24660) and two Cool White (Phillips) lights at about 60 μmol/m²/s PPFD, with a red:far-red ratio of about 5.5, were used for the light cycle; the fluence rates under these conditions were: blue₄₅₀=12 μmol/m²/s, red₆₃₃=22 μmol/m²/s, far-red₇₄₀=4 μmol/m²/s, PPFD₄₀₀₋₇₀₀=55 μmol/m²/s. The far-red pulse was generated by 3 SNAP-LITE Far-red light boxes (Quantum devices, SL1515-670-735) at about 8 μmol/m²/s PPFD, with a red:far-red ratio of about 0.14; the fluence rates under these conditions were: blue₄₅₀=0.004 μmol/m²/s, red₆₃₃=10 μmol/m²/s, far-red₇₄₀=70 μmol/m²/s, PPFD₄₀₀₋₇₀₀=8 μmol/m²/s. Control seedlings were cultured exactly as above except that they did not receive the far-red pulse; that is, following germination, they were exposed for two days to a cycle of 10 hours of light alternating with 14 hours of darkness under 2 Gro-Lux and 2 Cool white lights at about 60 μmol/m²/s PPFD, with a red:far-red ratio of about 5.5. Plates were rotated on the third day after plating and hypocotyl length was characterized on the fourth day after plating. The hypocotyls of individual seedlings were determined to be “long” or “short” based on qualitative observation (see, for example, FIG. 6).

Seedlings were then sprayed with sterile Finale® (concentration=0.63%), on two subsequent days, then allowed to grow for 24 hours before chlorophyll fluorescence imaging was done to determine the Finale® resistant:Finale® sensitive ratio. Finale® sensitivity was determined by placing plates of Finale® treated seedlings in a chlorophyll fluorescence imager (CF Imager, Technologica Limited, UK). Finale® resistant seedlings appeared red and Finale® sensitive seedlings appeared blue. Hypocotyl lengths from Finale® resistant seedlings and Finale® sensitive seedlings were then subjected to a Chi-squared analysis to determine statistical significance.

Chi-square analysis of the segregating T₂ seed lines ME03811, ME04100, ME11961, and ME21198 indicated that the bar-gene co-segregated with the trans-gene in a 3:1 ratio indicating a single insertion. Chi-square analysis of the segregating T₂ seed lines ME18596 and ME13629-06 indicated that the bar-gene co-segregated with the transgene in a 2:1 ratio. Chi-square analysis of the segregating T₂ seed line ME13629-02 indicated that the bar-gene co-segregated with the transgene in a 15:1 ratio.

### Example 3

### Analysis of ME04100 Events

The effect of SD+EODFR conditions on hypocotyl length in ME04100 T₂ seedlings was evaluated using the SD+EODFR assay described in Example 2. Control plants for this experiment were T₂ and T₃ segregating progeny that did not contain Ceres Clone ID no. 101035 (SEQ ID NO:78). The T₂ analysis included events ME04100-01, ME04100-02, ME04100-03, and ME04100-04. The T₃ analysis included events ME04100-01-02, ME04100-02-16, ME04100-03-02, and ME04100-04-03. Replicate plates of seeds from each of the ME events and control plants were germinated and grown under either SD+EODFR conditions or control light conditions as described in Example 2.

Results of assays of ME04100 seedlings are shown in Table 1. Under the SD+EODFR conditions, significantly more Finale® resistant T₂ and T₃ seedlings had short hypocotyls than Finale® sensitive seedlings (-segregants). See Table 1. FIG. 3 is a photograph of an example of a transgenic seedling from event ME04100-01 having a short hypocotyl (right) and a wild-type segregating seedling having a long hypocotyl (left).

TABLE 1
Hypocotyl length in seedlings from ME04100

| | Short | Long | Chi- | P-value |
|---|---|---|---|---|
| Line | Hypocotyl | Hypocotyl | Square | vs. -Segregant |
| ME04100-01 T₂ | 56 | 3 | 41.5 | 1.18E−10 |
| ME04100-01 T₂- | 5 | 15 | NA | NA |
| segregant | | | | |
| ME04100-01-02 T₃ | 28 | 5 | 25.67 | 4.06E−07 |
| ME04100-01-02 T₃- | 0 | 11 | NA | NA |
| segregant | | | | |
| ME04100-02 T₂ | 55 | 1 | 27.4 | 1.66E−07 |
| ME04100-02 T₂- | 13 | 12 | NA | NA |
| segregant | | | | |
| ME04100-02-16 T₃ | 38 | 4 | 25.53 | 4.36E−07 |
| ME04100-02-16 T₃- | 3 | 11 | NA | NA |
| segregant | | | | |
| ME04100-03 T₂ | 51 | 4 | 59.48 | 1.24E−14 |
| ME04100-03 T₂- | 1 | 24 | NA | NA |
| segregant | | | | |
| ME04100-03-02 T₃ | 34 | 0 | 41.01 | 1.51E−10 |
| ME04100-03-02 T₃- | 3 | 16 | NA | NA |
| segregant | | | | |
| ME04100-04 T₂ | 57 | 5 | 32.93 | 3.57E−10 |
| ME04100-04 T₂- | 5 | 13 | NA | NA |
| segregant | | | | |
| ME04100-04-02 T₃ | 57 | 4 | 33.13 | 8.61E−09 |
| ME04100-04-02 T₃- | 6 | 13 | NA | NA |
| segregant | | | | |

There were no observable or statistically significant differences between T₂ ME04100 plants and control plants in germination, onset of flowering, rosette area, fertility, and general morphology/architecture.

### Example 4

### Analysis of ME03811 Events

The effect of SD+EODFR conditions on hypocotyl length in ME03811 T₂ and T₃ seedlings was evaluated using the SD+EODFR assay described in Example 2. Control plants for this experiment were ME03811 T₂ and T₃ segregating progeny that did not contain Ceres Clone ID no. 101035 (SEQ ID NO:78). The T₂ analysis included events ME03811-01 and ME03811-03. The T₃ analysis included events ME03811-01-01 and ME03811-03-02. Replicate plates of seeds from each of the ME events and control plants were germinated and grown under either SD+EODFR conditions or control light conditions as described in Example 2.

Results of assays of ME03811 seedlings are shown in Table 2. Under the SD+EODFR conditions, significantly more Finale® resistant T₂ and T₃ seedlings had short hypocotyls than Finale® sensitive seedlings (-segregants).

TABLE 2
Hypocotyl length in seedlings from ME03811

| | Short | Long | Chi- | P-value |
|---|---|---|---|---|
| Line | Hypocotyl | Hypocotyl | Square | vs. -Segregant |
| ME03811-01 T₂ | 60 | 4 | 19.22 | 1.17E−05 |
| ME03811-01 T₂- | 8 | 8 | NA | NA |
| segregant | | | | |
| ME03811-01-01 T₃ | 55 | 5 | 27.62 | 1.48E−07 |
| ME03811-01-01 T₃- | 7 | 13 | NA | NA |
| segregant | | | | |
| ME03811-03 T₂ | 51 | 6 | 32.14 | 1.43E−08 |
| ME03811-03 T₂- | 6 | 17 | NA | NA |
| segregant | | | | |
| ME03811-03-02 T₃ | 59 | 4 | 39.34 | 3.57E−10 |
| ME03811-03-02 T₃- | 4 | 13 | NA | NA |
| segregant | | | | |

There were no observable or statistically significant differences between T₂ ME03811 plants and control plants in germination, onset of flowering, rosette area, fertility, and general morphology/architecture.

### Example 5

### Analysis of ME11961 Events

The effect of SD+EODFR conditions on hypocotyl length in ME11961 T₂ seedlings was evaluated using the SD+EODFR assay described in Example 2. Control plants for this experiment were T₂ and T₃ segregating progeny that did not contain Ceres ANNOT ID no. 542218 (SEQ ID NO:85). The T₂ analysis included events ME11961-03 and ME11961-05. The T₂ analysis included events ME11961-03-05 and ME11961-05-03. Replicate plates of seeds from each of the ME events and control plants were germinated and grown under either SD+EODFR conditions or control light conditions and hypocotyl length was assessed as described in Example 2.

Results of assays of ME11961 seedlings are shown in Table 3. Under the SD+EODFR conditions, significantly more Finale® resistant T₂ and T₃ seedlings had short hypocotyls than Finale® sensitive seedlings (-segregants). See Table 3.

TABLE 3
Hypocotyl length in seedlings from ME11961

| | Short | Long | Chi- | P-value |
|---|---|---|---|---|
| Line | Hypocotyl | Hypocotyl | Square | vs. -Segregant |
| ME11961-03 T₂ | 23 | 1 | 11.96 | 5.421E−04 |
| ME11961-03 T₂- | 5 | 6 | NA | NA |
| segregant | | | | |
| ME11961-03-05 T₃ | 27 | 6 | 8.21 | 4.161E−03 |
| ME11961-03-05 T₃- | 2 | 5 | NA | NA |
| segregant | | | | |
| ME11961-05 T₂ | 26 | 2 | 9.64 | 1.901E−03 |
| ME11961-05 T₂- | 6 | 6 | NA | NA |
| segregant | | | | |
| ME11961-05-03 T₃ | 29 | 4 | 11.65 | 6.42E−04 |
| ME11961-05-03 T₃- | 2 | 5 | NA | NA |
| segregant | | | | |

There were no observable or statistically significant differences between T₂ ME11961 plants and control plants in germination, onset of flowering, rosette area, fertility, and general morphology/architecture.

### Example 6

### Analysis of ME21198 Events

The effect of SD+EODFR conditions on hypocotyl length in ME21198 T₂ seedlings was evaluated using the SD+EODFR assay described in Example 2. Control plants for this experiment were T₂ and T₃ segregating progeny that did not contain Ceres ANNOT ID no. 1319615 (SEQ ID NO:102). The T₂ analysis included events ME21198-02 and ME21198-03. The T₂ analysis included events ME21198-02-06 and ME21198-03-01. Replicate plates of seeds from each of the ME events and control plants were germinated and grown under either SD+EODFR conditions or control light conditions and hypocotyl length was assessed as described in Example 2.

Results of assays of ME21198 seedlings are shown in Table 4. Under the SD+EODFR conditions, significantly more Finale® resistant T₂ and T₃ seedlings had short hypocotyls than Finale® sensitive seedlings (-segregants). See Table 4.

TABLE 4
Hypocotyl length in seedlings from ME21198

| | Short | Long | Chi- | P-value |
|---|---|---|---|---|
| Line | Hypocotyl | Hypocotyl | Square | vs. -Segregant |
| ME21198-02 T₂ | 29 | 3 | 20.83 | 5.01E−06 |
| ME21198-02 T₂- | 1 | 7 | NA | NA |
| segregant | | | | |
| ME21198-02-06 T₃ | 24 | 3 | 17.32 | 3.15E−05 |
| ME21198-02-06 T₃- | 3 | 10 | NA | NA |
| segregant | | | | |
| ME21198-03 T₂ | 28 | 2 | 31.11 | 2.437E−08 |
| ME21198-03 T₂- | 0 | 10 | NA | NA |
| segregant | | | | |
| ME21198-03-01 T₃ | 24 | 4 | 25.71 | 3.959E−07 |
| ME21198-03-01 T₃- | 0 | 12 | NA | NA |
| segregant | | | | |

There were no observable or statistically significant differences between T₂ ME21198 plants and control plants in germination, onset of flowering, rosette area, fertility, and general morphology/architecture.

### Example 7

### Analysis of ME13629 Events

The effect of SD+EODFR conditions on hypocotyl length in ME13629 T₂ seedlings was evaluated using the SD+EODFR assay described in Example 2. Control plants for this experiment were T₂ and T₃ segregating progeny that did not contain Ceres ANNOT ID no. 508164 (SEQ ID NO:107). The T₂ analysis included events ME13629-02 and ME13629-06. The T₂ analysis included events ME13629-02-02 and ME13629-06-02. Replicate plates of seeds from each of the ME events and control plants were germinated and grown under either SD+EODFR conditions or control light conditions and hypocotyl length was assessed as described in Example 2.

Results of assays of ME13629 seedlings are shown in Table 5. Under the SD+EODFR conditions, significantly more Finale® resistant T₂ and T₃ seedlings had short hypocotyls than Finale® sensitive seedlings (-segregants). See Table 5.

TABLE 5
Hypocotyl length in seedlings from ME13629

| | Short | Long | Chi- | P-value |
|---|---|---|---|---|
| Line | Hypocotyl | Hypocotyl | Square | vs. -Segregant |
| ME13629-02 T₂ | 32 | 7 | 4.10 | 4.28E−02 |
| ME13629-02 T₂- | 0 | 1 | NA | NA |
| segregant | | | | |
| ME13629-02-02 T₃ | 22 | 2 | 13.41 | 2.5E−04 |
| ME13629-02-02 T₃- | 2 | 5 | NA | NA |
| segregant | | | | |
| ME13629-06 T₂ | 32 | 1 | 21.19 | 4.159E−06 |
| ME13629-06 T₂- | 2 | 5 | NA | NA |
| segregant | | | | |
| ME13629-06-02 T₃ | 20 | 4 | 6.86 | 8.83E−03 |
| ME13629-06-02 T₃- | 7 | 9 | NA | NA |
| segregant | | | | |

There were no observable or statistically significant differences between T₂ ME13629 plants and control plants in germination, onset of flowering, rosette area, fertility, and general morphology/architecture.

### Example 8

### Analysis of ME18596 Events

The effect of SD+EODFR conditions on hypocotyl length in ME18596 T₂ seedlings was evaluated using the SD+EODFR assay described in Example 2. Control plants for this experiment were T₂ and T₃ segregating progeny that did not contain Ceres ANNOT ID no. 550552 (SEQ ID NO:105). The T₂ analysis included events ME18596-03 and ME18596-05. The T₂ analysis included events ME18596-03-04 and ME18596-05-06. Replicate plates of seeds from each of the ME events and control plants were germinated and grown under either SD+EODFR conditions or control light conditions and hypocotyl length was assessed as described in Example 2.

Results of assays of ME18596 seedlings are shown in Table 6. Under the SD+EODFR conditions, significantly more Finale® resistant T₂ and T₃ seedlings had short hypocotyls than Finale® sensitive seedlings (-segregants). See Table 6.

TABLE 6
Hypocotyl length in seedlings from ME18596

| | Short | Long | Chi- | P-value |
|---|---|---|---|---|
| Line | Hypocotyl | Hypocotyl | quare | vs. -Segregant |
| ME18596-03 T₂ | 18 | 0 | 7.62 | 5.78E−03 |
| ME18596-03 T₂- | 9 | 5 | NA | NA |
| segregant | | | | |
| ME18596-03-04 T₃ | 18 | 6 | 5.63 | 1.77E−02 |
| ME18596-03-04 T₃- | 6 | 10 | NA | NA |
| segregant | | | | |
| ME18596-05 T₂ | 25 | 3 | 5.85 | 1.56E−02 |
| ME18596-05 T₂- | 6 | 5 | NA | NA |
| segregant | | | | |
| ME18596-05-06 T₃ | 20 | 6 | 4.64 | 3.12E−02 |
| ME18596-05-06 T₃- | 6 | 8 | NA | NA |
| segregant | | | | |

There were no observable or statistically significant differences between T₂ ME18596 plants and control plants in germination, onset of flowering, rosette area, fertility, and general morphology/architecture.

### Example 9

### Determination of Functional Homolog and/or Orthologue Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify polypeptides having BLAST sequence identity of 80 percent or greater to the query polypeptide and an alignment length of 85 percent or greater along the shorter sequence in the alignment. The query polypeptide and any of the aforementioned identified polypeptides were designated as a cluster.

The BLASTP version 2.0 program from Washington University at Saint Louis, Mo., USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps were excluded.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and a sequence identity cutoff of 35 percent. Among the top hits, the sequence having the lowest E-value was designated as the best hit, and considered a potential functional homolog or ortholog. Any other top hit that had a sequence identity of 80 percent or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:79, SEQ ID NO:87, SEQ ID NO:109, SEQ ID NO:104, and SEQ ID NO:106 are shown in FIGS. 1-5, respectively.

### Example 10

### Determination of Functional Homologs by Hidden Markov Models

Hidden Markov Models (HMMs) were generated by the program HMMER 2.3.2. To generate each HMM, the default HMMER 2.3.2 program parameters, configured for glocal alignments, were used.

An HMM was generated using the sequences shown in FIG. 1 as input. These sequences were fitted to the model and a representative HMM bit score for each sequence is shown in the Sequence Listing. Additional sequences were fitted to the model, and representative HMM bit scores for any such additional sequences are shown in the Sequence Listing. The results indicate that these additional sequences are functional homologs of SEQ ID NO:79.

The procedure above was repeated and an HMM was generated for each group of sequences shown in FIGS. 2-5, using the sequences shown in each Figure as input for that HMM. A representative bit score for each sequence is shown in the Sequence Listing. Additional sequences were fitted to certain HMMs, and representative HMM bit scores for such additional sequences are shown in the Sequence Listing. The results indicate that these additional sequences are functional homologs of the sequences used to generate that HMM.

### Example 11

### Characterization of the SD+EODFR Tolerance of Seedlings from Orthologous Sequence Events

Ceres CLONE ID no. 1472219 (SEQ ID NO:155) was isolated from Arabidopsis thaliana and is predicted to encode a 498 amino acid polypeptide (SEQ ID NO:156).

Ceres CLONE ID no. 1472219 was cloned into a Ti plasmid vector, CRS811, containing a phosphinothricin acetyltransferase gene, which confers Finale® resistance to transformed plants. Ceres CLONE ID no. 1472219 was operably linked to a CaMV 35S promoter in the constructs made using the CRS811 vector. Wild-type Arabidopsis thaliana ecotype Wassilewskija (Ws) plants were transformed with the construct. The transformation was performed essentially as described in Bechtold and Pelletier, Methods Mol Biol., 82:259-66 (1998).

A transgenic Arabidopsis line containing Ceres CLONE ID no. 1472219 was designated ME29406. The presence of a vector containing Ceres CLONE ID no. 1472219 in the transgenic Arabidopsis line transformed with the vector was confirmed by Finale® resistance, polymerase chain reaction (PCR) amplification from green leaf tissue extract, and sequencing of PCR products.

T₂ seedlings from event −01 ME29406 were grown under SD+EODFR conditions and evaluated for hypocotyl length as described in Example 2. A Chi-square test was performed to compare transgenic seedlings and corresponding non-transgenic segregants having a short or a long hypocotyl, as described in Example 3. Seedlings from event −01 ME29406 displayed a short hypocotyl under SD+EODFR conditions, and the transgene was linked to the short hypocotyl phenotype with a confidence level of p<0.05 (Table 7).

TABLE 7
Hypocotyl length in seedlings from ME29406

| | Homolog/ | | | | |
|---|---|---|---|---|---|
| | ortholog | Short | Long | Chi- | |
| Event | of | Hypocotyl | Hypocotyl | Square | p-value |
| T₂ seedlings | ME21198 | 23 | 7 | 4.48 | 3.42E−02 |
| from event -01 | (SEQ ID | | | | |
| of ME29406 | NO: 104) | | | | |
| T₂ non- | | 3 | 5 | | |
| transgenic | | | | | |
| segregants of | | | | | |
| event -01 of | | | | | |
| ME29406 | | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A plant comprising an exogenous nucleic acid, said exogenous nucleic acid comprising a nucleotide sequence encoding a polypeptide having 80% or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, wherein said plant exhibits a difference in a response to SD+EODFR light conditions as compared to a corresponding response in a control plant that does not comprise said exogenous nucleic acid.

2. (canceled)

3. A plant comprising an exogenous nucleic acid, said exogenous nucleic acid comprising a regulatory region operably linked to a nucleotide sequence having 80 percent or greater sequence identity to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, wherein said plant exhibits a difference in a response to SD+EODFR light conditions as compared to a corresponding response in a control plant that does not comprise said exogenous nucleic acid.

4-8. (canceled)

9. The plant of claim 1, wherein said regulatory region is a promoter.

10. (canceled)

11. The plant of claim 1, wherein said plant is a dicot.

12. The plant of claim 11, wherein said plant is a member of the genus Brassica, Glycine, Gossypium, Helianthus, Lactuca, or Medicago.

13. The plant of claim 1, wherein said plant is a monocot.

14. The plant of claim 13, wherein said plant is a member of the genus Avena, Cocos, Elaeis, Hordeum, Oryza, Panicum, Secale, Sorghum, Triticum, or Zea.

15. The plant of claim 1, wherein said difference in response to SD+EODFR conditions is a difference in hypocotyl length.

16. The plant of claim 1, wherein said difference in response to SD+EODFR conditions is a difference in petiole length.

17. Progeny of the plant of claim 1, wherein said progeny exhibit said difference relative to a said control plant under SD+EODFR conditions.

18. (canceled)

19. Vegetative tissue from a plant according to claim 1.

20-22. (canceled)

23. A method of producing a crop, said method comprising: growing a plurality of plants comprising an exogenous nucleic acid comprising a regulatory region operably linked to a nucleotide sequence encoding a polypeptide having 80 percent or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, wherein said plurality of plants exhibits a difference in a response to SD+EODFR light conditions as compared to a corresponding response in a control plant that does not comprise said exogenous nucleic acid; and harvesting said crop from said plurality of plants.

24-25. (canceled)

26. A method of producing a plant, said method comprising growing a plant cell comprising an exogenous nucleic acid comprising a regulatory region operably linked to a nucleotide sequence encoding a polypeptide having 80% or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, wherein a plant produced from said cell exhibits a difference in a response to SD+EODFR light conditions as compared to a corresponding response in a control plant that does not comprise said exogenous nucleic acid.

27-28. (canceled)

29. A method of modulating the SD+EODFR tolerance of a plant, said method comprising introducing into a plant cell an exogenous nucleic acid comprising a regulatory region operably lined to a nucleotide sequence encoding a polypeptide having 80% or greater sequence identity to an amino acid sequence selected from the group consisting of SEQ ID NO:79, SEQ ID NO:80, SEQ ID NO:81, SEQ ID NO:82, SEQ ID NO:83, SEQ ID NO:84, SEQ ID NO:87, SEQ ID NO:89, SEQ ID NO:90, SEQ ID NO:91, SEQ ID NO:92, SEQ ID NO:94, SEQ ID NO:104, SEQ ID NO:106, SEQ ID NO:109, SEQ ID NO:111, SEQ ID NO:113, SEQ ID NO:114, SEQ ID NO:116, SEQ ID NO:117, SEQ ID NO:120, SEQ ID NO:121, SEQ ID NO:122, SEQ ID NO:124, SEQ ID NO:126, SEQ ID NO:127, SEQ ID NO:128, SEQ ID NO:129, SEQ ID NO:130, SEQ ID NO:133, SEQ ID NO:135, SEQ ID NO:137, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:149, SEQ ID NO:151, SEQ ID NO:153, SEQ ID NO:154, SEQ ID NO:156, SEQ ID NO:158, SEQ ID NO:160, SEQ ID NO:162, SEQ ID NO:164, SEQ ID NO:165, SEQ ID NO:167, SEQ ID NO:168, SEQ ID NO:169, SEQ ID NO:170, and SEQ ID NO:171, wherein a plant produced from said plant cell exhibits a difference in a response to SD+EODFR light conditions as compared to a corresponding response in a control plant that does not comprise said exogenous nucleic acid.

30. (canceled)

31. A method of modulating the SD+EODFR tolerance of a plant, said method comprising introducing into a plant cell an exogenous nucleic acid, said exogenous nucleic acid comprising a regulatory region operably linked to a nucleotide sequence having 80 percent or greater sequence identity to a nucleotide sequence selected from the group consisting of SEQ ID NO:78, SEQ ID NO:85, SEQ ID NO:86, SEQ ID NO:88, SEQ ID NO:93, SEQ ID NO:102, SEQ ID NO:103, SEQ ID NO:105, SEQ ID NO:107, SEQ ID NO:108, SEQ ID NO:110, SEQ ID NO:112, SEQ ID NO:115, SEQ ID NO:119, SEQ ID NO:123, SEQ ID NO:125, SEQ ID NO:131, SEQ ID NO:132, SEQ ID NO:134, SEQ ID NO:136, SEQ ID NO:138, SEQ ID NO:148, SEQ ID NO:150, SEQ ID NO:152, SEQ ID NO:155, SEQ ID NO:157, SEQ ID NO:159, SEQ ID NO:161, SEQ ID NO:163, and SEQ ID NO:166, or a fragment thereof, wherein a plant produced from said cell exhibits a difference in a response to SD+EODFR light conditions as compared to a corresponding response in a control plant that does not comprise said exogenous nucleic acid.

32-36. (canceled)

37. The method of claim 26, wherein said regulatory region is a promoter.

38. (canceled)

39. The method of claim 26, wherein said plant is a dicotyledonous plant.

40. The method of claim 39, wherein said plant is a member of the genus Brassica, Glycine, Gossypium, Helianthus, Lactuca, or Medicago.

41. The method of claim 26, wherein said plant is a monocotyledonous plant.

42. The method of claim 41, wherein said plant is a member of the genus Avena, Cocos, Elaeis, Hordeum, Oryza, Panicum, Secale, Sorghum, Triticum, or Zea.

43. The method of claim 26, wherein said difference in response to SD+EODFR conditions is a difference in hypocotyl length.

44. The method of claim 26, wherein said difference in response to SD+EODFR conditions is a difference in petiole length.

45-46. (canceled)

47. The plant of claim 15 or 16, wherein said difference is a decrease.
